(19) United States
(12) Patent Application Publication
(10) Pub. No.: US 2026/0357343 A1
(43) Pub. Date: Nov. 12, 2026

(54) BACTERIAL SPECIES DIAGNOSTIC OF CANINE PERIODONTITIS VIA QUANTITATIVE POLYMERASE CHAIN REACTION

(71) Applicant: MARS, INCORPORATED, McLean, VA (US)

(72) Inventors: Lucy Jane HOLCOMBE, Mowbray, Leicestershire (GB); Corryn Victoria WALLIS, Mowbray, Leicestershire (GB); Avika Kishorlal RUPARELL, Mowbray, Leicestershire (GB); Steve HARRIS, Mowbray, Leicestershire (GB); Alison OAKDEN, Mowbray, Leicestershire (GB); Mattew Ronald GIBBS, Mowbray, Leicestershire (GB)

(73) Assignee: MARS, INCORPORATED, McLean, VA (US)

(21) Appl. No.: 19/108,124

(22) PCT Filed: Aug. 31, 2023
(86) PCT No.: PCT/US2023/031742

§ 371 Date: Feb. 28, 2025

Related U.S. Application Data
(60) Provisional application No. 63/403,542, filed on Sep. 2, 2022.

(51) Int. Cl. C12Q 1/6883; C12Q 1/689; G16B 40/00

(52) U.S. Cl. CPC C12Q 1/6883; C12Q 1/689; G16B 40/00

(57) ABSTRACT

Methods for determining the oral health status of an animal through the compositional analysis of the oral microbiome are disclosed herein. The abundance or relative abundance of one or more microbial taxa is assayed using qPCR and compared to modelling data produced from canines having healthy or periodontal disease states.

### CROSS-REFERENCE TO RELATED APPLICATIONS

This application claims priority to U.S. Provisional Application No. 63/403,542, filed Sep. 2, 2022, the contents of which are incorporated herein by reference in its entirety.

### SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Aug. 29, 2023, is entitled “069269_0645_SL.xml”, and is 97,652 bytes in size.

### FIELD

The presently disclosed subject matter relates to the compositional analysis of the canine oral microbiome. The presently disclosed subject matter further relates to the compositional analysis of the canine oral microbiome as a diagnostic for canine periodontal disease.

### BACKGROUND

Gum disease, such as periodontal disease, is commonly seen in veterinary treatment, with prevalence of such disease estimated between 9% and 20% [1, 2]. Higher prevalence, between 44% and 100%, has been reported in studies of anesthetized dogs and through examination of post-mortem samples [3-6]. These data imply that the disease is under-diagnosed in veterinary practice, where the majority of examinations are performed on conscious dogs.

Bacterial species in dental plaque that are associated with periodontal health and disease are known in the art [7-12]. For example, International Publication No. WO 2008/137506, incorporated herein by reference in its entirety, describes presence or absence of at least one microorganism, from a sample from the mouth of a dog, wherein the microorganism which is associated with periodontal disease in a dog is one or more selected from: Peptostreptococcus sp., Synergistes sp., Clostridiales sp., Eubacterium nodatum, Selenomonas sp., Bacteroidetes sp., Odoribacter denticanis, Desulfomicrobium ovale, Moraxella sp., Bacteroides denticanoris, Fillifactor villosus, Porphyromonas canoris, Porphyromonas gulae, Treponema denticola, and Porphyromonas salivosa. International Publication No. WO 2014/199115, incorporated herein by reference in its entirety, further describes bacteria associated with disease, particularly periodontal disease, and with good oral health.

A number of techniques have been developed to assess oral disease and disorders, such as periodontal disease. Such techniques include, but are not limited to, probing of the gums and acquiring intra-oral dental radiographs. However, such methods and techniques require animals to be given a general anesthetic, require highly trained individuals, and are costly. Additionally, such methods can be stressful for both the subject animal and the owner of the subject animal. The development of a method for the diagnosis of an oral disease or disorder, such as periodontal disease, in conscious animals would therefore be appealing to both veterinarians and owners and would improve animal welfare.

### SUMMARY OF THE INVENTION

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

In one aspect, the present disclosure provides a method for determining oral health status of an animal. In a particular embodiment, the method for determining oral health status of an animal comprises quantifying one or more microbial taxa (e.g., microorganism species) from a sample to determine abundance or relative abundance of the one or more microbial taxa, comparing the abundance or relative abundance of said one or more microbial taxa in the sample to abundance or relative abundance of the microbial taxa in a control data set, and determining the oral health status of the animal. In certain embodiments, the one or more microbial taxa is quantified using quantitative polymerase chain reaction (qPCR) or DNA sequencing methods. In certain embodiments, the one or more microbial taxa is associated with periodontal health or periodontal disease. In certain embodiments, the one or more microbial taxa in the control data set is associated with periodontal health or periodontal disease.

In certain embodiments, the one or more microbial taxa are bacterial species. In certain embodiments, the one or more microbial taxa is selected from the group consisting of Actinomyces sp., Anaerovorax sp., Bacteroides sp., Bergeyella sp., Capnocytophaga sp., Clostridiales sp., Desulfomicrobium sp., Filifactor sp., Frigovirgula sp, Fusobacterium sp, Gemella sp., Granulicatella sp., Helcococcus sp., Lachnospiraceae sp., Leptotrichia sp., Moraxella sp., Neisseria sp., Odoribacter sp., Pasteurellaceae sp., Peptococcus sp., Peptostreptococcaceae sp., Peptostreptococcus sp. Porphyromonas sp., Synergistales sp., Tannerella sp., and combinations thereof. In an embodiment, the one or more microbial taxa is selected from the group consisting of Actinomyces sp. COT-083, Actinomyces sp. COT-252, Anaerovorax sp. COT-125, Bacteroides sp. COT-040, Bergeyella zoohelcum COT-186, Capnocytophaga sp. COT-339, Clostridiales sp. COT-005, Clostridiales sp. COT-028, Desulfomicrobium orale COT-008, Filifactor sp. COT-064, Frigovirgula sp. COT-007, Fusobacterium sp. COT-169, Fusobacterium sp. COT-189, Gemella palaticanis COT-089, Granulicatella sp. COT-095, Helcococcus sp. COT-069, Lachnospiraceae XIVa [G-4] sp. COT-099, Lachnospiraceae XIVa [G-5] sp. COT-024, Leptotrichia sp. COT-345, Moraxella sp. COT-017, Moraxella sp. COT-018, Neisseria animaloris COT-016, Odoribacter denticanis COT-084, Pasteurellaceae sp. COT-271, Peptococcus sp. COT-044, Peptostreptococcaceae XI [G-1] sp. COT-004, Peptostreptococcaceae XI [G-1] sp. COT-006, Peptostreptococcaceae XI [G-3] sp. COT-104, Peptostreptococcaceae XI [G-4] sp. COT-019, Peptostreptococcaceae XI [G-6] sp. COT-068, Peptostreptococcaceae XIII [G-1] sp. COT-030, Peptostreptococcaceae XIII [G-2] sp. COT-077, Peptostreptococcus sp. COT-033, Peptostreptococcus sp. COT-227, Porphyromonas cangingivalis COT-109, Porphyromonas gingivicanis COT-022, Porphyromonas gulae I COT-052, Porphyromonas macacae COT-192, Porphyromonas sp. COT-108, Synergistales [F-2,G-1] sp. COT-138, or Tannerella forsythus COT-023, and combinations thereof. In an embodiment, the one or more microbial taxa is selected from the group consisting of Clostridiales sp, COT-028, Peptostreptococcaceae XI [G-4] sp. COT-019, or Capnocytophaga sp. COT-339, and combinations thereof. In an embodiment, the abundance or relative abundance of the one or more microbial taxa is determined by amplifying and/or sequencing 16S IRNA, 16S rDNA, ITS, 18S rRNA, or 18S rDNA. In certain embodiments, the bacterial taxa are determined by amplifying and/or sequencing one or more of the V1-V3, V3-V4, or V4 regions of the 16S rDNA, such as by using 454-pyrosequencing or Illumina sequencing.

In certain embodiments, the one or more microbial taxa is a fungal species, an archaea species, or a protozoan species.

In certain embodiments, the animal is a domestic animal. In certain embodiments, the domestic animal is a dog, cat, horse, cow, ferret, rabbit, pig, rat, mouse, gerbil, hamster, or goat. In certain embodiments, the animal is a wild animal. In certain embodiments, the wild animal is a wolf, bison, elk, deer, lion, or tiger.

In certain embodiments, the presently disclosed methods further comprise extracting DNA from the sample. In certain embodiments, the sample is a gingival area sample, e.g., gingival margin, a subgingival area sample, a supragingival area sample, a saliva sample, a tongue sample, or a buccal sample. In certain embodiments, wherein the sample is obtained from a conscious animal or from an unconscious animal. In certain embodiments, the animal has or is suspected to have gingivitis and/or periodontitis. In certain embodiments, the oral health status comprises periodontal disease.

In another aspect, the present disclosure provides methods implemented by a computer system for determining the animal oral health state. In certain embodiments, the methods comprise receiving input data for an animal, wherein the input data comprises at least a first array comprising a first plurality of entries, and each entry comprises a numerical value that indicates an amount or relative abundance of a type of one or more microbial taxa; determining, by a machine learning model, an animal oral health state based on the input data for the animal, wherein the animal oral health state identifies a predicted oral health state classification for the animal; obtaining the animal oral health state from the machine learning model; and outputting the animal oral health state.

In certain embodiments, the input data comprises quantified one or more microbial taxa associated with periodontal health or periodontal disease from the sample. In certain embodiments, the one or more microbial taxa is quantified using qPCR or DNA sequencing methods. In certain embodiments, the bacterial taxa are determined by amplifying and/or sequencing one or more of the V1-V3, V3-V4, or V4 regions of the 16S rDNA, such as by using 454-pyrosequencing or Illumina sequencing. In certain embodiments, the one or more microbial taxa in the control data set is associated with periodontal health or periodontal disease.

In certain embodiments, the methods further comprise: obtaining training data for a plurality of animals, wherein the training data indicates an amount or relative abundance of a type of microorganism that is present within a sample for each animal from among the plurality of animals; associating the training data with animal oral health state classifications, wherein associating the training data with the animal oral health state classifications comprises associating each animal from among the second plurality of animals with an animal oral health state classification; and training the machine learning model using the training data that is associated with the animal oral health state classifications.

In certain embodiments, the methods further comprise: identifying, by the machine learning model, particular microbial taxa in canine plaque that are significantly associated with health, gingivitis, and/or periodontitis for determining the oral health state of the animal.

In certain embodiments, the input data further comprises one or more of an animal breed identifier, an animal size, an animal weight, an animal age, animal health information, animal diet, a geographical location information, a sample location, or a combination thereof. In certain embodiments, the input data comprise abundance or relative abundance of the one or more microbial taxa. In certain embodiments, the one or more microbial taxa are bacterial species.

In certain embodiments, the one or more microbial taxa is selected from the group consisting of Actinomyces sp., Anaerovorax sp., Bacteroides sp., Bergeyella sp., Capnocytophaga sp., Clostridiales sp., Desulfomicrobium sp., Filifactor sp., Frigovirgula sp, Fusobacterium sp, Gemella sp., Granulicatella sp., Helcococcus sp., Lachnospiraceae sp., Leptotrichia sp., Moraxella sp., Neisseria sp., Odoribacter sp., Pasteurellaceae sp., Peptococcus sp., Peptostreptococcaceae sp., Peptostreptococcus sp. Porphyromonas sp., Synergistales sp., Tannerella sp., and combinations thereof. In certain embodiments, the one or more microbial taxa is selected from the group consisting of Actinomyces sp. COT-083, Actinomyces sp. COT-252, Anaerovorax sp. COT-125, Bacteroides sp. COT-040, Bergeyella zoohelcum COT-186, Capnocytophaga sp. COT-339, Clostridiales sp. COT-005, Clostridiales sp. COT-028, Desulfomicrobium orale COT-008, Filifactor sp. COT-064, Frigovirgula sp. COT-007, Fusobacterium sp. COT-169, Fusobacterium sp. COT-189, Gemella palaticanis COT-089, Granulicatella sp. COT-095, Helcococcus sp. COT-069, Lachnospiraceae XIVa [G-4] sp. COT-099, Lachnospiraceae XIVa [G-5] sp. COT-024, Leptotrichia sp. COT-345, Moraxella sp. COT-017, Moraxella sp. COT-018, Neisseria animaloris COT-016, Odoribacter denticanis COT-084, Pasteurellaceae sp. COT-271, Peptococcus sp. COT-044, Peptostreptococcaceae XI [G-1] sp. COT-004, Peptostreptococcaceae XI [G-1] sp. COT-006, Peptostreptococcaceae XI [G-3] sp. COT-104, Peptostreptococcaceae XI [G-4] sp. COT-019, Peptostreptococcaceae XI [G-6] sp. COT-068, Peptostreptococcaceae XIII [G-1] sp. COT-030, Peptostreptococcaceae XIII [G-2] sp. COT-077, Peptostreptococcus sp. COT-033, Peptostreptococcus sp. COT-227, Porphyromonas cangingivalis COT-109, Porphyromonas gingivicanis COT-022, Porphyromonas gulae I COT-052, Porphyromonas macacae COT-192, Porphyromonas sp. COT-108, Synergistales [F-2,G-1] sp. COT-138, Tannerella forsythus COT-023, and combinations thereof. In certain embodiments, the one or more microbial taxa is selected from the group consisting of Clostridiales sp, COT-028, Peptostreptococcaceae XI [G-4] sp. COT-019, Capnocytophaga sp. COT-339, and combinations thereof.

In certain embodiments, the one or more microbial taxa is a fungal species, an archaea species, or a protozoan species.

In certain embodiments, the abundance or relative abundance of the one or more microbial taxa is determined by amplifying 16S rRNA, 16S rDNA, ITS, 18S rRNA, or 18S rDNA

In certain embodiments, the animal is a domestic animal. In certain embodiments, the domestic animal is a dog, cat, horse, cow, ferret, rabbit, pig, rat, mouse, gerbil, hamster, or goat. In certain embodiments, the animal is a wild animal. In certain embodiments, the wild animal is a wolf, bison, elk, deer, lion, or tiger.

In certain embodiments, the one or more microbial taxa is associated with periodontal health or periodontal disease. In certain embodiments, the one or more microbial taxa in the control data set is associated with periodontal health or periodontal disease. In certain embodiments, the one or more microbial taxa is present in a sample. In certain embodiments, the sample is a gingival area sample, a gingival margin sample, a subgingival area sample, a supragingival area sample, a saliva sample, a tongue sample, or a buccal sample. In certain embodiments, the sample is obtained from a conscious animal or from an unconscious animal. In certain embodiments, the animal has or is suspected to have gingivitis and/or periodontitis.

In another aspect, the present disclosure provides a system comprising: one or more non-transitory computer-readable storage media including instructions; and one or more processors coupled to the storage media, the one or more processors configured to execute the instructions to: receive input data for an animal, wherein the input data comprises at least a first array comprising a first plurality of entries, and each entry comprises a numerical value that indicates an amount of a type of one or more microbial taxa; determine, by a machine learning model, an animal oral health state based on the input data for the animal, wherein the animal oral health state identifies a predicted oral health state classification for the animal; obtain the animal oral health state from the machine learning model; and output the animal oral health state.

In another aspect, the present disclosure provides a system comprising one or more non-transitory computer-readable storage media including instructions; and one or more processors coupled to the storage media, the one or more processors configured to execute instructions to perform any of the methods disclosed herein.

In a further aspect, the present disclosure provides a non-transitory computer-readable medium comprising: instructions that, when executed by one or more processors of a computing system, cause the one or more processors to: receive input data for an animal, wherein the input data comprises at least a first array comprising a first plurality of entries, and each entry comprises a numerical value that indicates an amount of a type of one or more microbial taxa; determine, by a machine learning model, an animal oral health state based on the input data for the animal, wherein the animal oral health state identifies a predicted oral health state classification for the animal; obtain the animal oral health state from the machine learning model; and output the animal oral health state.

In a further aspect, the present disclosure provides a non-transitory computer-readable medium comprising instructions that, when executed by one or more processors of a computing system, cause the one or more processors to perform any of the methods disclosed herein.

Certain embodiments of the present disclosure can include some, all, or none of these advantages. These advantages and other features will be more clearly understood from the following detailed description taken in conjunction with the accompanying drawings and claims.

### BRIEF DESCRIPTION OF THE DRAWINGS

The following figures are included to illustrate certain aspects of the present disclosure and shown not be viewed as exclusive embodiments. The subject matter disclosed is capable of considerable modifications, alterations, combinations, and equivalents in form and function, without departing from the scope of this disclosure.

FIG. 1 provides principal component scores with ellipses representing 95% confidence regions from analysis performed on the counts and proportions identified via high-throughput sequencing (HTS) and quantitative polymerase chain reaction (qPCR) respectively. Discriminated by health state: health (green), gingivitis (orange) and periodontitis (red); and analytical method: qPCR (empty data points; dotted ellipse) and HTS (filled data points; solid ellipse).

FIGS. 2A-2C provide correlations between high-throughput sequencing (HTS) and quantitative polymerase chain reaction (qPCR) datasets acquired from analysis of the same sample cohort for the following bacteria: 2A) Capnocytophaga sp. COT-339, 2B) Peptostreptococcaceae XI [G-4] sp. COT-019 and 2C) Clostridiales sp. COT-028. Samples are discriminated by periodontal health state: health (green), gingivitis (orange) and periodontitis (red).

FIG. 3 provides Sensitivity against 1—Specificity estimations using 5 machine learning classification models, for Peptostreptococcaceae XI [G-4] sp. COT-019, Clostridiales sp. COT-028 and Capnocytophaga sp. COT-339. Methods employed were logistic regression (LR, red), weighted k-nearest neighbour (KKNN, dark blue), kernel support vector machines (KSVM, yellow), linear discriminant analysis (LDA, purple) and random forest (RF, aqua). Average estimates+/−standard deviation are presented from the bootstrap testing of the optimised trained models.

FIG. 4 is a schematic diagram of an embodiment of a diagnostic system 100 that is configured to perform animal diagnostics using machine learning.

FIG. 5 is a flowchart of an embodiment of an oral health state determination process 200 for the diagnostic system 100.

FIG. 6 is an embodiment of a network device 102 for the diagnostic system 100.

### DETAILED DESCRIPTION

The presently disclosed subject matter relates to methods for sampling the oral microbiome and monitoring oral health in animals. The presently disclosed subject matter is particularly suited for sampling the oral microbiome of a companion animal, e.g., a domestic dog.

### 1. DEFINITIONS

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

References to a percentage sequence identity between two nucleotide sequences mean that, when aligned, that percentage of nucleotides are the same in comparing the two sequences. This alignment and the percent homology or sequence identity can be determined using any suitable software programs. For example, those described in section 7.7.18 of reference [13]. In one embodiment, an alignment is determined using the BLAST algorithm or the Smith-Waterman homology search algorithm using an affine gap search with a gap open penalty of 12 and a gap extension penalty of 2, BLOSUM matrix of 62. The Smith-Waterman homology search algorithm is disclosed in reference [14]. The alignment can be over the entire reference sequence, i.e., it can be over 100% length of the sequences disclosed herein.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms. Further, the term “comprising” encompasses “including” as well as “consisting,” e.g., a composition “comprising” X can consist exclusively of X or can include something additional, e.g., X+Y.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “taxa” refers to taxonomical groups, for example, kingdom, phylum, class, order, family, genus, and species. The term “abundance” can refer to an absolute amount (including presence or absence) of given bacterial taxa present within a sample. For example, an abundance can refer to the count of bacterial sequences of bacterial taxa after appropriate amplification of 16S rDNA. The term “relative abundance” can refer to a percentage composition of particular bacterial taxa (e.g., species) relative to the total number of bacteria in the sample. It can be calculated by determining the number of sequences of given bacterial taxa divided by the total number of all bacterial sequences which is then multiplied by 100. For example, the relative abundance can refer to the relative amounts of nucleic acid present in a sample after appropriate amplification or sequencing of 16S rDNA. In certain embodiments, the relative abundance can refer to a binary classification of bacteria taxa. For example, without any limitation, binary classification can include detected versus undetected taxa or presence versus absence of taxa. In certain embodiments, the relative abundance is calculated as odds ratio. As used herein, odds ratio can be a fold change, i.e., it is a measure of how much higher or lower the abundance or relative abundance is when comparing one group to another group. In certain embodiments, the 16S rRNA comprises or consists of one of the sequences provided in Table 1.

TABLE 1
16S rRNA sequences.

| | Truncated | |
|---|---|---|
| | Assay | |
| Full Assay Name | Name | 16S rRNA sequence |
| Actinomyces sp. | COT-083 | >083OC035 \| Actinomyces sp. \| COT-083 \| Clone OC035 \| JN713246 \| 1 \| |
| COT-083 | | GACGAACGCTGGCGGCGTGCTTAACACATGCAAGTCGAACGGGATCTGATGG |
| (SEQ ID NO.: 1) | | GGCTTTTGCTTTGTTGGTGAGAGTGGCGAACGGGTGAGTAACACGTGAGTAA |
| | | CCTGCCCTCAACATTTGAATAACTCCATGAAAGTGGGGCTAATGCAATATATT |
| | | CTGCAGTCATCGCATGGTGGTTGTGGGAAACGGTTTTTTCTGGTTGGGGATGG |
| | | \|GCTCGCGGCCTATCAGCTTGTTGGTGGGGTGATGGCCTACCAAGGCGGTGAC |
| | | GGGTAGCCGGCCTGAGAGGGTGGACGGCCACACTGGGACTGAGATACGGCC |
| | | CAGACTCCTACGGGAGGCAGCAGTGGGGGATTTTGCACAATGGGCGCAAGC |
| | | CTGATGCAGCGACGCCGCGTGAGGGATGACGGCCTTCGGGTTGTAAACCTCT |
| | | TTCGCTAGGTTGGAAGACGCTTTTTGGAGTTGTTGACTTTAACTGGTAAAGAA |
| | | GTACCGGCTAACTACGTGCCAGCAGCCGCGGTAATACGTAGGGTACTAGCGT |
| | | TGTCCGGAATTATTGGGCGTAAAGGGCTTGTAGGTGGTTTGTCGCGTCTGTCG |
| | | TGAAAACCCGGGGCTTAACTCCGGGCTTGCGGTGGGTACAGGCAGGCTTGAG |
| | | TGCGGTAGGGGAGACTGGAATTCCTGGTGTAGCGGTGGAATGCGCAGATATC |
| | | AGGAGGAATACCGGTGGCGAAGGCGGGTCTCTGGGCCGTTACTGACACTGA |
| | | GGAGCGAAAGCGTGGGGAGCGAACAGGATTAGATACCCTGGTAGTCCACGC |
| | | TGTAAACGTTGGGCACTAGTTGTGGGGGCTTTTGTTGGTTTCCGCGACGTAGC |
| | | TAACGCATTAAGTGCCCCGCCTGGGGAGTACGGCCGCAAGGCTAAAACTCAA |
| | | AGGAATTGACGGGGACCCGCACAAGCGGCGGAGCATGCGGATTAATTCGAT |
| | | GCAACGCGAAGAACCTTACCAAGGCTTGACATGCACCGGACGGCTCTAGAGA |
| | | TAGGGTTTCCCTTTGTGGCTGGTGTGCAGGTGGTGCATGGTTGTCGTCAGCTC |
| | | GTGTCGTGAGATGTTGGGTTAAGTCCCGCAACGAGCGCAACCCTTGTCTAGTG |
| | | TTGCCAGCAAGTAGTGTTGGGGACTCTCTAGTGACTGCCGGGGTTAACTCGGA |
| | | GGAAGGTGGGGATGACGTCAAATCATCATGCCCCTTATGTCTTGGGCTTCACG |
| | | CATGCTACAATGGGCGGTACAGTGGGTTGCAATACCGTGAGGTGGAGCTAAT |
| | | CCCCAAAGCCGTTCTCAGTTCGGATCGTAGTCTGCAACTCGACTACGTGAAGG |
| | | TGGAGTCGCTAGTAATCGCAGATCAGCATTGCTGCGGTGAATACGTTCTCGGG |
| | | TCTTGTACACACCGCCCGTCACGTCACGAAAGTTGGTAACACCCGAAGCTCAT |
| | | GGCCTAACCGCGTTTGTGGGGGGAGTGGTCGAAGGTGGGATTGGCGATTGGG |
| | | ACGAAGTCGTAGCAAGGTAGCTGTACCGGAAGGTGCGGCTGGAACACCTCCT |
| | | T |
| Actinomyces sp. | COT-252 | >252ZI340 \| Actinomyces sp. \| COT-252 \| Clone ZI340 \| JN713416 \| 2 \| |
| COT-252 | | GACGAACGCTGGCGGCGTGCTTAACACATGCAAGTCGAACGGGATCCAAGTG |
| (SEQ ID NO.: 2) | | GGCTTGCTTGCTTGGTGAGAGTGGCGAACGGGTGAGTAACACGTGAGTAACC |
| | | TGCCCCCTTCTTTGGGATAACGACCAGAAATGGTTGCTAATACTGAATATTCAC |
| | | GCATTGTCGCATGGTGGTGTGTGGAAAGGTTTTTCTGGTGGGGGATGGGCTCG |
| | | CGGCCTATCAGCTTGTTGGTGGGGTGATGGCCTACCAAGGCTTTGACGGGTA |
| | | GCCGGCCTGAGAGGGTGACCGGTCACACTGGGACTGAGATACGGCCCAGAC |
| | | TCCTACGGGAGGCAGCAGTGGGGAATATTGCACAATGGGCGAAAGCCTGATG |
| | | CAGCGACGCCGCGTGAGGGATGACGGCCTTCGGGTTGTAAACCTCTTTCAGT |
| | | AAGGACGCAGGCCTAGGCTTTTGTTTGGGTTGACTTTACTTACAGAAGAAGCG |
| | | CCGGCTAACTACGTGCCAGCAGCCGCGGTAATACGAAGGGCGCAAGCGTTGT |
| | | CCGGAATTATTGGGCGTAAAGAGCTCGTAGGCGGTTTGTCGCGTCTGTCGTGA |
| | | AAACCCACAGCTTAACTGTGGGCTTGCGGTGGGTACGGGCAGGCTTGAGTGC |
| | | GGTAGGGGAGACTGGAATTCCTGGTGTAGCGGTGGAATGCGCAGATATCAGG |
| | | AGGAACACCGGTGGCGAAGGCGGGTCTCTGGGCCGTTACTGACGCTGAGGA |
| | | GCGAAAGCATGGGGAGCGAACAGGATTAGATACCCTGGTAGTCCATGCCGTA |
| | | AACGTTGGGCACTAGGTGTGGGGGCTGTTTCCATGGTTCCTGCGCCGTAGCTA |
| | | ACGCATTAAGTGCCCCGCCTGGGGAGTACGGTCCCAAGGCTAAAACTCAAAG |
| | | GAATTGACGGGGGCCCGCACAAGCGGCGGAGCATGCGGATTAATTCGATGC |
| | | AACGCGAAGAACCTTACCAAGGCTTGACATACATCCAGAGTCTGCAGAGATG |
| | | TGGGCGCATTTAGTTGGGGGTGTACAGGTGGTGCATGGTTGTCGTCAGCTCGT |
| | | GTCGTGAGATGTTGGGTTAAGTCCCGCAACGAGCGCAACCCTTGTCTCATGTT |
| | | GCCAGCACGTTATGGTGGGGACTCGTGAGAGACTGCCGGGGTTAACTCGGAG |
| | | GAAGGTGGGGATGACGTCAAATCATCATGCCCCTTATGTCTTGGGCTTCACGC |
| | | ATGCTACAATGGCCAGTACAGAGGGTTGCGATATCGTAAGGTTGAGCGAATC |
| | | CCTTAAAGCTGGTCTCAGTTCGGATTGGGGTCTGCAACTCGACCCCATGAAGT |
| | | CGGAGTCGCTAGTAATCGCAGATCAGCAACGCTGCGGTGAATACGTTCTCGG |
| | | GCCTTGTACACACCGCCCGTCACGTCACGAAAGTTGGTAACACCCGAAGCCC |
| | | ATGGCCTAACCGTTTGGGGGGAGTGGTCGAAGGTGGGACTGGCGATTGGGAC |
| | | GAAGTCGTAACAAGGT |
| Anaerovorax sp. | COT-125 | > 125OJ076 \| Peptostreptococcaceae XI [G-10] sp. \| COT-125 \| Clone |
| COT-125 | | OJ076 \| JN713292 \| 28 \| |
| (SEQ ID NO.: 3) | | GAGGAACGCTGGCGGCGTGCCTAACACATGCAAGTCGAGCGAGAAATAGTC |
| | | AGACGGAGATTTCGGTTGAAGGATGGTTATGGAAAGCGGCGGACGGGTGAG |
| | | TAACGCGTAGGCAACCTGCCTCATGCAGAGGGATAGCCTCGGGAAACCGGG |
| | | ATTAAAACCTCATAAGGCCAAGGGGACACATGTTTTCGTGGTCAAAGATTTAT |
| | | CGGCATGAGATGGGCCTGCGTCTGATTAGCTGGTTGGTGAGGTAACGGCTCA |
| | | CCAAGGCGACGATCAGTAGCCGACCTGAGAGGGTAAACGGCCACATTGGAA |
| | | CTGAGACACGGTCCAAACTCCTACGGGAGGCAGCAGTGGGGAATATTGCACA |
| | | ATGGGCGAAAGCCTGATGCAGCAACGCCGCGTGAGCGAAGAAGGCCTTTGG |
| | | GTCGTAAAGCTCTGTCGATAGGGAAGAAAAAGGTGACGGTACCTATCAAGAA |
| | | AGCCCCGGCTAACTACGTGCCAGCAGCCGCGGTAATACGTAGGGGGCGAGC |
| | | GTTATCCGGAATTATTGGGCGTAAAGAGTGCGTAGGTGGCTAGATAAGCGTG |
| | | GGGTGAAAGGCAGTGGCTTAACCATTGTAAGCCCTGCGAACTGTGTAGCTTG |
| | | AGTGCAGGAGGGGAAAGTGGAATTCCTAGTGTAGCGGTGAAATGCGTAGATA |
| | | TTAGGAGGAACACCGGTGGCGAAGGCGGCTTTCTGGACTGTAACTGACACTG |
| | | AGGCACGAAAGCGTGGGTAGCAAACAGGATTAGATACCCTGGTAGTCCACGC |
| | | CGTAAACGATGAGCACTAGGTGTTGGTCTCGCAAGAGATCGGTGCCGCAGTT |
| | | AACGCAGTAAGTGCTCCGCCTGGGGAGTACGCACGCAAGTGTAAAACTCAAA |
| | | GGAATTGACGGGGACCCGCACAAGCAGCGGAGCATGTGGTTTAATTCGAAGC |
| | | AACGCGAAGAACCTTACCAGGACTTGACATCTGCCTGACATTCCTTTAATAGG |
| | | GGAGTTCTTTTAGACAGGCAAGACAGGTGGTGCATGGTTGTCGTCAGCTCGTG |
| | | TCGTGAGATGTTGGGTTAAGTCCCGCAACGAGCGCAACCCTTGTCAATAGTTG |
| | | CCAGCAGTAAGATGGGGACTCTATTGAGACTGCCGTGGATAACACGGAGGAA |
| | | GGTGGGGATGACGTCAAATCATCATGCCCCTTATGTTCTGGGCTACACACGTG |
| | | CTACAATGGCCGGTACAGAGAGAAGCGAGATAGAGATATGGAGCAAACCTC |
| | | AGAAAACCGGTCCCAGTTCGGATTGTAGGCTGCAACTCGCCTACATGAAGTTG |
| | | GAGTTGCTAGTAATCGCAGATCAGAATGCTGCGGTGAATGCGTTCCCGGGTCT |
| | | TGTACACACCGCCCGTCACACCATGGAAGTTGGGGGTGCCCGAAGTCGGTTA |
| | | GGAAATAGGCTGCCGAAGGCAAAACCAATGACTGGGGTGAAGTCGTAACAA |
| | | GGTAGCCGTATCGGAAGGTGCGGCTGGATCACCTCCTT |
| Bacteroides sp. | COT-040 | >040OD028 \| Bacteroides sp. \| COT-040 \| Clone OD028 \| JN713206 \| 27 \| |
| COT-040 | | GATGAACGCTAGCTACAGGCTTAACACATGCAAGTCGATGGGCATCATGGTA |
| (SEQ ID NO.: 4) | | TTAGCTTGCTAATACTGATGGCGACCGGCGCACGGGTGAGTAACACGTATCC |
| | | AACCTGGCTTTCACTCAGGGATAGCCCATCGAAAGATGGATTAATACTTGATG |
| | | GTATATACATTTCGCATGTTTTGTATATTAAAGATTTATCGGTGATAGATGGGG |
| | | ATGCGTACCATTAGTTTGTTGGCGGGGTAACGGCCCACCAAGACTTCGATGGT |
| | | TAGGGGTTCTGAGAGGAAGGTCCCCCACATTGGAACTGAGACACGGTCCAAA |
| | | CTCCTACGGGAGGCAGCAGTGAGGAATATTGGTCAATGGACGTAAGTCTGAA |
| | | CCAGCCAAGTAGCGTGAAGGATGACTGCCCTCTGGGTTGTAAACTTCTTTTAT |
| | | ATGTGAATAAATTTTCCTACGTGTAGGTTTTTGCATGTAACATATGAATAAGCA |
| | | TCGGCTAACTCCGTGCCAGCAGCCGCGGTAATACGGAGGATGCGAGCGTTAT |
| | | CCGGATTTATTGGGTTTAAAGGGAGCGTAGGTGGGTTATTAAGTCAGCGTTGT |
| | | GAAAGTTTGCGGCTCAACCGTAAAATTGCCGTTGATACTGATAGTCTTGAGTT |
| | | CAGTAGAGGTGTGCGGAATTCGTGGTGTAGCGGTGAAATGCTTAGATATCAC |
| | | GAAGAACTCCAATTGCGTAGGCAGCTCACTATACTGTTACTGACACTGATGCT |
| | | CGAAAGTGTGGGTATCAAACAGGATTAGATACCCTGGTAGTCCACACAGTAA |
| | | ACGATGAATACTCGCTGTTTGCGATATAGTGTAAGCGGCCAAGCGAAAGCGTT |
| | | AAGTATTCCACCTGGGGAGTACGCCGGCAACGGTGAAACTCAAAGGAATTGA |
| | | CGGGGGCCCGCACAAGCGGAGGAACATGTGGTTTAATTCGATGATACGCGAG |
| | | GAACCTTACCTGGGCTTAAATATTAGATGCATAACATAGAGATATGTTTTTCTT |
| | | CGGACTTCTATTAAGGTGCTGCATGGTTGTCGTCAGCTCGTGCCGTGAGGTGT |
| | | CGGCTTAAGTGCCATAACGAGCGCAACCCTAATGTTTAGTTACTAACAGTTTAT |
| | | GCTGAGGACTCTAGACAGACTGCCGTCGTAAGAAGTGAGGAAGGTTAGGATG |
| | | ACGTCAAATCAGCACGGCCCTTACGTCCAGGGCTACACACGTGTTACAATGG |
| | | GGGGTACAGAAGGTTGCTACACGGCGACGTGATGCTAATCCCAAAAACCTCT |
| | | CTCAGTTCGGATTGGAGTCTGCAACCCGACTCCATGAAGCTGGATTCGCTAGT |
| | | AATCGCGCATCAGCCACGGCGCGGTGAATACGTTCCCGGGCCTTGTACACAC |
| | | CGCCCGTCAAGCCATGAAAGCCGGGGGTACCTGAAGTACGTAACCGTAAGGA |
| | | GCGTCCTAGGGTAAATCTGGTGATTGGGGCTAAGTCGTAACAAGGTAGCCGT |
| | | ACCGGAAGGTGCGGCTGGATCACCTCCTT |
| Bergeyella | COT-186 | > 186QC056 \| Bergeyella zoohelcum \| COT-186 \| Clone QC056 \| JN713352 |
| zoohelcum COT- | | \| 97 \| |
| 186 | | GATGAACGCTAGCGGGAGGCCTAACACATGCAAGCCGAGCGGGATTTGTTGG |
| (SEQ ID NO.: 5) | | TTAGCTTGCTAACTGATAATGAGAGCGGCGTACGGGTGCGTAACACGTGTGC |
| | | AACCTGCCTTTATCTGGGGGATAGCCTTTCGAAAGGAAGATTAATACCCCATA |
| | | ATATATTGATTGGCATCGATTAATATTGAAAGCTGAGGCGGATAGAGATGGGC |
| | | ACGCGTAAGATTAGCTAGTTGGTGAGGTAACGGCTCACCAAGGCTGCGATCTT |
| | | TAGGGGGCCTGAGAGGGTGATCCCCCACACTGGTACTGAGACACGGACCAG |
| | | ACTCCTACGGGAGGCAGCAGTGAGGAATATTGGACAATGGGTGAGAGCCTGA |
| | | TCCAGCCACCCCGCGTGAAGGACTAAGGACCTATGGTTTGTAAACTTCTTTTAT |
| | | ACAGGGATAAACCTACTCTCGTGAGGGTAGCTGAAGGTACTGTATGAATAAG |
| | | CACCGGCTAACTCCGTGCCAGCAGCCGCGGTAATACGGAGGGTGCAAGCGTT |
| | | ATCCGGATTTATTGGGTTTAAAGGGTCCGTAGGCGGGTCGATAAGTCAGTGGT |
| | | GAAAGCCTGCAGCTTAACTGTAGAACTGCCGTTGATACTGTCGGTCTTGAGTG |
| | | TATTTGAGGTAGCTGGAATGAGTAGTGTAGCGGTGAAATGCATAGATATTACT |
| | | CAGAACACCAATTGCGAAGGCAGGTTACCAAGTTACAACTGACGCTGATGGA |
| | | CGAAAGCGTGGGGAGCGAACAGGATTAGATACCCTGGTAGTCCACGCTGTAA |
| | | ACGATGCTAACTCGTTTTTGGGGTATTGTACTTCAGAGACCAAGCGAAAGTGA |
| | | TAAGTTAGCCACCTGGGGAGTACGAACGCAAGTTTGAAACTCAAAGGAATTG |
| | | ACGGGGGCCCGCACAAGCGGTGGATTATGTGGTTTAATTCGATGATACGCGA |
| | | GGAACCTTACCAAGACTTAAATGGGAATTGACAGCTGTAGAAATACGGTTTTC |
| | | TTCGGACAATTTTCAAGGTGCTGCATGGTTGTCGTCAGCTCGTGCCGTGAGGT |
| | | GTTAGGTTAAGTCCTGCAACGAGCGCAACCCCTGTCACTAGTTGCCATCATTA |
| | | AGTTGGGGACTCTAGTGAGACTGCCTACGCAAGTAGAGAGGAAGGTGGGGA |
| | | TGACGTCAAATCATCACGGCCCTTACGTCTTGGGCCACACACGTAATACAATG |
| | | GCCGGTACAGAGGGCAGCTACACTGCGAAGTGATGCGAATCTCGAAAGCCG |
| | | GTCTCAGTTCGGATTGGAGTCTGCAACTCGACTCTATGAAGCTGGAATCGCTA |
| | | GTAATCGCGCATCAGCCATGGCGCGGTGAATACGTTCCCGGGCCTTGTACAC |
| | | ACCGCCCGTCAAGCCATGGAAGTCTGGGGTACCTGAAGTCGGTGACCGTCAA |
| | | AGGAGCTGCCTAGGGTAAAACAGGTAACTAGGGCTAAGTCGTAACAAGGT |
| Capnocytophaga | COT-339 | > 3391I154 \| Capnocytophaga sp. \| COT-339 \| Clone 1I154 \| JN713507 \| 3 \| |
| sp. COT-339 | | GATGAACGCTAGCGGCAGGCCTAACACATGCAAGTCGAGGGGTAGAGCTACT |
| (SEQ ID NO.: 6) | | TTAGTAGTTTGAGACCGGCGCACGGGTGCGTAACGCGTATGCAATCTACCTTA |
| | | TACAGAGGGATAGCCCAGAGAAATTTGGATTAATACCTCATAGTATATTTGAG |
| | | TGGCATCATTTAGATATTAAAGATTTATCGGTATAAGATGAGCATGCGTCCCAT |
| | | TAGTTAGTTGGTGTGGTAACGGCATACCAAGACGATGATGGGTAGGGGTCCT |
| | | GAGAGGGAGATCCCCCACACTGGTACTGAGACACGGACCAGACTCCTACGG |
| | | GAGGCAGCAGTGAGGAATATTGGTCAATGGAGGCAACTCTGAACCAGCCATG |
| | | CCGCGTGCAGGAAGAATGTCCTATGGATTGTAAACTGCTTTTGTACGGGAAGA |
| | | AAACGTGCTATGTATAGCAAACTGACGGTACCGTAAGAATAAGGATCGGCTA |
| | | ACTCCGTGCCAGCAGCCGCGGTAATACGGAGGATCCGAGCGTTATCCGGAAT |
| | | CATTGGGTTTAAAGGGTCCGTAGGCGGGATTATAAGTCAGTGGTGAAAGTTTG |
| | | CAGCTTAACTGTAAAATTGCCATTGATACTGTAGTTCTTGAATTTTTGTGAAGT |
| | | AACTAGAATATGTAGTGTAGCGGTGAAATGCATAGATATTACATGGAATACCA |
| | | ATTGCGAAGGCAGGTTACTAACAAAGGATTGACGCTGATGGACGAAAGCGTG |
| | | GGTAGCGAACAGGATTAGATACCCTGGTAGTCCACGCTGTAAACGATGGATA |
| | | CTAGCTGTTTGGTTTTCGGACTGAGTGGCTAAGCGAAAGTGATAAGTATCCCA |
| | | CCTGGGGAGTACGCACGCAAGTGTGAAACTCAAAGGAATTGACGGGGGCCC |
| | | GCACAAGCGGTGGAGCATGTGGTTTAATTCGATGATACGCGAGGAACCTTAC |
| | | CAGGGCTTAAATGTAGATTGACTTATTTGGAAACAGATATTTCTTCGGACAATT |
| | | TACAAGGTGCTGCATGGTTGTCGTCAGCTCGTGCCGTGAGGTGTCAGGTTAAG |
| | | TCCTATAACGAGCGCAACCCCTGTTGTTAGTTGCCAACGAGTGATGTCGGGAA |
| | | CTCTAGCAAGACTGCCGGTGTAAACCGTGAGGAAGGTGGGGATGACGTCAAA |
| | | TCATCACGGCCCTTACGTCCTGGGCCACACACGTGCTACAATGGCCGGTACA |
| | | GAGAGCAGCCACTTGGTGACAAGGAGCGAATCTATAAAGCCGGTCACAGTTC |
| | | GGATTGGAGTCTGCAACTCGACTCTATGAAGCTGGAATCGCTAGTAATCGGAT |
| | | ATCAGCCATGATCCGGTGAATACGTTCCCGGGCCTTGTACACACCGCCCGTCA |
| | | AGCCATGGAAGCTGGGGGTACCTGAAGTCGGTGACCGCAAGGAGCTGCCTA |
| | | GGGTAAAACTGGTAACTGGGGCTAAGTCGTAACAAGGT |
| Clostridiales sp. | COT-005 | >005OB010 \| Peptostreptococcaceae XI [G-1] sp. \| COT-005 \| Clone OB010 \| |
| COT-005 | | JN713158 \| 93 \| |
| (SEQ ID NO.: 7) | | GATGAACGCTGGCGGCGTGCTTAACACATGCAAGTCGAACGGAACGTAACCG |
| | | AGGAAATTTTTCGAAGTGGAATCAGTTACGTTTAGTGGCGGACGGGTGCGTAA |
| | | CACGTGGGCAACCTGCCCTGCTTTTCGGAATAACGCCGGGAAACCGTCGCTA |
| | | ATGCCGAATGATATGAGGAGAAGGCATCTTCTTTTCATCAAAGAATTTCGAAG |
| | | CAGGATGGGCCCGCGCCGGATTAGCTGGTTGGTGAGGTAACGGCTCACCAAG |
| | | GCGACGATCCGTAGCCGACCTGAGAGGGTGATCGGCCACACTGGAACTGAG |
| | | ACACGGTCCAGACTCCTACGGGAGGCAGCAGTGGGGAATATTGCACAATGGG |
| | | CGAAAGCCTGATGCAGCAACGCCGCGTGAGCGATGAAGGCTTTCGAGTCGTA |
| | | AAGCTCTGTCGCAGGGGAAGAAAAAGACGGTACCCTGTGAGAAAGCCCCGG |
| | | CTAACTACGTGCCAGCAGCCGCGGTAATACGTAGGGGGCAAGCGTTATCCGG |
| | | AATTACTGGGCGTAAAGGGTGCGTAGGCGGCTTTTCAAGTCAAGGCTTAAAG |
| | | GCTAACGGCTCAACCGTAGTAAGGCCTTGAAACTGGAGAGCTTGAGTATCGG |
| | | AGAGGAAAGTGGAATTCCCAGTGTAGCGGTGAAATGCGTAGATATTGGGAGG |
| | | AACACCAGTGGCGAAGGCGACTTTCTGGACGATGACTGACGCTGAGGCACGA |
| | | AAGCGTGGGGAGCAAACAGGATTAGATACCCTGGTAGTCCACGCCGTAAACG |
| | | ATGAGTACTAGGTGTCAGGTGTCAAAGCTTGGTGCCGCAGTTAACACAGTAA |
| | | GTACTCCGCCTGGGGAGTACGCTCGCAAGAGTGAAACTCAAAGGAATTGACG |
| | | GGGACCCGCACAAGTAGCGGAGCATGTGGTTTAATTCGAAGCAACGCGAAGA |
| | | ACCTTACCTAAGCTTGACATCCCGTTGACCGGGGCTTAATCGCCCCTTTCCCTT |
| | | CGGGGACAGCGGAGACAGGTGGTGCATGGTTGTCGTCAGCTCGTGTCGTGAG |
| | | ATGTTGGGTTAAGTCCCGCAACGAGCGCAACCCTTGTTTTTAGTTGCCAGCGG |
| | | TTTGGCCGGGCACTCTAAAAAGACTGCCGAGGATAACTCGGAGGAAGGTGGG |
| | | GATGACGTCAAATCATCATGCCCCTTATGCTTAGGGCTACACACGTGCTACAA |
| | | TGGCTGTTACAAAGGGCCGCGAGGCCGCAAGGCGGAGCCAATCCCATAAAA |
| | | ACAGTCTCAGTTCGGATTGCAGGCTGAAACTCGCCTGCATGAAGTCGGAGTTA |
| | | CTAGTAATCGCGAATCAGCATGTCGCGGTGAATGCGTTCCCGGGTCTTGTACA |
| | | CACCGCCCGTCACACCATGGAAGCTTGGGGCACCCAAAGTCCGTGATCCAAC |
| | | GAAAGAGGAAGCGGCCTAAGGTGAAACAAGTGACTGGGGTGAAGTCGTAAC |
| | | AAGGTAGCCGTATCGGAAGGTGCGGCTGGATCACCTCCTT |
| Clostridiales sp. | COT-028 | >028OB054 \| Clostridiales [F-1, G-2] sp. \| COT-028 \| Clone OB054 \| |
| COT-028 | | JN713190 \| 105 \| |
| (SEQ ID NO.: 8) | | GACGAACGCTGGCGGCGCGCCTAACACATGCAAGTCGAGCGGGAGTGATTG |
| | | AGGGGTAACCCGAGAGCACTTTAGCGGCGGACGGGTGAGTAACGCGTGAGC |
| | | AACCTGCCTTTTACAGGGGGATAACGTGCCGAAAGGCACACTAATACCGCAT |
| | | GAGACCACAAGCATACATATGGAAGGGGTCAAAGGAGAAATCCGGTAAGAG |
| | | ATGGGCTCGCGTCCGATTAGATAGTTGGTGAGGTAACGGCTCACCAAGTCAA |
| | | CGATCGGTAGCCGACCTGAGAGGGTGATCGGCCACATTGGAACTGAGAGAC |
| | | GGTCCAAACTCCTACGGGAGGCAGCAGTGGGGAATATTGGGCAATGGGGGA |
| | | AACCCTGACCCAGCGACGCCGCGTGAAGGAAGAAGGTCTTCGGATTGTAAAC |
| | | TTCTGTTCTTGGTGAAGAGGGAAAGACGGTAGCCAAGGAGGAAGCCCCGGCT |
| | | AACTACGTGCCAGCAGCCGCGGTAATACGTAGGGGGCGAGCGTTGTCCGGA |
| | | ATGATTGGGCGTAAAGGGCGCGTAGGCGGCTGTGTAAGTCTGGAGTGAAAGT |
| | | CCTATTTTCAAGGTAGGAATTGCTTTGGAAACTGCATAGCTTGAGTGCAGGAG |
| | | AGGTTATCGGAATTCCCGGTGTAGCGGTGAAATGCATAGAGATCGGGAGGAA |
| | | CACCAGTGGCGAAGGCGGGTAACTGGACTGCAACTGACGTTGAGGCGCGAA |
| | | AGTGTGGGGAGCAAACAGGATTAGATACCCTGGTAGTCCACACTGTAAACGA |
| | | TGAATGCTAGGTGTAGGTGGTATCGACCCCATCTGTGCCGTCGTAAACACAAT |
| | | AAGCATTCCGCCTGGGGAGTACGGTCGCAAGATTAAAACTCAAAGGAATTGA |
| | | CGGGGGCCCGCACAAGCAGCGGAGCATGTGGTTTAATTCGACGCAACGCGA |
| | | AGAACCTTACCAGGTCTTGACATCCGCTTTAGGCATGCAGAGATGCATGTGGG |
| | | ACTTCGGTCCGAAGCGAGACAGGTGGTGCATGGTTGTCGTCAGCTCGTGTCGT |
| | | GAGATGTTGGGTTAAGTCCCGCAACGAGCGCAACCCTTGCCAATAGTTACTAA |
| | | CGGATAAAGCTGAGGACTCTATTGGGACTGCCGTGGACAACACGGAGGAAG |
| | | GTGGGGACGACGTCAAATCATCATGCCCCTTATGACCTGGGCTACACACGTG |
| | | CTACAATGGTCGCAACAAAGAGGAGCGAAGCCGTAAGGTGGAGCAGATCTC |
| | | AAAAAAGCGATCCCAGTTCAGATTGTGGGCTGCAACCCGCCCACATGAAGTC |
| | | GGAGTTGCTAGTAATCGCGAATCAGCATGTCGCGGTGAATACGTTCCCGGGC |
| | | CTTGTACACACCGCCCGTCACACCATGGGAGTTGATAGCGCCCGAAGGCAGA |
| | | GAGGTAACCGCAAGGGGCCATCTGACGAAGGCGAGATCGATGACTGGGGTG |
| | | AAGTCGTAACAAGGTAGCCGTATCGGAAGGTGCGGCTGGATCACCTCCTT |
| Desulfomicrobium | COT-008 | >008OB018 \| Desulfomicrobium orale \| COT-008 \| Clone OB018 \| JN713165 |
| orale COT-008 | | \| 78 \| |
| (SEQ ID NO.: 9) | | AATGAACGCTGGCGGCGTGCCTAACACATGCAAGTCGAACGAGAAAGTTCCT |
| | | CCGGGAATGAGTAGAGTGGCGCACGGGTGAGTAACGCGTGGGTAATCTGCC |
| | | CTTGGATTTGGGATAACTTCGCGAAAGTGGAGCTAATACCGGATAGTCTGGCT |
| | | TTGTAAAAGGAGTCGGTAAAGGATGCCTCTGCATATGCATTCGTCCGAGGATG |
| | | AGCCCGCGTCTCATTAGCTAGTTGGTAGGGTAACGGCCTACCAAGGCGACGA |
| | | TGAGTAGCTGGTCTGAGAGGATGATCAGCCACACTGGGACTGAAACACGGCC |
| | | CAGACTCCTGCGGGAGGCAGCAGTGGGGAATATTGCGCAATGGGCGAAAGC |
| | | CTGTCGCAGCAACGCCGTGTGAGGGATGAAGGTTTTCGGATCGTAAACCTCTG |
| | | TCTGAAGGGAAGAAGTGATGCGGGTCCAATAGGCCCGCATTTTGACGGTACC |
| | | TTCAAAGGAAGCACCGGCTAACTCCGTGCCAGCAGCCGCGGTAATACGGAGG |
| | | GTGCAAGCGTTATTCGGAATTACTGGGCGTAAAGCGCACGTAGGCGGCCTTG |
| | | TAAGTCAGGTGTGAAATCCCACGGCTCAACCGTGGAACTGCACTTGAAACTG |
| | | CGGGGCTCGAATCCTGGAGAGGGCGGCGGAATTCCTGGTGTAGGAGTGAAAT |
| | | CCGTAGATATCAGGAGGAACACCGGTGGCGAAGGCGGCCGCCTGGACAGGT |
| | | ATTGACGCTGAGGTGCGAAAGTGTGGGGAGCAAACAGGATTAGATACCCTGG |
| | | TAGTCCACACCGTAAACGATGGATACTAGGTGTCGGGGACTTGATCCTCGGTG |
| | | CCGCAGTTAACGCGTTAAGTATCCCGCCTGGGGAGTACGGTCGCAAGGCTGA |
| | | AACTCAAAGAAATTGACAGGGGCCCGCATAAGCGGTGGAGTATGTGGTTTAA |
| | | TTCGATGCAACGCGAAGAACCTTACCTGGGCTTGACATCCTGGGAATTCCGCA |
| | | GAGATGCGGAAGTGCCCTTCGGGGAATCCAGAGACAGGTGCTGCATGGCTGT |
| | | CGTCAGCTCGTGCCGTGAGGTGTTGGGTTAAGTCCCGCAACGAGCGCAACCC |
| | | CTATCCTCAGTTGCCAGCAGGTAAAGCTGGGCACTCTGTGGAGACTGCCCGG |
| | | GTTAACCGGGAGGAAGGTGGGGACGACGTCAAGTCATCATGGCCCTTACGCC |
| | | CAGGGCTACACACGTACTACAATGGTGGGCACAAAGGGCAGCGAAACCGCA |
| | | AGGTCCAGCCAATCCCAAAAAACCCATCATAGTCCGGATTGCAGTCTGCAACT |
| | | CGACTGCAAGAAGTTGGAATCGCTAGTAATCCCGGATCAGCATGCCGGGGTG |
| | | AATACGTTCCCGGGCCTTGTACACACCGCCCGTCACACCACGAAAGTCGGTTC |
| | | TACCCGAAGCCGCCAGGCTAACCCGCAAGGGATGCAGGCGTCTACGGTAGG |
| | | GCTGGTAATTGGGGTGAAGTCGTAACAAGGTAGCCGTAGGGGAACCTGCGGC |
| | | TGGATCACCTCCTT |
| Filifactor sp. | COT-064 | >064OF011 \| Filifactor sp. \| COT-064 \| Clone OF011 \| JN713228 \| 8 \| |
| COT-064 | | GATGAACGCTGGCGGCGTGCTTAACACATGCAAGTCGAACGGACGAATCTTA |
| (SEQ ID NO.: 10) | | TCGGAGTTTTTCGGAACAAAGAAAAGATAAGTTAGTGACGGACGGGTGCGTA |
| | | ACGTGTGGGTAATCTGCCTTTGTCATGGGAATAACTGCTTGAAAGAGCAGCTA |
| | | ATACCGAATAACATATGCAATAGGCATCTATTGGATATCAAAGAGAAATCGGA |
| | | CAAAGATGAGCCCGCATCTGATTAGCTAGTTGGTAGGGTAACAGCCTACCAG |
| | | GGCGACGATCAGTAGCCGACCTGAGAGGGTGAACGGCCACATTGGAACTGA |
| | | GACACGGTCCAAACTCCTACGGGAGGCAGCAGTGGGGAATATTGCACAATGG |
| | | GGGGAACCCTGATGCAGCAACGCCGCGTGAGTGAAGAAGGCTTTCGAGTCGT |
| | | AAAACTCTGTTGTAAGGGAAGATAATGACAGTACCTTAAAAGAAAGCCCCGG |
| | | CTAACTACGTGCCAGCAGCCGCGGTAATACGTAGGGGGCAAGCGTTATCCGG |
| | | AATCACTGGGCGTAAAGGGTGCGCAGGTGGTCTGTCAAGTTAGAGGTGAAAG |
| | | GCATAGGCTCAACCAATGTAAGCCTTTAAAACTGACGGACTTGAGTGCAGGA |
| | | GAGGAAAGTGGAATTCCTAGTGTAGCGGTGAAATGCGTAGATATTAGGAGGA |
| | | ATACCGGTGGCGAAGGCGACTTTCTGGACTGTAACTGACACTGAGGCACGAA |
| | | AGCGTGGGGAGCAAACAGGATTAGATACCCTGGTAGTCCACGCCGTAAACGA |
| | | TGAGTACTAGGTGTCGGGGGCGACCTCGGTGCCGCAGTTAACACATTAAGTA |
| | | CTCCGCCTGGGGAGTACGCTCGCAAGAGTAAAACTCAAAGGAATTGACGGGG |
| | | ACCCGCACAAGCAGCGGAGCATGTGGTTTAATTCGAAGCAACGCGAAGAACC |
| | | TTACCTAAACTTGACATACCGATGACGATTCGGTAATGCGAATATTCCTTTCGG |
| | | GGACATCGGATACAGGTGGTGCATGGTTGTCGTCAGCTCGTGTCGTGAGATGT |
| | | TGGGTTAAGTCCCGCAACGAGCGCAACCCTTGTCAATAGTTGCCAGCACTTAG |
| | | GGTGGGGACTCTATTGAGACAGCTAAGGACAACTTAGAGGAAGGTGGGGAT |
| | | GACGTCAAATCATCATGCCCCTTATGTTTAGGGCTACACACGTGCTACAATGG |
| | | TCATTACAGAGGGAGGCGAAGCTGTGAAGCGGAGCGAAGCCCAAAAAGATG |
| | | ATCTAAGTTCGGATTGTAGGCTGAAACTCGCCTACATGAAGTTGGAGTTGCTA |
| | | GTAATCGCGAATCAGCATGTCGCGGTGAATGCGTTCCCGGGTCTTGTACGCAC |
| | | CGCCCGTCACACCATGGGAGTTCGGGGGGCCCAAAGTCAGTGAGCAAACCG |
| | | AGAGGGTGCAGCTGCCTAAGGCAAAACGAATGACTGGGGTGAAGTCGTAAC |
| | | AAGGTAGCCGTATCGGAAGGTGCGGCTGGATCACCTCCTT |
| Frigovirgula sp. | COT-007 | >0071F062 \| Frigovirgula sp. \| COT-007 \| Clone 1F062 \| JN713160 \| 310 \| |
| COT-007 | | GATGAACGCTGGCGGCGTGCTTAACACATGCAAGTCGAGCGGAGTTTACAAT |
| (SEQ ID NO.: 11) | | GGAAAGATCTTCGGAAATGGAAGCTGTAAACTTAGCGGCGGACGGGTGCGTA |
| | | ACGCGTGGGTAATCTGCCCTTATCATCGGGATAACACAACGAAAGATGTGCTA |
| | | ATACCGAATAACATGCATTTTCGGCATCGAAGATGTATCAAAGTGTTAGCGGA |
| | | TAAGGATGAGCCTGCGTCTGATTAGCTTGTTGGTGGGGTAATGGCCTACCAAG |
| | | GCGACGATCAGTAGCCGACCTGAGAGGGTGATCGGCCACACTGGAACTGAG |
| | | ACACGGTCCAGACTCCTGCGGGAGGCAGCAGTGGGGAATATTGCACAATGG |
| | | GCGCAAGCCTGATGCAGCAACGCCGCGTGAGCGATGAAGGTCTTCGGATCGT |
| | | AAAGCTCTGTCGCATGGGGAGATAATGACGGTACCATGTGAGGAAGCCCCGG |
| | | CTAACTACGTGCCAGCAGCCGCGGTAATACGTAGGGGGCAAGCGTTATCCGG |
| | | AATTACTGGGCGTAAAGGGTGCGTAGGTGGCCTGACAAGTCAGGGGTCAAAG |
| | | GCAACGGCTCAACCGTTGTAAGCCTTTGAAACTGTCGGGCTTGAGTTCTGGAG |
| | | GGGAAATCGGAATTCCTAGTGTAGCGGTGAAATGCGTAGATATTAGGAGGAA |
| | | CACCGGTGGCGAAGGCGGATTTCTGGACAGATACTGACACTGAGGCACGAAA |
| | | GCGTGGGGAGCAAACAGGATTAGATACCCTGGTAGTCCACGCCGTAAACGAT |
| | | GAGTACTAGGTGTTGGCCCGTTAGGGTCGGTGCCGCAGTTAACACATTAAGTA |
| | | CTCCGCCTGGGAAGTACGCTCGCAAGAGTGAAACTCAAAGGAATTGACGGGG |
| | | ACCCGCACAAGTAGCGGAGCATGTGGTTTAATTCGAAGCAACGCGAAGAACC |
| | | TTACCTAAGCTTGACATCCCTTTGACCGCAGGGTAATGCCTGCTTTCCCTTCGG |
| | | GGACAGAGGAGACAGGTGGTGCATGGTTGTCGTCAGCTCGTGTCGTGAGATG |
| | | TTGGGTTAAGTCCCGCAACGAGCGCAACCCTTGTCTTTAGTTGCCATCAGGTT |
| | | ATGCTGGGCACTCTAGAGAGACTGCCGAGGACAACTCGGAGGAAGGTGGGG |
| | | ATGACGTCAAATCATCATGCCCCTTATGCTTAGGGCTACACACGTGCTACAAT |
| | | GGCTGTTACAGAGGGTAGCCAAACCGTGAGGTGGAGCCAATCCCATAAAAAC |
| | | AGTCTAAGTTCGGATTGTAGGCTGAAACTCGCCTACATGAAGCTGGAGTTACT |
| | | JAGTAATCGCAGATCAGCATGCTGCGGTGAATGCGTTCCCGGGTCTTGTACACA |
| | | CCGCCCGTCACACCATGGGAGCTGGGGGGGCCCAAAGTCAACTATCTAACTG |
| | | CAAAGAGGAAGTTGCCTAAGGCAAAACCAGTGACTGGGGTGAAGTCGTAACA |
| | | AGGTAGCC |
| Fusobacterium | COT-169 | >169QC027 \| Fusobacterium sp. \| COT-169 \| Clone QC027 \| JN713334 \| 59 \| |
| sp. COT-169 | | GATGAACGCTGACAGAATGCTTAACACATGCAAGTCAACTTGAACGTGGGTTT |
| (SEQ ID NO.: 12) | | TTAACTTAGGTTTGGGTGGCGGACGGGTGAGTAACGCGTAAGGAACTTGCCT |
| | | CACAGATAGGGACAACATTTAGAAATGAATGCTAATACCTAATATTATGAAAA |
| | | TAGGGCATCCTATAATTATGAAAGCTAAATGCGCTGTGAGAGAGCTTTGCGTC |
| | | CCATTAGCTAGTTGGAGAGGTAACGGCTCACCAAGGCGATGATGGGTAGCCG |
| | | GCCTGAGAGGGTGAACGGCCACAAGAGGACTGAGACACGGCCCTTACTCCTA |
| | | CGGGAGGCAGCAGTGGGGAATATTGGACAATGGACCAAGAGTCTGATCCAG |
| | | CAATTCTGTGTGCACGATGAAGTTTTTCGGAATGTAAAGTGCTTTCAGTTGGGA |
| | | AGAAAAGAATGACGGTACCGACAGAAGAAGTGACGGCTAAATACGTGCCAG |
| | | CAGCCGCGGTAATACGTATGTCACGAGCGTTATCCGGATTTATTGGGCGTAAA |
| | | GTGCGTCTAGGTGGTTATGTAAGTCTGATGTGAAAATGCAGGGCTCAACTCTG |
| | | TATTGCGTTGGAAACTGTGTAACTAGAGTACTGGAGAGGTAAGCGGAACTAC |
| | | AAGTGTAGAGGTGAAATTCGTAGATATTTGTAGGAATGCCGATGGGGAAGCC |
| | | AGCTTACTGGACAGATACTGACGCTAAAGCGCGAAAGCGTGGGTAGCAAACA |
| | | GGATTAGATACCCTGGTAGTCCACGCCGTAAACGATGATTACTAGGTGTTGGG |
| | | GGTCGAACCTCAGCGCCCAAGCAAACGCGATAAGTAATCCGCCTGGGGAGTA |
| | | CGTACGCAAGTATGAAACTCAAAGGAATTGACGGGGACCCGCACAAGCGGT |
| | | GGAGCATGTGGTTTAATTCGACGCAACGCGAGGAACCTTACCAGCGTTTGAC |
| | | ATCTTAGGAATGAGATAGAGATATTTCAGTGTCCCTTCGGGGAAACCTAAAGA |
| | | CAGGTGGTGCATGGCTGTCGTCAGCTCGTGTCGTGAGATGTTGGGTTAAGTCC |
| | | CGCAACGAGCGCAACCCTTTTCGTATGTTACCATCATTAAGTTGGGGACTCAT |
| | | GCGATACTGCCTGCGATGAGCAGGAGGAAGGTGGGGATGACGTCAAGTCAT |
| | | CATGCCCCTTATACGCTGGGCTACACACGTGCTACAATGGGTAGAACAGAGA |
| | | GTTGCAAAGCCGTGAGGTGGAGCTAATCTCAGAAAACTATTCTTAGTTCGGAT |
| | | TGTACTCTGCAACTCGAGTACATGAAGTTGGAATCGCTAGTAATCGCGAATCA |
| | | GCAATGTCGCGGTGAATACGTTCTCGGGTCTTGTACACACCGCCCGTCACACC |
| | | ACGAGAGTTGGTTGCACCTGAAGTAGCAGGCCTAACCGTAAGGAGGGATGCT |
| | | CCGAGGGTGTGATTAGCGATTGGGGTGAAGTCGTAACAAGGT |
| Fusobacterium | COT-189 | >189QD044 \| Fusobacterium sp. \| COT-189 \| Clone QD044 \| JN713356 \| 74 \| |
| sp. | | GATGAACGCTGACAGAATGCTTAACACATGCAAGTCAACTTGAACTTCGGTTT |
| COT-189 | | GGGTGGCGGACGGGTGAGTAACGCGTAAAGAACTTGCCTCACAGTCTAGGAC |
| (SEQ ID NO.: 13) | | AACATCTGGAAACGGATGCTAAAACTGGATATTATGAACTATGCGCATGCATA |
| | | GATTATGAAAGCTATATGCGCTGTGAGAGAGTTTTGCGTCCCATTAGCTAGTT |
| | | GGAGAGGTAACGGCTCACCAAGGCGATGATGGGTAGCCGGCCTGAGAGGGC |
| | | GAACGGCCACAAGGGGACTGAGACACGGCCCTTACTCCTACGGGAGGCAGC |
| | | AGTGGGGAATATTGGACAATGGACCAAGAGTCTGATCCAGCAATTCTGTGTG |
| | | CACGATGAAGTTTTTCGGAATGTAAAGTGCTTTCAGTTGGGAAGAAGAAAGTG |
| | | ACGGTACCAACAGAAGAAGCGACGGCTAAATACGTGCCAGCAGCCGCGGTA |
| | | ATACGTATGTCGCAAGCGTTATCCGGATTTATTGGGCGTAAAGCGCGTCTAGG |
| | | CGGCTAAATAAGTCTGATGTGAAAATGCGGGGCTCAACTCCGTATTGCGTTGG |
| | | AAACTGTATAGCTAGGGTACTGGAGAGGTAAGCGGAACTACAAGTGTAGAGG |
| | | TGAAATTCGTAGATATTTGTAGGAATGCCGATGGGGAAGCCAGCTTACTGGAC |
| | | AGATACTGACGCTAAAGCGCGAAAGCGTGGGTAGCAAACAGGATTAGATACC |
| | | CTGGTAGTCCACGCCGTAAACGATGATTACTAGGTGTTGGGGGTCGAACCTCA |
| | | GCGCCCAAGCAAACGCGATAAGTAATCCGCCTGGGGAGTACGTACGCAAGTA |
| | | TGAAACTCAAAGGAATTGACGGGGACCCGCACAAGCGGTGGAGCATGTGGTT |
| | | TAATTCGACGCAACGCGAGGAACCTTACCAGCGTTTGACATCCTACTAAGTAC |
| | | TCAGAGATGAGTATGTGCTCTTTCGGGAGAAAGTAGAGACAGGTGGTGCATG |
| | | GCTGTCGTCAGCTCGTGTCGTGAGATGTTGGGTTAAGTCCCGCAACGAGCGC |
| | | AACCCCTTTCGTATGTTACCATCATTAAGTTGGGGACTCATGCGATACTGCCTG |
| | | CGATGAGCAGGAGGAAGGTGGGGATGACGTCAAGTCATCATGCCCCTTATAC |
| | | GCTGGGCTACACACGTGCTACAATGGGTAGAACAGAGAGCAGCGAAGCTGT |
| | | GAAGTGGAGCAAATCTCAGAAAACTATTCTTAGTTCGGATTGTACTCTGCAAC |
| | | TCGAGTACATGAAGTTGGAATCGCTAGTAATCGCAAATCAGCAATGTTGCGGT |
| | | GAATACGTTCTCGGGTCTTGTACACACCGCCCGTCACACCACGAGAGTTGGTT |
| | | GCACCTGAAGTAACAGGCCTAACCGTAAGGAGGGATGTTCCGAGGGTGTGAT |
| | | TAGCGATTGGGGTGAAGTCGTAACAAGGT |
| Gemella | COT-089 | >089OE022 \| Gemella palaticanis \| COT-089 \| Clone OE022 \| JN713253 \| 26 \| |
| palaticanis COT- | | GACGAACGCTGGCGGCGTGCCTAATACATGCAAGTCGAGCGGAGTTGAATAA |
| 089 | | GTGCTTGCACTAATTCAACTTAGCGGCGAACGGGTGAGTAACACGTAAAGAA |
| (SEQ ID NO.: 14) | | CCTGCCTCATAGACTGGGACAACTACTGGAAACGGTAGCTAATACCGGATAA |
| | | CATATTTTTCTGCATGGAAGAATATTGAAAGTCGGTCATGCTGACACTATGAG |
| | | ATGGCCTTGCGGTGCATTAGCTAGTTGGTAGGGTAAAGGCCTACCAAGGCGA |
| | | CGATGCATAGCCGACCTGAGAGGGTGATCGGCCACACTGGGACTGAGACAC |
| | | GGCCCAGACTCCTACGGGAGGCAGCAGTAGGGAATCTTCCGCAATGGACGA |
| | | AAGTCTGACGGAGCAACGCCGCGTGAGTGAAGAAGGATTTCGGTTCGTAAAA |
| | | CTCTGTTGTTAGGGAAGAATGATTGTATAGTAACTATATACAGTAGAGACGGT |
| | | ACCTAACCAGAAAGCCACGGCTAACTACGTGCCAGCAGCCGCGGTAATACGT |
| | | AGGTGGCAAGCGTTGTCCGGAATTATTGGGCGTAAAGCGCGCGCAGGTGGTT |
| | | TAAAAAGTCTGATGTGAAAGCCCACGGCTCAACCGTGGAAGGTCATTGGAAA |
| | | CTGTTAAACTTGAGTGCAGGAGAGAAAAGTGGAATTCCTAGTGTAGCGGTGA |
| | | AATGCGTAGAGATTAGGAGGAACACCAGTGGCGAAGGCGGCTTTTTGGCCTG |
| | | TAACTGACACTGAGGCGCGAAAGCGTGGGGAGCAAACAGGATTAGATACCCT |
| | | GGTAGTCCACGCCGTAAACGATGAGTGCTAAGTGTTGGAGTCATAAGACTTCA |
| | | GTGCTGCAGCTAACGCATTAAGCACTCCGCCTGGGGAGTACGGTCGCAAGAC |
| | | TGAAACTCAAAGGAATTGACGGGGACCCGCACAAGCGGTGGAGTATGTGGTT |
| | | TAATTCGAAGCAACGCGAAGAACCTTACCAAGTCTTGACATACAGTGAAGATT |
| | | TAAGAGATTAGATTGTTTATACCTATGGTATAACACTGATACAGGTGGTGCAT |
| | | GGTTGTCGTCAGCTCGTGTCGTGAGATGTTGGGTTAAGTCCCGCAACGAGCGC |
| | | AACCETTATATCTAGTTGCCAGCAGTAAGATGGGGACTCTAGATAGACTGCCA |
| | | GTGATAAACTGGAGGAAGGTGGGGATGACGTCAAATCATCATGCCCCTTATG |
| | | ACTTGGGCTACACACGTACTACAATGGATAGGAACAAAGAGAAGCAACCTCG |
| | | TAAGAGCAAGCCAACCTCATAAAACTATTCTCAGTTCGGATTGTAGTCTGCAA |
| | | CTCGACTACATGAAGCTGGAATCGCTAGTAATCGCGAATCAGAATGTCGCGGT |
| | | GAATACGTTCCCGGGTCTTGTACACACCGCCCGTCACACCACGGGAGTTTGTA |
| | | ACACCCGAAGACGGTGGCCTAACCGCAAGGAGGGAGCCGGTCACGGTGGGA |
| | | TAGATGATTGGGGTGAAGTCGTAACAAGGTAGCCGTATCGGAAGGTGCGGCT |
| | | GGATCACCTCCTT |
| Granulicatella | COT-095 | >095PO044 \| Granulicatella sp. \| COT-095 \| Clone PO044 \| JN713259 \| 72 \| |
| sp. COT-095 | | GACGAACGCTGGCGGCGTGCCTAATACATGCAAGTCGAACGAACGGAGAAA |
| (SEQ ID NO.: 15) | | GAAGCTTGCTTTTTTCAAAGTTAGTGGCGAACGGGTGAGTAACACGTGGGTAA |
| | | CCTGCCCATCAGAGGGGGATAACATTCGGAAACGGATGCTAAAACCGCATAG |
| | | GTTCCTTAGTTGCATGACTGAGGAAGGAAAAGAGGCGTAAGCTTCTGCTGGT |
| | | GGATGGACCCGCGGTGCATTAGCTAGTTGGTGAGGTAACGGCTCACCAAGGC |
| | | GATGATGCATAGCCGACCTGAGAGGGTGATCGGCCACATTGGGACTGAGACA |
| | | CGGCCCAAACTCCTACGGGAGGCAGCAGTAGGGAATCTTCCGCAATGGACGC |
| | | AAGTCTGACGGAGCAACGCCGCGTGAGTGAAGAAGGTTTTCGGATCGTAAAA |
| | | CTCTGTTGTTAGAGAAGAATAAGCATAATAGTAACTGATTATGCCGTGACGGT |
| | | ATCTAACCAGAAAGCCACGGCTAACTACGTGCCAGCAGCCGCGGTAATACGT |
| | | AGGTGGCAAGCGTTGTCCGGATTTATTGGGCGTAAAGCGAGCGCAGGCGGTT |
| | | CCTTAAGTCTGATGTGAAAGCCCACGGCTCAACCGTGGAGGGTCATTGGAAA |
| | | CTGGGGAACTTGAGTGCAGAAGAGGAGAGTGGAATTCCATGTGTAGCGGTGA |
| | | AATGCGTAGATATATGGAGGAACACCAGTGGCGAAGGCGACTCTCTGGTCTG |
| | | TAACTGACGCTGAGGCTCGAAAGCGTGGGTAGCAAACAGGATTAGATACCCT |
| | | GGTAGTCCACGCCGTAAACGATGAGTGCTAAGTGTTGGAGGGTTTCCGCCCTT |
| | | CAGTGCTGCAGTTAACGCATTAAGCACTCCGCCTGGGGAGTACGACCGCAAG |
| | | GTTGAAACTCAAAGGAATTGACGGGGACCCGCACAAGCGGTGGAGCATGTG |
| | | GTTTAATTCGAAGCAACGCGAAGAACCTTACCAAGTCTTGACATCCTTTGACC |
| | | ACTCTAGAGATAGAGCTTTCCCTTCGGGGACAAAGTGACAGGTGGTGCATGG |
| | | TTGTCGTCAGCTCGTGTCGTGAGATGTTGGGTTAAGTCCCGCAACGAGCGCAA |
| | | CCCTTATTACTAGTTGCCAGCATTCAGTTGGGCACTCTAGTGAGACTGCCGGT |
| | | GACAAACCGGAGGAAGGTGGGGATGACGTCAAATCATCATGCCCCTTATGAC |
| | | TTGGGCTACACACGTGCTACAATGGATGGTACAACGAGCAGCGAACTCGCGA |
| | | GGGTAAGCGAATCTCTTAAAGCCATTCTCAGTTCGGATTGTAGGCTGCAACTC |
| | | GCCTACATGAAGCCGGAATCGCTAGTAATCGCGGATCAGCACGCCGCGGTGA |
| | | ATACGTTCCCGGGTCTTGTACACACCGCCCGTCACACCACGAGAGTTTGTAAC |
| | | ACCCAAAGTCGGTGAGGTAACCTATTGGAGCCAGCCGCCTAAGGTGGGATAG |
| | | ATGATTGGGGTGAAGTCGTAACAAGGTAGCCGTATCGGAAGGTGCGGCTGGA |
| | | TCACCTCCTT |
| Helcococcus sp. | COT-069 | >069OF006 \| Helcococcus sp. \| COT-069 \| Clone OF006 \| JN713234 \| 94 \| |
| COT-069 | | GACAAACGCTGGCGGCGTGCATAACACATGCAAGTCGAACGAAATCTACATG |
| (SEQ ID NO.: 16) | | ATTTAGTGGCGAACGGGTGAGTAACACGTGAGAAACCTGCCTTACACAGAGG |
| | | GATAGCCGTTGGAAACGACGATTAATACCTCATGACACCGTGACATCGCATG |
| | | ATGATGCGGTCAAAGATTTATCGGTGTAAGATGGTCTCGCGCCTGATTAGCTG |
| | | GTTGGTGGGGTAACGGCCTACCAAGGCGACGATCAGTAGCCGATCTGAGAGG |
| | | GTGGACGGCCACATTGGGACTGAGACACGGCCCAAACTCCTACGGGAGGCA |
| | | GCAGTGGGGGATTTTGCACAATGGGGGGAACCCTGATGCAGCGACGCCGCG |
| | | TGAACGAAGAAGGCCTTCGGGTTGTAAAGTTCTGTCCTTGGTGAAGATAATGA |
| | | CAGTAACCAAGAAGAAAGCCCCGGCTAAATACGTGCCAGCAGCCGCGGTAAT |
| | | ACGTATGGGGCGAGCGTTGTCCGGAATTATTGGGCGTAAAGGGTACGTAGGC |
| | | GGTTATTTAAGTCCATATTAAAAGGCATTGGCTCAACCAATGTAAGGTGTGGA |
| | | AACTGGATAACTTGAGTAGATGAGGGGAAAGTGGAATTCCAAGTGTAGCGGT |
| | | GAAATGCGTAGATATTTGGAGGAATACCGGTGGCGAAGGCGACTTTCTGGAA |
| | | TCTAACTGACGCTGAGGTACGAAAGCGTGGGGAGCAAACAGGATTAGATACC |
| | | CTGGTAGTCCACGCAGTAAACGATGAGTGCTAGGTGTCGGGGGTCAAACCTC |
| | | GGCGCCGCCGCTAACGCATTAAGCACTCCGCCTGGGGAGTACGTACGCAAGT |
| | | ATGAAACTCAAAGGAATTGACGCGGACCCGCACAAGCAGCGGAGCATGTGG |
| | | TTTAATTCGAAGCAACGCGAAGAACCTTACCAGGGCTTGACATATACAGGAAT |
| | | GTGCTAGAGATAGTACAGTCTTTTCGAAGACTTGTATACAGGTGGTGCATGGT |
| | | TGTCGTCAGCTCGTGTCGTGAGATGTTGGGTTAAGTCCCGTAACGAGCGCAAC |
| | | CCCTATCTTTAGTTACCATCAGTAAGGCTGGGGACTCTAGAGAGACTGCCGGT |
| | | GATAAACCGGAGGAAGGTGGGGATGACGTCAAATCATCATGCCCTATATGTC |
| | | CTGGGCTACACACGTGCTACAATGGTCTGAACAAAGTGCAGCGAACTCGTGA |
| | | GGGTAAGCGAATCACAGAAAACAGATCTCAGTTCGGATTGTAGGCTGCAACT |
| | | CGCCTACATGAAGTCGGAGTTGCTAGTAATCGTGAATCAGAATGTCACGGTGA |
| | | ATGCGTTCCCGGGTCTTGTACACACCGCCCGTCACACCATGGGAGTTGGCAAT |
| | | ACCCGAAGTCGTCGAGCTAACCGTAAGGAGGCAGACGCCGAAGGTAGGGTT |
| | | AATGACTGGGGTGAAGTCGTAACAAGGTAGCCGTATCGGAAGGTGCGGCTGG |
| | | ATCACCTCCTT |
| Lachnospiraceae | COT-099 | >099PO010 \| Lachnospiraceae XIVa [G-4] sp. \| COT-099 \| Clone PO010 \| |
| XIVa [G-4] sp. | | JN713263 \| 30 \| |
| COT-099 | | GATGAACGCTGGCGGCGTGCCTAACACATGCAAGTCGAACGAAGCGATGAA |
| (SEQ ID NO.: 17) | | GCGGAAGTTTTCGGACGGAAGCATCATTGACTTAGTGGCGGACGGGTGAGTA |
| | | ACGCGTGGGTAACCTGCCTCATACAGGGGGATAACAGTGGGAAACGACTGCT |
| | | AATACCCCATAAGCGCACGGCAGCAAAAGCTGCAGGGCGAAAAGCGAATAC |
| | | GGTATGAGATGGGCCCGCGTCCGATTAGCCAGTTGGCAGGGTAAGAGCCTAC |
| | | CAAAGCGACGATCGGTAGCCGGCTTGAGAGAGTGACCGGCCACATTGGGACT |
| | | GAGACACGGCCCAAACTCCTACGGGAGGCAGCAGTGGGGGATATTGCACAA |
| | | TGGGGGGAACCCTGATGCAGCGACGCCGCGTGAAGGAAGAAGTATTTCGGT |
| | | ATGTAAACTTCTATCAGCAGGGAAGAAAATGACGGTACCTGACTAAGAAGCA |
| | | CCGGCTAAATACGTGCCAGCAGCCGCGGTAATACGTATGGTGCAAGCGTTAT |
| | | CCGGAATTACTGGGTGTAAAGGGAGCGTAGGCGGATAGGTAAGTCAGATGTG |
| | | AAAGACCGGGGCTCAACTCTGGGGCTGCATTTGAAACTATCTAACTAGAGTAC |
| | | AGGAGAGGTAAGCGGAATTCCTAGTGTAGCGGTGAAATGCGTAGATATTAGG |
| | | AGGAACATCGGTGGCGAAGGCGGCTTACTGGACTGAAACTGACGCTGAGGCT |
| | | CGAAAGCGTGGGGAGCAAACAGGATTAGATACCCTGGTAGTCCACGCTGTAA |
| | | ACGATGAATACTAGGTGTTGGTGCCCATGGGGCATCGGTGCCGCAGCTAACG |
| | | CAATAAGTATTCCACCTGGGGAGTACGTTCGCAAGAATGAAACTCAAAGGAA |
| | | TTGACGGGGACCCGCACAAGCGGTGGAGCATGTGGTTTAATTCGAAGCAACG |
| | | CGAAGAACCTTACCAAGTCTTGACATCCTTCTGACCGGCCCGTAACGGGGCCT |
| | | TTCCTTCGGGACAGAAGAGACAGGTGGTGCATGGTTGTCGTCAGCTCGTGTCG |
| | | TGAGATGTTGGGTTAAGTCCCGCAACGAGCGCAACCCCTATCCGAAGTAGCC |
| | | AGCAGGTAGAGCTGGGCACTCTGCGGAGACAGCCGGGGACAACCCGGAGGA |
| | | AGGCGGGGATGACGTCAAATCATCATGCCCCTTATGATTTGGGCTACACACGT |
| | | GCTACAATGGCGTATACAAAGGGAAGCGAGACGGTGACGTTAAGCAAATCTC |
| | | AAAAAAGGCGCCCCAGTTCGGATTGTAGTCTGCGACTCGACTACATGAAGCC |
| | | GGAATCGCTAGTAATCGCAGGTCAGCATACTGCGGTGAATACGTTCCCGGGT |
| | | CTTGTACACACCGCCCGTCACACCATGGGAGTCAGCAACGCCCGAAGTCAGT |
| | | GACCCAACCGTAAGGAGGGAGCTGCCGAAGGCGGGGCGGATGACTGGGGTG |
| | | AAGTCGTAACAAGGTAGCCGTATCGGAAGGTGCGGCTGGATCACCTCCTT |
| Lachnospiraceae | COT-024 | >024OB020 \| Lachnospiraceae XIVa [G-5] sp. \| COT-024 \| Clone OB020 |
| XIVa [G-5] sp. | | JN713186 \| 77 \| |
| COT-024 | | GATGAACGCTGGCGGCGTGCCTAACACATGCAAGTCGAACGAAGCATATAGA |
| (SEQ ID NO.: 18) | | AGGAAGTTTTCGGATGGAATTTTATATGACTTAGTGGCGGACGGGTGAGTAAC |
| | | GCGTGGATAATCTGCCTTACACAGCGGGATAACAAATGGAAACAGTTGCTAA |
| | | TACCGCATAAGCACACAGTATCGCATGATACAGGGTGAAAAGATTTATCGGT |
| | | GTAAGATGAGTCCGCGTCCCATTAGCTGGTTGGTGAGGTAAAAGCTTACCAA |
| | | GGCGACGATGGGTAGCCGGCCTGAGAGGGTGAACGGCCACATTGGGACTGA |
| | | GACACGGCCCAGACTCCTACGGGAGGCAGCAGTGGGGGATATTGCACAATG |
| | | GGCGAAAGCCTGATGCAGCGACGCCGCGTGAAGGAAGAAGTATTTCGGTATG |
| | | TAAACTTCTATCAGCAGGGAAGAAAATGACGGTACCTGACTAAGAAGCCCTG |
| | | GCTAAATACGTGCCAGCAGCCGCGGTAATACGTATGGGGCAAGCGTTATCCG |
| | | GATTTACTGGGCGTAAAGGGAGCGTAGGCGGCATGGCAAGTCTGATGTGAAA |
| | | GACCGGGGCTTAACTCCGGGGTTGCATTGGAAACTGTCAAGCTAGAGTGTCG |
| | | GAGAGGTAAGCGGAATTCCTGGTGTAGTGGTGAAATGCGTAGATATTAGGAG |
| | | GAACATCGGTGGCGAAGGCGGCTTACTGGACGATAACTGACGCTCAGGCTCG |
| | | AAAGCGTGGGGAGCAAACAGGATTAGATACCTTGGTAGTCCACGCCGTAAAC |
| | | GATGAATACTAGGTGTTGGGGAGCTAAGCTCTTCGGTGCCGCAGCAAACGCA |
| | | ATAAGTATTCCGCCTGGGGAGTACGTTCGCAAGAATGAAACTCAAAGGAATT |
| | | GACGGGGACCCGCACAAGCGGTGGAGCATGTGGTTTAATTCGAAGCAACGC |
| | | GAAGAACCTTACCAGGCCTTGACATCGATCCGACTAGGAAGTAATGTTTCTAT |
| | | CTCTTCGGAGCGGAGAAGACAGGTGGTGCATGGTTGTCGTCAGCTCGTGTCG |
| | | TGAGATGTTGGGTTAAGTCCCGCAACGAGCGCAACCCTTATCCTTAGTAACCA |
| | | GCGAGTGAAGTCGGGGACTCTAGGGAGACTGCCGGGGATGACCCGGAGGAA |
| | | GGTGGGGACGACGTCAAATCATCATGCCCCTTACGGTCTGGGCTACACACGT |
| | | GCTACAATGGGACATACAAAGGGAGGCGAAGGAGTGATCCGGAGCAAACCC |
| | | CAAAAAGTGTTCCTCAGTTCGGATTGTAGGCTGCAACTCGCCTACATGAAGCC |
| | | GGAATCGCTAGTAATCGCAAATCAGAATGTTGCGGTGAATACGTTCCCGGGTC |
| | | TTGTACACACCGCCCGTCACACCATGGGAGTCGAAAATGCCCGAAGTCAGTG |
| | | ACCCAACCGCAAGGAGGGAGCTGCCGAAGGCAGGTTTGGTGACTGGGGTGA |
| | | AGTCGTAACAAGGTAGCCGTATCGGAAGGTGCGGCTGGATCACCTCCTT |
| Leptotrichia sp. | COT-345 | > 3451J034 \| Leptotrichia sp. \| COT-345 \| Clone 1J034 \| JN713514 \| 7 \| |
| COT-345 | | GATGAACGCTGACAGAATGCTTAACACATGCAAGTCGATGGGGAAGTAGTAC |
| (SEQ ID NO.: 19) | | TTGTACTATGGTAACCACGGCGGACGGGTGAGTAACGCGTAAAGAACTTACC |
| | | TTTAGGACAGGGATAACAGAGGGAAACTTCTGATAAGACTTGATATAATTATG |
| | | TAGTTGCATGACTGTATAATGAAAAGGGATGCCTAGAGAGAGCTTTGCGTCCT |
| | | ATTAGTTAGTTGGTGTGGTAAAGGCATACCAAGGCGATGATAGGTAGCCGGC |
| | | CTGAGAGGGTGGACGGCCACAAGGGGACTGAGATACGGCCCTTACTCTTACG |
| | | GGAGGCAGCAGTGGGGAATATTGGACAATGGAGGAGACTCTGATCCAGCAA |
| | | TTCTGTGTGCACGAAGAAGGTTTTCGGATTGTAAAGTGCTTTCAACAGGGAAG |
| | | AAGAAAGTGACGGTACCTGTAGAAGAAGCGACGGCTAAATACGTGCCAGCA |
| | | GCCGCGGTAATACGTATGTCGCGAGCGTTATCCGGAATTATTGGGCATAAAG |
| | | GGCATCTAGGCGGCATAGCAAGTCAGGGGTGAAAACTTGTGGCTCAACCATA |
| | | AGCTTGCCTTTGAAACTGTGATGCTAGAGTATTGGAAAGGTGGGCGGAACTAC |
| | | ACGAGTAGAGGTGAAATTCGTAGATATGTGTAGGAATGCCGATGATGAAGAT |
| | | AGCTCACTGGACGATAACTGACGCTGAAGTGCGAAAGCTAGGGGAGCGAAC |
| | | AGGATTAGATACCCTGGTAGTCCTAGCTGTAAACGATGATCACTGGGTGTGGG |
| | | CAGGAAGACTGTCTGTGCCGAAGCAAATGCGATAAGTGATCCGCCTGGGGAG |
| | | TACGACCGCAAGGTTGAAACTCAAAGGAATTGACGGGGACCCGCACAAGCG |
| | | GTGGAGCATGTGGTTTAATTCGACGCAACGCGAGGAACCTTACCAGATCTTGA |
| | | CATCCTCAGAAGATAGTAGAAGTACTATTGTGCCTTTGGGAACTGAGAGACAG |
| | | GTGGTGCATGGCTGTCGACAGCTCGTGTCGTGAGATGTTGGGTTAAGTCCCGC |
| | | AACGAGCGCAACCCTTATCGTTAGTTACCATCATTAAGTTGGGGACTCTAGCG |
| | | AGACTGCCTGCGAAGAGCAGGAGGAAGGTGGGGATGACGTCAAGTCATCAT |
| | | GCCCCTTATGATCTGGGCTACACACGTGCTACAATGGGCAGTACAAAGAGGG |
| | | GCGAGATGGTGACATGGAGCAAATCTTGAAAGCTGTTCTAAGTTCGGATTGAA |
| | | GTCTGCAACTCGACTTCATGAAGTTGGAATCGCTAGTAATCGCAAATCAGCAA |
| | | TGTTGCGGTGAATACGTTCTCGGGTCTTGTACACACCGCCCGTCACACCACGA |
| | | GAGTTGTTTGCACCTGAAGTTACTGGTCTAACCGTAAGGAGGAAGGTACCTAA |
| | | GGTGTGAATAGTGATTGGGGTGAAGTCGTAACAAGGT |
| Moraxella sp. | COT-017 | >017OB023 \| Moraxella sp. \| COT-017 \| Clone OB023 \| JN713178 \| 78 \| |
| COT-017 | | ATTGAACGCTGGCGGCAGGCTTAACACATGCAAGTCGAACGATGATTACTAG |
| (SEQ ID NO.: 20) | | CTTGCTAGTATGATTAGTGGCGAACGGGTGAGTAATGCTTAGGAATCTGCCTA |
| | | TTAGTGGGGGACAACATTCCGAAAGGAATGCTAATACCGCATACGTCCTACG |
| | | GGAGAAAGCAGGGGATCTTTTGACCTTGCGCTAATAGATGAGCCTAAGTCGG |
| | | ATTAGCTAGTTGGTGGGGTAAAGGCCTACCAAGGCGACGATCTGTAGCTGGT |
| | | CTGAGAGGATGATCAGCCACACTGGGACTGAGACACGGCCCAGACTCCTACG |
| | | GGAGGCAGCAGTGGGGAATATTGGACAATGGGCGAAAGCCTGATCCAGCCA |
| | | TGCCGCGTGTGTGAAGAAGGCCTTTTGGTTGTAAAGCACTTTAAGTGGGGAG |
| | | GAAAAGCTAATAATTAATACCTATTAGCCCTGACGTTACCCACAGAATAAGCA |
| | | CCGGCTAACTCTGTGCCAGCAGCCGCGGTAATACAGAGGGTGCAAGCGTTAA |
| | | TCGGAATTACTGGGCGTAAAGCGAGCGTAGGTGGTTACTTAAGTCAGATGTG |
| | | AAAGCCCCGGGCTTAACCTGGGAACTGCATCTGATACTGGGTAACTAGAGTA |
| | | GGTGAGAGGGAAGTAGAATTCCAGGTGTAGCGGTGAAATGCGTAGAGACCT |
| | | GGAGGAATACCGATGGCGAAGGCAGCTTCCTGGCATCATACTGACACTGAGG |
| | | TTCGAAAGCGTGGGTAGCAAACAGGATTAGATACCCTGGTAGTCCACGCCGT |
| | | AAACGATGTCTACCAGTCGTTGGGTCTTTTAAAGACTTAGTGACGCAGTTAAC |
| | | GCAATAAGTAGACCGCCTGGGGAGTACGGCCGCAAGGTTAAAACTCAAATGA |
| | | ATTGACGGGGGCCCGCACAAGCGGTGGAGCATGTGGTTTAATTCGATGCAAC |
| | | GCGAAGAACCTTACCTGGTCTTGACATAGTAGGAATCCTGCAGAGATGCGGG |
| | | AGTGCCTTCGGGAATCTACATACAGGTGCTGCATGGCTGTCGTCAGCTCGTGT |
| | | CGTGAGATGTTGGGTTAAGTCCCGCAACGAGCGCAACCCTTTTCCTTAGTTAC |
| | | CAGCGGGTTATGCCGGGAACTCTAAGGATACTGCCAGTGACAAACTGGAGGA |
| | | AGGCGGGGACGACGTCAAGTCATCATGGCCCTTACGACCAGGGCTACACACG |
| | | TGCTACAATGGTAGGTACAAAGGGTTGCTACACAGCGATGTGATGCTAATCTC |
| | | AAAAAGCCTATCGTAGTCCGGATTGGAGTCTGCAACTCGACTCCATGAAGTCG |
| | | GTATCGCTAGTAATCGCAGATCAGAATGCTGCGGTGAATACGTTCCCGGGCCT |
| | | TGTACACACCGCCCGTCACACCATGGGAGTTGATCTCACCAGAAGTGGGTAG |
| | | CCTAACGCAAGAGGGCGCTCACCACGGTGGGGTCGATGACTGGGGTGAAGT |
| | | CGTAACAAGGTAGCCGTAGGGGAACCTGCGGCTGGATCACCTCCTT |
| Moraxella sp. | COT-018 | >018OB065 \| Moraxella sp. \| COT-018 \| Clone OB065 \| JN713179 \| 94 \| |
| COT-018 | | ATTGAACGCTGGCGGCAGGCTTAACACATGCAAGTCGAACGGAAGTTACTAG |
| (SEQ ID NO.: 21) | | CTTGCTAGTAACTTTAGTGGCGAACGGGTGAGTAATATTTAGGAATCTGCCTC |
| | | ATAACGGGGGATAGCTCGGGGAAACTCGAATTAATACCGCATACACCCTACG |
| | | GGGGAAAGGTGGCGCAAGCTGCCGATATGAGATGAGCCTAAATCAGATTAGC |
| | | TAGATGGTGGGGTAAAGGCCTACCATGGCGACGATCTGTAACTGGTCTGAGA |
| | | GGATGATCAGTCACACCGGAACTGAGACACGGTCCGGACTCCTACGGGAGG |
| | | CAGCAGTGGGGAATATTGGACAATGGGCGAAAGCCTGATCCAGCCATGCCGC |
| | | GTGTGTGAAGAAGGCCATAAGGTTGTAAAGCACTTTAAGCAGGGAGGAAGAA |
| | | CTTTTAGTTAATAGCTAAAAGTGGTGACGTTACCTGCAGAATAAGCACCGGCT |
| | | AACTCTGTGCCAGCAGCCGCGGTAATACAGAGGGTGCGAGCGTTAATCGGAA |
| | | TTACTGGGCGTAAAGCGAGCGTAGGTGGTTGATTAAGTCAGCTGTGAAATCCC |
| | | CGGGCTTAACCTGGGAAAGTCAGCTGATACTGGTTAACTAGAGTATGTGAGA |
| | | GGAAAGTAGAATTCCAGGTGTAGCGGTGAAATGCGTAGAGATCTGGAGGAAT |
| | | ACCGATGGCGAAGGCAGCTTTCTGGCATAATACTGACACTGAGGTTCGAAAG |
| | | CGTGGGTAGCAAACAGGATTAGATACCCTGGTAGTCCACGCCGTAAACGATG |
| | | TCTACTAGCCGTTGGTTACCAAGAGTAGCAAGTGGCGCAGCAAACGCGATAA |
| | | GTAGACCGCCTGGGGAGTACGGCCGCAAGGTTAAAACTCAAATGAATTGACG |
| | | GGGGCCCGCACAAGCGGTGGAGCATGTGGTTTAATTCGATGCAACGCGAAGA |
| | | ACCTTACCTGGTCTTGACATATCTAGAATCCTACAGAGATGTGGGAGTGCCTT |
| | | CGGGAATTAGAATACAGGTGCTGCATGGCTGTCGTCAGCTCGTGTCGTGAGAT |
| | | GTTGGGTTAAGTCCCGCAACGAGCGCAACCCTTATCCTTAGTTACCATCAAGT |
| | | GAAGTTGGGGACTCTAAGGAGACTGCCAGTGACAAACTGGAGGAAGGCGGG |
| | | GACGACGTCAAGTCATCATGGCCCTTACGACCAGGGCTACACACGTGCTACA |
| | | ATGGTAGGTACAGAGGGTCGCTACACTGCGAAGTGATGCCAATCTCAAAAAG |
| | | CCTATCGTAGTCCAGATTGGAGTCTGCAACTCGACTCCATGAAGTCGGAATCG |
| | | CTAGTAATCGCGGATCAGAATGTCGCGGTGAATACGTTCCCGGGCCTTGTACA |
| | | CACCGCCCGTCACACCATGGGAGTCTATTGCACCAGAAGTAGTTAGCCTAAC |
| | | GCAAGAGGGCGATTACCACGGTGTGGTCGATGACTGGGGTGAAGTCGTAACA |
| | | AGGTAGCCGTAGGGGAACCTGCGGCTGGATCACCTCCTT |
| Neisseria | COT-016 | >016OB021 \| Neisseria animaloris \| COT-016 \| Clone OB021 \| JN713177 |
| animaloris COT- | | \| 25 \| |
| 016 | | ATTGAACGCTGGCGGCATGCTTTACACATGCAAGTCGAACGGCAGCACGGAA |
| (SEQ ID NO.: 22) | | GAGCTTGCTCTTTTGGTGGCGAGTGGCGAACGGGTGAGTAACGCATCGGAAC |
| | | GTACCGAGTAGTGGGGGATAACTGTCCGAAAGGATGGCTAATACCGCATATT |
| | | CTCTGAGGAGGAAAGCAGGGGACCTTCGGGCCTTGCGCTATTTGAGCGGCCG |
| | | ATGTCTGATTAGCTAGTTGGTGGGGTAAAGGCCTACCAAGGCGACGATCAGT |
| | | AGCGGGTCTGAGAGGATGATCCGCCACACTGGGACTGAGACACGGCCCAGA |
| | | CTCCTACGGGAGGCAGCAGTGGGGAATTTTGGACAATGGGGGGAACCCTGAT |
| | | CCAGCCATGCCGCGTGTCTGAAGAAGGCCTTCGGGTTGTAAAGGACTTTTGTC |
| | | AGGGAAGAAAAGCTTGAGGTTAATACCCTTGAGTGATGACGGTACCTGAAGA |
| | | ATAAGCACCGGCTAACTACGTGCCAGCAGCCGCGGTAATACGTAGGGTGCAA |
| | | GCGTTAATCGGAATTACTGGGCGTAAAGCGGGCGCAGACGGTTACTTAAGTC |
| | | AGATGTGAAATCCCCGGGCTCAACCTGGGAACTGCGTTTGAAACTGGGTGAC |
| | | TAGAGTATGTCAGAGGGGGGTAGAATTCCACGTGTAGCAGTGAAATGCGTAG |
| | | AGATGTGGAGGAATACCGATGGCGAAGGCAGCCCCCTGGGATAATACTGAC |
| | | GTTCATGCCCGAAAGCGTGGGTAGCAAACGGGATTAGATACCCTGGTAGTCC |
| | | ACGCCCTAAACGATGTCAATTAGCTGTTGGGGTACTTGATGCCTTAGTAGCGT |
| | | AGCTAACGCGTGAAATTGACCGCCTGGGGAGTACGGTCGCAAGATTAAAACT |
| | | CAAAGGAATTGACGGGGACCCGCACAAGCGGTGGATGATGTGGATTAATTCG |
| | | ATGCAACGCGAAGAACCTTACCTGGTCTTGACATGTACGGAATCCTCCAGAGA |
| | | CGGAGGAGTGCCTTCGGGAACCGTAACACAGGTGCTGCATGGCTGTCGTCAG |
| | | CTCGTGTCGTGAGATGTTGGGTTAAGTCCCGCAACGAGCGCAACCCTTGTCAT |
| | | TAGTTGCCATCATTAAGTTGGGCACTCTAATGAGACTGCCGGTGACAAGCCGG |
| | | AGGAAGGTGGGGATGACGTCAAGTCCTCATGGCCCTTATGACCAGGGCTTCA |
| | | CACGTCATACAATGGTCGGTACAGAGGGTAGCCAAGCCGCGAGGTGGAGCC |
| | | AATCCCAAAAAACCGATCGTAGTCCGGATTGCACTCTGCAACTCGAGTGCATG |
| | | AAGTCGGAATCGCTAGTAATCGCAGGTCAGCATACTGCGGTGAATACGTTCCC |
| | | GGGTCTTGTACACACCGCCCGTCACACCATGGGAGTGGGGGATACCAGAAGT |
| | | AGGTAGGGTAACCGTAAGGAGCCCGCTTACCACGGTATGCTTCATGACTGGG |
| | | GTGAAGTCGTAACAAGGTAGCCGTAGGGGAACCTGCGGCTGGATCACCTCCT |
| | | T |
| Odoribacter | COT-084 | >0840C024 \| Odoribacter denticanis \| COT-084 \| Clone OC024 \| JN713247 |
| denticanis COT- | | \| 16 \| |
| 084 | | GATGAACGCTAGCGATAGGCTTAACACATGCAAGTCGAGGGGTAACAGGCG |
| (SEQ ID NO.: 23) | | GTAGCAATACTGTGCTGACGACCGGCGGATGGGTGAGTAACGCGTATGCAAT |
| | | CTACCTTTTACCCGGGGATAGCCCATGGAAACGTGGATTAATACCCGATGCAT |
| | | TTCTTTTGTGGCCTCATGAAGGGAATAAAGATTTATCGGTAAGAGATGAGCAT |
| | | GCGTTCCATTAGGAAGTTGGTAAGGTAACGGCTTACCAATCCGATGATGGATA |
| | | GGGGTTCTGAGAGGAAGGTCCCCCACACTGGAATTGAGAAACGGTCCAGACT |
| | | CCTACGGGAGGCAGCAGTGAGGAATATTGGTCAATGGTCGTAAGACTGAACC |
| | | AGCCAAGTCGCGTGAAGGAAACTGCCCTATGGGTTTTCAACTTCTTTTGTCAG |
| | | GGAAGAATAAGGAGGATTCAATTCTCCGATGCCGGTACCTGACGAATAAGGA |
| | | TCGGCTAACTCCGTGCCAGCAGCCGCGGTAATACGGAGGATCCGAGCGTTAT |
| | | CCGGATTTATTGGGTTTAAAGGGTGCTTAGGCGGCAAATTAAGTTAGTGGTTA |
| | | AATAGTTCGGCTCAACCGGATTTCGCCATTAAAACTGATATGCTAGAGATTAA |
| | | ACGAGGTAGGCGGAATAAGTTAAGTAGCGGTGAAATGCATAGATATAACTTA |
| | | GAACACCGATAGCGAAGGCAGCTTACCAGGCTATATCTGACGCTGAATCACG |
| | | AGAGCGTGGGTATCGAACAGGATTAGATACCCTGGTAGTCCACGCCGTAAAC |
| | | GATGCTCACCGGCTCTATGCGATAAGACAGTATGGGGCTAATAGAAATAATTA |
| | | AGTGAGCCACCTGGGGAGTACGTCGGCAACGATGAAACTCAAAGGAATTGAC |
| | | GGGGGCCCGCACAAGCGGAGGAACATGTGGTTTAATTCGATGATACGCGAG |
| | | GAACCTTACCCGGGTTTAAATGTATGTTGCATTATGTAGAAATACGTATTTTCT |
| | | TCGGAACTGCATACAAGGTGCTGCATGGTTGTCGTCAGCTCGTGCCGTGAGGT |
| | | GTCGGGTTAAGTCCCATAACGAGCGCAACCCTTATGATTAGTTGCTAACGGTT |
| | | CAAGCCGAGCACTCTATTCACACTGCCACCGTAAGGTGCGAGGAAGGAGGG |
| | | GATGATGTCAAATCAGCACGGCCCTTATATCCGGGGCTACACACGTGTTACAA |
| | | TGGTCGGTACAGCGGGTTGCATTTACGTGAGTAACAGCTAATCCCAAAAATCG |
| | | GTCTCAGTTCGGATTGGAGTCTGCAACTCGACTCCATGAAGTTGGATTCGCTA |
| | | GTAATCGCACATCAGCCATGGTGCGGTGAATACGTTCCCGGGCCTTGTACACA |
| | | CCGCCCGTCAAGCCATGGGAGCTGAGGGTGCCTGAAGTTCGTAACCGCGAGG |
| | | AGCGGCCTAGGGCAAACTTGGTAACTGGGGCTAAGTCGTAACAAGGTAGCTG |
| | | TACCGGAAGGTGCGGCTGGAACACCTCCTT |
| Pasteurellaceae | COT-271 | > 271ZO044 \| Pasteurellaceae [G-2] sp. \| COT-271 \| Clone ZO044 \| JN713434 |
| sp. COT-271 | | \| 26 \| |
| (SEQ ID NO.: 24) | | ATTGAACGCTGGCGGCAGGCTTAACACATGCAAGTCGAACGGTAGCAGGAGA |
| | | AAGCTTGCTTTCTTGCTGACGAGTGGCGGACGGGTGAGTAATGCTTGGGAATC |
| | | TGGCTTATGGAGGGGGATAACTACGGGAAACTGTAGCTAATACCGCGTAATA |
| | | TCTTTGGATTAAAGAATGGGACATTAGCCATTCGCCATAAGATGAGCCCAAGT |
| | | GGGATTAGGTAGTTGGTGGGGTAAGGGCCTACCAAGCCGACGATCTCTAGCT |
| | | GGTCTGAGAGGATGACCAGCCACACTGGAACTGAGACACGGTCCAGACTCCT |
| | | ACGGGAGGCAGCAGTGGGGAATATTGCGCAATGGGGGCAACCCTGACGCAG |
| | | CCATGCCGCGTGAATGAAGAAGGCCTTCGGGTTGTAAAGTTCTTTCGGTGGTG |
| | | AGGAAGGCTTAAGTGCTAATAGTACTTAAGATTGACGTTAGCCACAGAAGAA |
| | | GCACCGGCTAACTCCGTGCCAGCAGCCGCGGTAATACGGAGGGTGCGAGCG |
| | | TTAATCGGAATGACTGGGCGTAAAGGGCACGCAGGCGGATAGTTAAGTGAGA |
| | | TGTGAAAGCCCCGGGCTTAACCTGGGAATTGCATTTCAGACTGGCAATCTAGA |
| | | GTATTTTAGGGAGGGGTAGAATTCCACGTGTAGCGGTGAAATGCGTAGAGAT |
| | | GTGGAGGAATACCGAAGGCGAAGGCAGCCCCTTGGGAATATACTGACGCTCA |
| | | TGTGCGAAAGCGTGGGGAGCAAACAGGATTAGATACCCTGGTAGTCCACGCT |
| | | GTAAACGGTGTCGATTTGGGGATTGGACTTTAAGTCTGGTGCCCGAAGCTAAC |
| | | GTGATAAATCGACCGCCTGGGGAGTACGGCCGCAAGGTTAAAACTCAAATGA |
| | | ATTGACGGGGGCCCGCACAAGCGGTGGAGCATGTGGTTTAATTCGATGCAAC |
| | | GCGAAGAACCTTACCTACTCTTGACATCCATGGAATCTTGTAGAGATACGAGA |
| | | GTGCCTTCGGGAACCATGAGACAGGTGCTGCATGGCTGTCGTCAGCTCGTGTT |
| | | GTGAAATGTTGGGTTAAGTCCCGCAACGAGCGCAACCCTTATCCTTTGTTGCC |
| | | AGCGATTAGGTCGGGAACTCAAAGGAGACTGCCAGTGATAAACTGGAGGAA |
| | | GGTGGGGATGACGTCAAGTCATCATGGCCCTTACGAGTAGGGCTACACACGT |
| | | GCTACAATGGTGCATACAGAGGGAAGCAATATGGCGACATGGAGCGAATCTC |
| | | ACAAAGTGCATCTAAGTCCGGATTGGAGTCTGCAACTCGACTCCATGAAGTCG |
| | | GAATCGCTAGTAATCGCGAATCAGAATGTCGCGGTGAATACGTTCCCGGGCC |
| | | TTGTACACACCGCCCGTCACACCATGGGAGTGGGTTGTACCAGAAGTAGATA |
| | | GCTTAACCTTCGGGAGGGCGTTTACCACGGTATGATTCATGACTGGGGTGAAG |
| | | TCGTAACAAGGTA |
| Peptococcus sp. | COT-044 | >044OD048 \| Peptococcus sp. \| COT-044 \| Clone OD048 \| JN713212 \| 113 \| |
| COT-044 | | GACAAATGCTGGCGGCATGCCTAACACATGCAAGTCGAACGGACAAGTTAGT |
| (SEQ ID NO.: 25) | | GGAAATTTTTCGGAATGGAAACTAATGCGTTAGTGGCGGATGGGTGAGTAAC |
| | | GCGTGGATAACCTGCCTCTAAATGGGGAATAACTACGAGAAATCGTAGCTAA |
| | | TACCGCATAATATCTCTTTATTGCATGATAGAGAGAGGAAAGACGGCCTTAGT |
| | | GCTGTCGTTTAGAGATGGATCCGCGTCTGATTAGCTAGTTGGTGAGGTAAGGG |
| | | CTCACCAAGGCGACGATCAGTAGCCGGCCTGAGAGGGTGAACGGCCACATT |
| | | GGGACTGAGACACGGCCCAAACTCCTACGGGAGGCAGCAGTGGGGAATCTT |
| | | CCGCAATGGGCGCAAGCCTGACGGAGCAATGCCGCGTGAGTGAAGAAGGCC |
| | | TTCGGGTTGTAAAACTCTGTCCTTATCGAAGAGAGAGAAGAGTAGTAACTGAC |
| | | TTTTCTAGGACGGTAGATGAGGAGGAAGCCCCGGCTAACTATGTGCCAGCAG |
| | | CCGCGGTAAAACATAGGGGGCAAGCGTTGTCCGGAATCACTGGGCGTAAAG |
| | | GGCGCGCAGGCGGTTTATTAAGTCAGGTGTGAAAGTTCGGGGCTCAACCCCG |
| | | TGATTGCACCTGATACTGATAGACTAGAGTGTTGGAGAGGTAAGTGGAATTCC |
| | | TAGTGTAGCGGTGGAATGCGTAGATATTAGGAGGAACACCAGTGGCGAAGGC |
| | | GACTTACTGGCCAATAACTGACGCTGAGGCGCGAAAGCGTGGGGAGCAAAC |
| | | AGGATTAGATACCCTGGTAGTCCACGCCGTAAACGATGAGTACTAGGTGTTG |
| | | GTTGTTTCGATACAATCGGTGCCGTAGTTAACACAATAAGTACTCCGCCTGGG |
| | | GAGTACGGTCGCAAGACTGAAACTCAAAGGAATTGACGGGGGCCCGCACAA |
| | | GCGGTGGAGCATGTGGTTTAATTCGACGCAACGCGAAGAACCTTACCAAGAC |
| | | TTGACATCCTCTTGACCGGATATGTAATGTATCCTTTTCTTCGGAACAAGAGAG |
| | | ACAGGTGGTGCATGGTTGTCGTCAGCTCGTGTCGTGAGATGTTGGGTTAAGTC |
| | | CCGCAACGAGCGCAACCCCTATAGTTAGTTGCCATCATTAAGTTGGGCACTCT |
| | | AGCAAGACTGCCGGTGACAAACCGGAGGAAGGTGGGGATGACGTCAAATCA |
| | | TCATGCCCCTTGTGTCTTGGGCTACACACGTGCTACAATGGTTGGTACAGAGG |
| | | GCAGCGAAGGAGTGATCTGGAGCGAATCCCAAAAAGCCAATCTCAGTTCGGA |
| | | TTGTAGGCTGCAACTCGCCTACATGAAGTCGGAATCGCTAGTAATCGCAGGTC |
| | | AGCATACTGCGGTGAATACGTACCCGGGCCTTGTACACACCGCCCGTCACAC |
| | | CACGAAAGTTGGTAACACCCGAAGTCGGTGGTCTGACCCGCAAGGGAGGAA |
| | | GCCGCCCACGGTGGGATCGATGATTGGGGTGAAGTCGTAACAAGGTAGCCGT |
| | | ATCGGAAGGTGCGGCTGGATCACCTCCTT |
| Peptostrepto- | COT-004 | >004OG039 \| Peptostreptococcaceae XI [G-1] sp. \| COT-004 \| Clone OG039 |
| coccaceae XI | | \| JN713157 \| 45 \| |
| [G-1] sp. COT-004 | | GATGAACGCTGGCGGCGTGCTTAACACATGCAAGTCGAACGGACCTGGTTTTT |
| (SEQ ID NO.: 26) | | AGCACAAATTGCTTATGCCTATCAGCATGGGCGGATGCGGACCCTTTGTCCAT |
| | | ACGAAAGAGCATCCGTCTACGCGTAAAAAGCGTGATAAACCATGCAAGCAGA |
| | | ACGGAAAGTAATGAGTGATTTGTGCTGAAAATCAGGTTAGTGGCGGACGGGT |
| | | GCGTAACACGTGGGCAACCTGCCCTGCTTTTCGGAATAACGCCGGGAAACCG |
| | | TCGCTAATGCCGAATGATATGAGAAGAAGGCATCTTCTTTTCATCAAAGAATTT |
| | | CGAAGCAGGATGGGCCCGCGCCGGATTAGCTGGTTGGTGAGGTAACGGCTC |
| | | ACCAAGGCGACGATCCGTAGCCGACCTGAGAGGGTGATCGGCCACACTGGA |
| | | ACTGAGACACGGTCCAGACTCCTACGGGAGGCAGCAGTGGGGAATATTGCAC |
| | | AATGGGCGAAAGCCTGATGCAGCAACGCCGCGTGAGCGATGAAGGCTTTCGA |
| | | GTCGTAAAGCTCTGTCGCAGGGGAAGAAAAAGACGGTACCCTGTGAGAAAG |
| | | CCCCGGCTAACTACGTGCCAGCAGCCGCGGTAATACGTAGGGGGCAAGCGTT |
| | | ATCCGGAATTACTGGGCGTAAAGGGTGCGTAGGCGGCTTTTCAAGTCAAGGC |
| | | TTAAAGGCTAACGGCTCAACCGTAGTAAGGCCTTGAAACTGGAGAGCTTGAG |
| | | TATCGGAGAGGAAAGTGGAATTCCCAGTGTAGCGGTAAAATGCGTAGATATT |
| | | GGGAGGAACACCAGTGGCGAAGGCGACTTTCTGGACGATGACTGACGCTGA |
| | | GGCACGAAAGCGTGGGGAGCAAACAGGATTAGATACCCTGGTAGTCCACGC |
| | | CGTAAACGATGAGTACTAGGTGTCAGGTGTCAAAGCTTGGTGCCGCAGTTAA |
| | | CACAGTAAGTACTCCGCCTGGGGAGTACGCTCGCAAGAGTGAAACTCAAAGG |
| | | AATTGACGGGGACCCGCACAAGTAGCGGAGCATGTGGTTTAATTCGAAGCAA |
| | | CGCGAAGAACCTTACCTAAGCTTGGCATCCCGTTGACCGGGGCTTAATCGCCC |
| | | CTTTCCCTTCGGGGACAGCGGAGACAGGTGGTGCATGGTTGTCGTCAGCTCGT |
| | | GTCGTGAGATGTTGGGTTAAGTCCCGCAACGAGCGCAACCCTTGTTTTTAGTT |
| | | GCCAGCGGTTTGGCCGGGCACTCTAAAAAGACTGCCGAGGATAACTCGGAGG |
| | | AAGGTGGGGATGACGTCAAATCATCATGCCCCTTATGCTTAGGGCTACACACG |
| | | TGCTACAATGGCTGTTACAAAGGGCCGCGAGGCCGCAAGGCGGAGCCAATC |
| | | CCATAAAAACAGTCTCAGTTCGGATTGCAGGCTGAAACTCGCCTGCATGAAGT |
| | | CGGAGTTACTAGTAATCGCGAATCAGCATGTCGCGGTGAATGCGTTCCCGGGT |
| | | CTTGTACACACCGCCCGTCACACCATGGAAGCTTGGGGCACCCAAAGTCCGT |
| | | GATCCAACGAAAGAGGAAGCGGCCTAAGGTGAAACAAGTGACTGGGGTGAA |
| | | GTCGTAACAAGGTAGCCGTATCGGAAGGTGCGGCTGGATCACCTCCTT |
| Peptostrepto- | COT-006 | >006OB016 \| Peptostreptococcaceae XI [G-1] sp. \| COT-006 \| Clone OB016 |
| coccaceae XI | | \| JN713159 \| 22 \| |
| [G-1] sp. | | GATGAACGCTGGCGGCGTGCTTAACACATGCAGGTCGAACGAAGCAGGAAG |
| COT-006 | | GAAGAAGTTTTTCGAAGCCGATTTTTTCCTTGCTTAGTGGCGGACGGGTGCGT |
| (SEQ ID NO.: 27) | | AACACGTAGGTAACCTGCCCTGTTCATTGGAATAACTTCGGGAAACCGGTGCT |
| | | AATGCCAAATAACATCGCCTGTTGGCATCAAGAGGCGATCAAAGAATATCGG |
| | | AACAGGATGGGCCTGCGCCGGATTAGCTAGTTGGTGAGGTAATAGCTCACCA |
| | | AGGCAACGATCCGTAGCCGACCTGAGAGGGCGATCGGCCACACTGGAACTG |
| | | AGACACGGTCCAGACTCCTACGGGAGGCAGCAGTGGGGAATATTGCACAATG |
| | | GGCGAAAGCCTGATGCAGCAACGCCGCGTGAGCGAAGAAGGCTTTCGAGTC |
| | | GTAAAGCTCTGTCCTATGAGAAGATGATGACGGTACCATAGGAGGAAGCCCC |
| | | GGCTAACTACGTGCCAGCAGCCGCGGTAATACGTAGGGGGCAAGCGTTATCC |
| | | GGAATTACTGGGCGTAAAGGGTGCGTAGGCGGCTGTTCAAGTCAACGCTGAA |
| | | AGGCTAACGGCTCAACCGTAGTAAGGCGTTGAAACTGGAGAGCTTGAGTATC |
| | | GGAGAGGAAAGTAGAATTCCCAGTGTAGCGGTGAAATGCGTAGATATTGGGA |
| | | GGAATACCGGTGGCGAAGGCGACTTTCTGGACGAAAACTGACGCTGAGGCAC |
| | | GAAAGCGTGGGGAGCAAACAGGATTAGATACCCTGGTAGTCCACGCCGTAAG |
| | | CGATGAGTACTAGGTGTCAGGTGTCAAAACCTGGTGCCGCAGTTAACACAGT |
| | | AAGTACTCCGCCTGGGGAGTACGCTCGCAAGAGTGAAACTCAAAGGAATTGA |
| | | CGGGGACCCGCACAAGTAGCGGAGCATGTGGTTTAATTCGAAGCAACGCGAA |
| | | GAACCTTACCTAAGCTTGACATCCCGTTGACCGGGGTTTAATCGCCCCTTTCCC |
| | | CTCGGGGACAGCGGAGACAGGTGGTGCATGGTTGTCGTCAGCTCGTGTCGTG |
| | | AGATGTTGGGTTAAGTCCTGCAACGAGCGCAACCCCTATTTTTAGTTGCCAGC |
| | | GGTATGGCCGGGCACTCTAAAAAGACTGCCGGGGATAACCCGGAGGAAGGT |
| | | GGGGATGACGTCAAATCATCATGCCCCTTATGCCTAGGGCTACACACGTGCTA |
| | | CAATGGCTGTTACAAAGGGCTGCGACGCCGCAAGGCGAAGCCAATCCCATAA |
| | | AAACAGTCTCAGTTCGGATTGCAGGCTGAAACTCGCCTGCATGAAGTCGGAG |
| | | TTACTAGTAATCGCAAATCAGCATGTTGCGGTGAATGCGTTCCCGGGTCTTGT |
| | | ACACACCGCCCGTCACACCATGGAAGCTTGGGGCACCCAAAGTCCGTCGTCT |
| | | AACTTCGGAAGAAGCGGCCTAAGGTGAAACAAGTGACTGGGGTGAAGTCGTA |
| | | ACAAGGTAGCCGTATCGGAAGGTGCGGCTGGATCACCTCCTT |
| Peptostrepto- | COT-104 | >104OH065 \| Peptostreptococcaceae XI [G-3] sp. \| COT-104 \| Clone OH065 |
| coccaceae XI | | \| JN713268 \| 30 \| |
| [G-3] | | GATGAACGCTGGCGGCGTGCCTAACACATGCAAGTCGAGCGAGAATCTTGGA |
| sp. COT-104 | | AGTGGAGACTTCGGTCGAAGCGACTAAGAGGAAAGCGGCGGACGGGTGAGT |
| (SEQ ID NO.: 28) | | AACGCGTGGGCAACCTGCCTCTTACTAGAGGATAGCCACCGGAAACGGTGAT |
| | | TAATACTGTATGAAGTGCATTTATCGCATGATAGATGTCCCAAAGATTTATCGG |
| | | TAAGAGATGGGCCCGCGTCTGATTAGCTAGTTGGCAAGGTAACGGCTTACCA |
| | | AGGCGACGATCAGTAGCCGACCTGAGAGGGTGAACGGCCACACTGGAACTG |
| | | AGACACGGTCCAGACTCCTACGGGAGGCAGCAGTGGGGAATATTCGGCAATG |
| | | GGGGGAACCCTGACCGAGCAACGCCGCGTGAGTGATTGAAGGTCTTCGGACT |
| | | GTAAAACTCTGTTGAATGGGAAGATAATGACGGTACCATTTGAGGAAGCCCC |
| | | GGCTAACTACGTGCCAGCAGCCGCGGTAATACGTAGGGGGCGAGCGTTATCC |
| | | GGAATCACTGGGCGTAAAGGGTGCGTAGGCGGGACATCAAGCCAGGGGTGA |
| | | AAGGCAGTGGCTCAACCATTGTAAGCCTTTGGAACTGATGTTCTTGAGTGCAG |
| | | GAGAGGTAAGTGGAATTCCTAGTGTAGAGGTAAAATTCGTAGATATTAGGAG |
| | | GAACACCGGTGGCGAAGGCGACTTACTGGACTGTAACTGACGCTGATGCACG |
| | | AAGGCGTGGGTAGCAAACAGGATTAGATACCCTGGTAGTCCACGCGGTAAAC |
| | | GATGAATACTAGGTATTGGGAATCAAATCTCGGTGCCGCAGCTAACGCATTAA |
| | | GTATTCCGCCTGGGGAGTACGCACGCAAGTGTGAAACTCAAAGGAATTGACG |
| | | GGGACCCGCACAAGTAGCGGAGCATGTGGTTTAATTCGTGGCAACGCGTAGA |
| | | ACCTTACCAGATCTTGACATCCCAATGAATATACTTTAATCGGTATAGCTCTTC |
| | | GGAGCATTGGAGACAGGTGGTGCATGGTTGTCGTCAGCTCGTGTCGTGAGAT |
| | | GTTGGGTTAAGTCCCGCAACGAGCGCAACCCTTGTGTTTAGTTGTTACCATTAA |
| | | GTTGAACCCTCTAAACAGACTGCCGTGGATAACGCGGAGGAAGGTGGGGATG |
| | | AGGTCAAATCATCATGCCCCTTACGATCTGGGCTACACACGTGCTACAATGGC |
| | | CGGTACAACGGGCAGCGAAGGAGCGATCCGGAGCAAAACCTAAAAAGCCGG |
| | | TCCCAGTTCGGATTGCAGGCTGCAACTCGCCTGCATGAAGTCGGAGTTACTAG |
| | | TAATCGCGGATCAGAATGTCGCGGTGAATGCGTTCTCGGGTCTTGTACACACC |
| | | GCCCGTCACACCATGGAAGTTCGGGGCGCCCGAAGCCGGTGGCCTAACCTTA |
| | | GGGAAGGAGCTGTCGAAGGTGAAACGAATGACTGGGGTGAAGTCGTAACAA |
| | | GGTAGCCGTATCGGAAGGTGCGGCTGGATCACCTCCTT |
| Peptostrepto- | COT-019 | >019OG085 \| Peptostreptococcaceae XI [G-4] sp. \| COT-019 \| Clone OG085 |
| coccaceae XI | | \| JN713182 \| 66 \| |
| [G-4] sp. | | GATGAACGCTGGCGGCGTGCCTAATACATGCAAGTCGAGCGAGAACTTATTA |
| COT-019 | | AATGGAGACTTCGGTCAAAGTGAGATAAGGGAAAGCGGCGGACGGGTGAGT |
| (SEQ ID NO.: 29) | | AATGCGTGGGAAACCTGCCCTTTAGAGTGGGATAGCAGTTGGAAACGACTAT |
| | | TAATACCCCATACTCTTGAGTTTTCGCATGAAGATTCAAGGAAAGATTTTATCG |
| | | CTAAAGGATGGTCCCACGTCTGATTAGCTAGTTGGTGAGGTAACGGCTCACCA |
| | | AGGCGACGATCAGTAGCCGACCTGAGAGGGTGATCGGCCACACTGGAACTG |
| | | AGACACGGTCCAGACTCCTACGGGAGGCAGCAGTAGGGAATATTGGGCAAT |
| | | GGGGGCAACCCTGACCCAGCAACGCCGCGTGAATGATGAAGGTCTTCGGATC |
| | | GTAAAATTCTTTTCTTGGGGAAGAACAAGGTGACGGTACCCAAGGAATAAGC |
| | | ACCGGCTAACCACGTGCCAGCAGCCGCGGTAATACGTAGGGTGCAAGCGTTA |
| | | TCCGGAATCACTGGGCGTAAAGGGTGCGTAGGCGGCCTATCAAGTCAGAAGT |
| | | TAAAGACAATGGCTTAACCATTGAAAGCTTTTGAAACTGATTGGCTTGAATAC |
| | | AGGAGAGGCAAGTGGAATTCCTAGTGTAGCGGTGAAATGCGTAGATATTAGG |
| | | AGGAACACCGGTGGCGAAGGCGACTTGCTGGACTGAGATTGACGCTGAGGC |
| | | ACGAAAGCGGGGGGAGCGAACAGGATTAGATACCCTGGTAGTCCACGCCGT |
| | | AAACGATGAATACTAGGTGTCGGCTAGAAATAGTCGGTGCCGGCGCAAACGC |
| | | AATAAGTATTCCGCCTGGGGAGTACGCACGCAAGTGTAAAACTCAAAGGAAT |
| | | TGACGGGGACCCGCACAAGCAGCGGAGCATGTAGTTTAATTCGACGCAACGC |
| | | GAAAACCTTACCAAGTCTTGACATCTGGATGACCGCACTTTAACAGGTGCCTC |
| | | TCTTCGGAGCATCCAAGACAGGTGGTGCATGGTTGTCGTCAGCTCGTGTCGTG |
| | | AGATGTTGGGTTAAGTCCCGCAACGAGCGCAACCCTTATTTTTAGTTGCCATC |
| | | AGGTGAAGCTGGGCACTCTAAAGAGACTGCCGTGGACAACACGGAGGAAGG |
| | | TGGGGATGACGTCAAATCATCATGCCCCTTATGATTTGGGCTACAAACGTGCT |
| | | ACAATGGCCGGTACAACGGGCAGCGAAGAGGTGACTCGGAGCGAATCCTAA |
| | | AAAGCCGGTCCCAGTTCGGATTGTAGGCTGCAACTCGCCTACATGAAGTTGG |
| | | AGTTGCTAGTAATCGTGAATCAGCATGTCGCGGTGAATGCGTTCCCGGGTCTT |
| | | GTACACACCGCCCGTCAAACCATGGAAGCTGGGGGTACCCGAAGTCAGTGAT |
| | | CTAACCCGCAAGGGAGGAAGCTGCCGAAGGTAAAACCAATGACTGGGGTTA |
| | | AGTCGTAACAAGGTAGCCGTATCGGAAGGTGCGGCTGGATCACCTCCTT |
| Peptostrepto- | COT-068 | >068OF007 \| Peptostreptococcaceae XI [G-11] sp. \| COT-068 \| Clone OF007 |
| coccaceae XI | | \| JN713233 \| 8 \| |
| [G-6] | | GATGAACGCTGGCGGCGTGCCTAACACATGCAAGTCGAGCGAGAACTTTGAG |
| sp. COT-068 | | AAAGAAGCTTCGGTAGATTTTTCAAAGGGAAAGCGGCGGACGGGTGAGTAAC |
| (SEQ ID NO.: 30) | | GCGTAGGCAACCTGCCCCATACAAAGGGATAGCAACGGGAAACTATTATTAA |
| | | AACCTTATGAAGTAAAGATGTTGCATGACATTTTTACCAAAGATTTATCGGTAT |
| | | GGGATGGGCCTGCGTCTGATTAGTTAGTTGGTGAGGTAACGGCTCACCAAGG |
| | | CGACGATCAGTAGCCGACCTGAGAGGGTGATCGGCCACATTGGAACTGAGAC |
| | | ACGGTCCAAACTCCTACGGGAGGCAGCAGTGGGGAATATTGCACAATGGGCG |
| | | AAAGCCTGATGCAGCAACGCCGCGTGAACGAAGAAGGCCCTTGGGTCGTAA |
| | | AGTTCTGTCCAAAGGGAAGATAATGACGGTACCTTTGGAGGAAGCCCCGGCT |
| | | AACTACGTGCCAGCAGCCGCGGTAATACGTAGGGGGCAAGCGTTATCCGGAA |
| | | TTATTGGGCGTAAAGGGTACGTAGGTGGTCTTTTAAGCGCAGGGTTTAAGGCA |
| | | ATGGCTTAACCATTGTATGCCTTGCGAACTGGAGGACTTGAGTGCAGGAGGG |
| | | GAAAGCGGAATTCCTAGTGTAGCGGTGAAATGCGTAGATATTAGGAGGAACA |
| | | CCAGCGGCGAAGGCGGCTTTCTGGACTGTAACTGACACTGAGGTACGAAAGC |
| | | GTGGGGAGCAAACAGGATTAGATACCCTGGTAGTCCACGCCGTAAACGATGA |
| | | GCACTAGATGTCGGGTAGAAATATTCGGTATCGCAGTTAACGCAATAAGTGCT |
| | | CCGCCTGGGGAGTACGCACGCAAGTGTGAAACTCAAAGGAATTGACGGGGA |
| | | CCCGCACAAGCAGCGGAGCATGTGGTTTAATTCGAAGCAACGCGAAGAACCT |
| | | TACCAGGACTTGACATCCCTCTGACAGGAGTGTAATGACTCTTTTCTACGGAC |
| | | AGAGGAGACAGGTGGTGCATGGTTGTCGTCAGCTCGTGTCGTGAGATGTTGG |
| | | GTTAAGTCCCGCAACGAGCGCAACCCTTGTCTTTAGTTGCCATCATTAAGTTG |
| | | GGCACTCTAAAGAGACTGCCGGGGACAACTCGGAGGAAGGTGGGGATGACG |
| | | TCAAATCATCATGCCCCTTATGTTCTGGGCTACACACGTGCTACAATGGCCGG |
| | | AACAAAGAGAAGCGAAATCGTGAGGTGGAGCGAATCTCAAAAACCGGCCCC |
| | | AGTTCGGATTGCAGGCTGCAACTCGCCTGCATGAAGTCGGAGTTGCTAGTAAT |
| | | CGCAGATCAGAATGCTGCGGTGAATGCGTTCCCGGGTCTTGTACACACCGCC |
| | | CGTCACACCATGGAAGTTGGGGGCGCCCAAAGTTGGTCGGCAGATAGACTAC |
| | | CTAAGGTGAAACCAATGACTGGGGTGAAGTCGTAACAAGGTAGCCGTATCGG |
| | | AAGGTGCGGCTGGATCACCTCCTT |
| Peptostrepto- | COT-030 | >030OB047 \| Peptostreptococcaceae XIII [G-1] sp. \| COT-030 \| Clone OB047 |
| coccaceae XIII | | \| JN713194 \| 25 \| |
| [G-1] | | GATTAACGCTGGCGGCGTGCGTAACACATGCAAGTCGAACGGTATTTTGAAA |
| sp. COT-030 | | GGAAGAGACTTCGGTCTTGGAAAGTCAAGAGAGAGTGGCGAACGGGTGAGT |
| (SEQ ID NO.: 31) | | AACGCGTGAGCAACCTGTCTTCCACAAAGGGATAGCTGCCGGAAACGGTAAT |
| | | TAATACCATATGAAGTTAACGAATCGCATGATAAGGTAACTAAAGAGAAATCG |
| | | GTGGAAGATGGGCTCGCGTCCGATTAGCTGGTTGGAGAGATAACAGCTCACC |
| | | AAGGCAATGATCGGTAGCCGGCTTGAGAGAGTGAACGGCCACACTGGAACT |
| | | GAGAGACGGTCCAGACTCCTACGGGAGGCAGCAGTGGGGAATTTTGCACAAT |
| | | GGGGGGAACCCTGATGCAGCGACGCCGCGTGAGCGAAGAAGGAATTCGTTT |
| | | CGTAAAGCTCTGTCCTATGGAAAGATAATGACGGTACCATAGGAGGAAGCCC |
| | | CGGCTAACTACGTGCCAGCAGCCGCGGTAATACGTAGGGGGCGAGCGTTGTC |
| | | CGGAATTATTGGGCGTAAAGGGTACGCAGGCGGTCTAAGAAGTCAGATGTTA |
| | | AAGCGTAGGGCTTAACCCTATAAAGCATATGAAACTGATAGACTTGAGTGAA |
| | | GGAGAGGAAAGTGGAATTCCTGGTGTAGCGGTGAAATGCGTAGATATCAGGA |
| | | GGAATACCGGTGGCGAAGGCGACTTTCTGGACTTTAACTGACGCTGAGGTAC |
| | | GAAAGCGCGGGGAGCAAACAGGATTAGATACCCTGGTAGTCCGCGCCGTAA |
| | | ACGATGAATGCTAGGTATTGGGAGTCAAATTTCAGTGCCGAAGTAAACACATT |
| | | ATGCATTCCGCCTGGGGAGTACGGTGGCAACACTGAAACTCAAAGGAATTGA |
| | | CGGGGACCCGCACAAGCAGCGGAGCATGTGGTTTAATTCGATGCAACGCGAA |
| | | GAACCTTACCGGGGCTTGACATATACTTGAAGTATGTAGAGATACATATGATT |
| | | CTTCGGAAACAGGTAAACAGGTGGTGCATGGTTGTCGTCAGCTCGTGTCGTGA |
| | | GATGTTGGGTTAAGTCCCGCAACGAGCGCAACCCCTATGCTTAGTTACCAGCA |
| | | GTAAGATGGGGACTCTAAGCAGACTGCCGATGATAAATCGGAGGAAGGCGG |
| | | GGAAGACGTCAAATCATCATGCCCTTTATGTCCCGGGCTACACACGTGCTACA |
| | | ATGGTCGGTACAGAGGGATGCGACATAGAGATATGAAGCGAAACCCAAAAA |
| | | GCCGGTCCCAGTTCGGATTGTAGGCTGCAATTCGCCTACATGAAGTCGGAGTT |
| | | GCTAGTAATCGCAAATCAGAATGTTGCGGTGAATGCGTTCCCGGGTCTTGTAC |
| | | ACACCGCCCGTCACACCATGGGAGTTAAAAATGCCCGAAGCCGGTGAGCTAA |
| | | CCGCAAGGGAGCAGCCGTCGAAGGTAGGGTTAATGACTGGGGTGAAGTCGT |
| | | AACAAGGTAGCCGTATCGGAAGGTGCGGCTGGATCACCTCCTT |
| Peptostrepto- | COT-077 | >077OC007 \| Peptostreptococcaceae XIII [G-2] sp. \| COT-077 \| Clone OC007 |
| coccaceae XIII | | \| JN713240 \| 67 \| |
| [G-2] | | GATGAACGTTGGCGACGTGCCTAACACATGCAAGTCGAGCGGTGAAAAGCG |
| sp. COT-077 | | GACTTAATCTTCGGAAAGAGGAAACTTGGAACAGCGGCGGACGGGTGAGTAA |
| (SEQ ID NO.: 32) | | TACATGAGCAACCTGCCTTGGACAGAGGGATAGCCGTGGGAAACCGCGATTA |
| | | ATACCTCATAAGACCCCACTATCGCATGATAGAGAGGTTAAAGATTCATCGGT |
| | | CCAAGATGGGCTCATGACCCATTAGCTAGTTGGTGAGATAACAGCCCACCAA |
| | | GGCGACGATGGGTAGCCGGCTTGAGAGAGTGACCGGCCACATTGGAACTGA |
| | | GACACGGTCCAGACTCCTACGGGAGGCAGCAGTGGGGAATATTGCACAATGG |
| | | JAGGAAACTCTGATGCAGCGACGTCGCGTGAGCGAAGAAGGTTTTCGAATCGT |
| | | AAAGCTCTGTCCTATGGGAAGAAGAAAGACGGTACCATAGAAGAAAGCCCCG |
| | | GCTAACTACGTGCCAGCAGCCGCGGTAATACGTAGGGGGCGAACGTTATCCG |
| | | GAATGATTGGGCGTAAAGGGTGCGTAGGCGGACCTGTAAGTCAGGTGTGAAA |
| | | GGCATGGGCTCAACCCATGTAAGCACTTGAAACTGCGGGCCTTGAGGGAAGG |
| | | AGAGGTAAGTGGAATTCCTAGTGTAGCGGTGAAATGCGTAGATATTAGGAGG |
| | | AATACCGGTGGCGAAGGCGACTTACTGGACTTTTACTGACGCTGAGGTACGA |
| | | AAGCGTGGGGAGCAAACAGGATTAGATACCCTGGTAGTCCACGCCGTAAATG |
| | | GTGAGTGCTAGGTGTCGGGAGTCAATCTCGGTGCCGACGTTAACACATTAAG |
| | | CACTCCGCCTGGGGAGTACGCACGCAAGTGTGAAACTCAAAGGAATTGACGG |
| | | GGACCCGCACAAGCAGCGGAGCATGTGGTTTAATTCGATGCAACGCGAAAAA |
| | | CCTTACCAGGACTTGACATATGGATGCCCGGTATAGAGATATACCCTTTCTTCG |
| | | GAACATTCATACAGGTGGTGCATGGTTGTCGTCAGCTCGTGTCGTGAGATGTT |
| | | GGGTTAAGTCCCGCAACGAGCGCAACCCCTATTCTTAGTTACTAACAGGTCAA |
| | | GCTGAGGACTCTAAGGAGACTGCCGGTGACAAACCGGAGGAAGATGGGGAT |
| | | GACGTCAAATCATCATGCCCTTTATGTCCTGGGCTACACACGTGCTACAATGG |
| | | TCGGTACAGAAGGAAGCGAGATAGCGATATCGAGCGAACCCCCAAAGCCGA |
| | | TCTCAGTTCGGATTGTTCTCTGCAACTCGAGAACATGAAGCCGGAGTTGCTAG |
| | | TAATTGCGAATCAGAATGTCGCAGTGAATGCGTTCCCGGGTCTTGTACACACC |
| | | GCCCGTCACACCATGGGAGTTGGTAATACCCGAAGCCTGTGAGCCAACCTGC |
| | | AAAGGAAGCAGCAGTCGAAGGTAGGATCAATGACTGGGGTGAAGTCGTAAC |
| | | AAGGTAGCCGTATCGGAAGGTGCGGCTGGATCACCTCCTT |
| Peptostreptococcus | COT-033 | >033OD020 \| Peptostreptococcus sp. \| COT-033 \| Clone OD020 \| JN713198 |
| sp. COT-033 | | \| 101 \| |
| (SEQ ID NO.: 33) | | GATGAACGCTGGCGGCGTGCCTAACACATGCAAGTCGAGCGCGGTTGTGCTT |
| | | AGTATTGAGTGTTTTATTGATATAAAACATTGAATTCTATGCACAACTGAGCGG |
| | | CGGACGGGTGAGTAACGCGTGGGTAACCTGCCCTATACACATGGATAACATA |
| | | CTGAAAAGTTTACTAATACATGATAAAATAGTTTTTCGGCATCGAAGAATTATC |
| | | AAAGTGTTTGCGGTATAGGATGGACCCGCGTCTGATTAGCTAGTTGGTGAGAT |
| | | AACTGCCCACCAAGGCGACGATCAGTAGCCGACCTGAGAGGGTGATCGGCC |
| | | ACATTGGAACTGAGACACGGTCCAAACTCCTACGGGAGGCAGCAGTGGGGA |
| | | ATATTGCACAATGGGCGCAAGCCTGATGCAGCAACGCCGCGTGAACGATGAA |
| | | GGTCTTCGGATCGTAAAGTTCTGTTGCAGGGGAAGATAATGACGGTACCCTGT |
| | | GAGGAAGCCCCGGCTAACTACGTGCCAGCAGCCGCGGTAATACGTAGGGGG |
| | | CTAGCGTTATCCGGATTTACTGGGCGTAAAGGGTGCGTAGGTGGTCTTTCAAG |
| | | TCGGTGGTTAAAGGCTACGGCTCAACCGTAGTAAGCCTCCGAAACGGTTAGA |
| | | CTTGAGTGCAGGAGAGGAAAGTGGAATTCCCAGTGTAGCGGTGAAATGCGTA |
| | | GATATTGGGAGGAACACCAGTAGCGAAGGCGGCTTTCTGGACTGCAACTGAC |
| | | ACTGAGGCACGAAAGCGTGGGTAGCAAACAGGATTAGATACCCTGGTAGTCC |
| | | ACGCCGTAAACGATGAGTACTAGGTGTCGGGGGTTACCCCCCTCGGTGCCGC |
| | | AGCTAACGCATTAAGTACTCCGCCTGGGGAGTACGCACGCAAGTGTGAAACT |
| | | CAAAGGAATTGACGGGGACCCGCACAGGTAGCGGAGCATGTGGTTTAATTCG |
| | | AAGCAACGCGAAGAACCTTACCTAAGCTTGACATCCCTCGGACCGGTGTTTAA |
| | | TCACACCTTTCCTTCGGGACTGAGGAGACAGGTGGTGCATGGTTGTCGTCAGC |
| | | TCGTGTCGTGAGATGTTGGGTTAAGTCCCGCAACGAGCGCAACCCTTGTCTTT |
| | | AGTTGCCATCATTAAGTTGGGCACTCTAGAGAGACTGCCAGGGACAACCTGG |
| | | AGGAAGGTGGGGATGACGTCAAATCATCATGCCCCTTATGCTTAGGGCTACA |
| | | CACGTGCTACAATGGGTGGTACAGAGGGTTGCCAAACCGTGAGGTGGAGCCA |
| | | ATCCCTTAAAGCCACTCTCAGTTCGGATTGTAGGCTGAAACTCGCCTACATGA |
| | | AGCTGGAGTTACTAGTAATCGCAGATCAGAATGCTGCGGTGAATGCGTTCCCG |
| | | GGTCTTGTACACACCGCCCGTCACACCATGGGAGTCGGAAGCACCCGAAGCC |
| | | GATTATCTAACCGCAAGGAGGAGATCGTCGAAGGTGGCGTCGATAACTGGGG |
| | | TGAAGTCGTAACAAGGTAGCCGTATCGGAAGGTGCGGCTGGATCACCTCCTT |
| Peptostreptococcus | COT-227 | >227PX064 \| Peptostreptococcus sp. \| COT-227 \| Clone PX064 \| JN713392 |
| sp. | | \| 4 \| |
| COT-227 | | GATGAACGCTGGCGGCGTGCCTAACACATGCAAGTCGAGCGCGACTGATTTG |
| (SEQ ID NO.: 34) | | ATGCTTGCATCGATGAAAGTTGAGCGGCGGACGGGTGAGTAACGCGTGGGCA |
| | | ACCTGCCCTGTACACATGGATAACATACTGAAAAGTTTACTAATACATGATAA |
| | | TATAGTTTTTCGGCATCGAAGAATTATCAAAGTGTTAGCGGTACAGGATGGGC |
| | | CCGCGTCTGATTAGCTAGTTGGTGAGATAACTGCCCACCAAGGCGACGATCA |
| | | GTAGCCGACCTGAGAGGGTGATCGGCCACATTGGAACTGAGACACGGTCCAA |
| | | ACTCCTACGGGAGGCAGCAGTGGGGAATATTGCACAATGGGCGCAAGCCTGA |
| | | TGCAGCAACGCCGCGTGAACGATGAAGGTCTTCGGATCGTAAAGTTCTGTTGC |
| | | AGGGGAAGACAATGACGGTACCCTGTGAGGAAGCCCCGGCTAACTACGTGC |
| | | CAGCAGCCGCGGTAATACGTAGGGGGCTAGCGTTATCCGGATTTACTGGGCG |
| | | TAAAGGGTGCGTAGGTGGTCCTTCAAGTCGGTGGTTAAAGGCTACGGCTCAA |
| | | CCGTAGTAAGCCTCCGAAACTGTTGGACTTGAGTGCAGGAGAGGAAAGTGGA |
| | | ATTCCCAGTGTAGCGGTGAAATGCGTAGATATTGGGAGGAACACCAGTAGCG |
| | | AAGGCGGCTTTCTGGACTGCAACTGACACTGAGGCACGAAAGCGTGGGTAGC |
| | | AAACAGGATTAGATACCCTGGTAGTCCACGCTGTAAACGATGAGTACTAGGT |
| | | GTCGGGGGTTACCCCCCTCGGTGCCGCAGCTAACGCATTAAGTACTCCGCCTG |
| | | GGGAGTACGCACGCAAGTGTGAAACTCAAAGGAATTGACGGGGACCCGCAC |
| | | AAGTAGCGGAGCATGTGGTTTAATTCGAAGCAACGCGAAGAACCTTACCTAA |
| | | GCTTGACATCCCTCGGACCGGTGTTTAATCACACCTTTCCTTCGGGACTGAGG |
| | | TGACAGGTGGTGCATGGTTGTCGTCAGCTCGTGTCGTGAGATGTTGGGTTAAG |
| | | TCCCGCAACGAGCGCAACCCTTGTCTTTAGTTGCCATCATTAAGTTGGGCACT |
| | | CTAGAGAGACTGCCAGGGATAACCTGGAGGAAGGTGGGGATGACGTCAAAT |
| | | CATCATGCCCCTTATGCTTAGGGCTACACACGTGCTACAATGGGTGGTACAGA |
| | | GGGTTGCCAAACCGTGAGGTGGAGCTAATCCCTTAAAGCCATTCTCAGTTCGG |
| | | ATTGTAGGCTGAAACTCGCCTACATGAAGCTGGAGTTACTAGTAATCGCAGAT |
| | | CGGAATGCTGCGGTGAATGCGTTCCCGGGTCTTGTACACACCGCCCGTCACA |
| | | CCATGGGAGTCGGAAACACCCGAAGCCGATTATCTAACCGCAAGGAGGAAGT |
| | | CGTCGAAGGTGGCGTCGATAACTGGGGTGAAGTCGTAACAAGGTAGCCGTAT |
| | | CGGAAGGTGCGGCTGGATCACCTCCTT |
| Porphyromonas | COT-109 | > 1092A134 \| Porphyromonas cangingivalis \| COT-109 \| Clone 2A134 \| |
| cangingivalis | | JN713276 \| 203 \| |
| COT-109 | | GATGAACGCTAGCGATAGGCTTAACACATGCAAGTCGAGGGGCAGCATAATT |
| (SEQ ID NO.: 35) | | TTAGCTTGCTAAGATTGATGGCGACCGGCGCACGGGTGCGTAACGCGTATGC |
| | | AACCTGCCTGTCAGTGGGGAATAACCCGGAGAAATCCGGACTAATACCGCAT |
| | | ACACTTTGGTTTCCGCATGGGGACTTTAGGAAAGATTTATTGCTGACAGATAG |
| | | GCATGCGTTCCATTAGATAGTTGGTGAGGTAACGGCTCACCAAGTCGACGATG |
| | | GATAGGGGAGCTGAGAGGCTTACCCCCCACACTGGAACTGAGACACGGTCCA |
| | | GACTCCTACGGGAGGCAGCAGTGAGGAATATTGGTCAATGGGCGAGAGCCT |
| | | GAACCAGCCAAGTCGCGTGAAGGAAGACGGTCCTATGGATTGTAAACTTCTTT |
| | | AGTAGGTGAGTACAAGCAGTTACGTGTAGCTGTAAGCAAGTAACCTAAGAAT |
| | | AAGTATCGGCTAACTCCGTGCCAGCAGCCGCGGTAATACGGAGGATACGAGC |
| | | GTTATCCGGATTTATTGGGTTTAAAGGGTGCGTAGGTGGCTTTATAAGTCAGT |
| | | GGTGAAAAGCTGTGGCTCAACCATAGTCTTGCCGTTGAAACTGTGGAGCTTGA |
| | | GTGCGGATGGGGTAGGCGGAACGCGTAGTGTAGCGGTGAAATGCATAGATAT |
| | | TACGCAGAACTCCGATCGCGAAGGCAGCTTACCAAACCGCAACTGACACTGA |
| | | AGCACGAAAGCGTGGGTATCAAACAGGATTAGATACCCTGGTAGTCCACGCA |
| | | GTAAACGATGATTACTCGGAGTATGCGATATAATGTATGCTCCCAAGGGAAAC |
| | | CGATAAGTAATCCACCTGGGGAGTACGCCGGCAACGGTGAAACTCAAAGGAA |
| | | TTGACGGGGGCCCGCACAAGCGGAGGAACATGTGGTTTAATTCGATGATACG |
| | | CGAGGAACCTTACCCGGGATTGAAATGTACATGACGGTTGGGCGAGAGCCTG |
| | | GCTTCCCTTCGGGGCATGTATGTAGGTGCTGCATGGTTGTCGTCAGCTCGTGC |
| | | CGTGAGGTGTCGGCTTAAGTGCCATAACGAGCGCAACCCACATCGTCAGTTA |
| | | CTAACAGGTAGAGCTGAGGACTCTGACGAGACTGCCGTCGTAAGGCGCGAG |
| | | GAAGGTGTGGATGACGTCAAATCAGCACGGCCCTTACATCCGGGGCGACACA |
| | | CGTGTTACAATGGTAGGGACAAAGGGCAGCTACCTGGCGACAGGATGCGAAT |
| | | CTCCAAACCCTATCTCAGTTCGGATCGGAGTCTGCAACTCGACTCCGTGAAGC |
| | | TGGATTCGCTAGTAATCGCGCATCAGCCATGGCGCGGTGAATACGTTCCCGG |
| | | GCCTTGTACACACCGCCCGTCAAGCCATGGAAGTCGGGGGTACCTGAAGTGC |
| | | GTCACCGCAAGGATCGCACGAGGGTAAGACCGGTAACTGGGGCTAAGTCGTA |
| | | ACAAGGT |
| Porphyromonas | COT-022 | >022OB003 \| Porphyromonas gingivicanis \| COT-022 \| Clone OB003 \| |
| gingivicanis COT- | | JN713184 \| 313 \| |
| 022 | | GATGAACGCTAGCGATAGGCTTAACACATGCAAGTCGAGGGGCAGCATAATT |
| (SEQ ID NO.: 36) | | TTAGCTTGCTAAGATTGATGGCGACCGGCGCACGGGTGCGTAACGCGTATGC |
| | | AACCTGCCTGTAATTAGGGGATAGCTCGGTGAAAGTCGAATTAATACCCTATA |
| | | TTCTATATTTTTCGCATGATTGATATAGGAAAGATTTATTGATTACAGATGGGC |
| | | ATGCGTTCCATTAGCTTGTTGGTAAGGTAACGGCTTACCAAGGCAACGATGGA |
| | | TAGGGGGACTGAGAGGTTGACCCCCCACACTGGTACTGAGACACGGACCAG |
| | | ACTCCTACGGGAGGCAGCAGTGAGGAATATTGGTCAATGGGCGCAAGCCTGA |
| | | ACCGGCCAAGTCGCGTGAAGGACGACGGTCCTATGGATTGTAAACTTCTTTTG |
| | | TAGAGGAATAATGATGGCCACGAGTGGCTGAAGGGAATGTACTCTACGAATA |
| | | AGTATCGGCTAACTCCGTGCCAGCAGCCGCGGTAATACGGAGGATACGAGCG |
| | | TTATCCGGATTTATTGGGTTTAAAGGGTGCGTAGGTGGCGAATTAAGTCAGTG |
| | | GTGAAAAGCTGCAGCTCAACTGTAGTCTTGCCGTTGAAACTGGTTTGCTTGAG |
| | | AGGAGACGAGGTATGCGGAATGTGTGGTGTAGCGGTGAAATGCATAGATATC |
| | | ACACAGAACGCCGATTGCGAAGGCAGCGTACCAGGCTCTTTCTGACACTGAA |
| | | GCACGAAAGCGTGGGTATCAAACAGGATTAGATACCCTGGTAGTCCACGCTG |
| | | TAAACGATGAATACTAGATTTTTGCGATATACAGTAAGAGTCTAAGCGAAAGC |
| | | GATAAGTATTCCACCTGGGGAGTACGCCGGCAACGGTGAAACTCAAAGGAAT |
| | | TGACGGGGGCCCGCACAAGCGGAGGAACATGTGGTTTAATTCGATGATACGC |
| | | GAGGAACCTTACCTGGGATTGAAATTTAGGAGAACGATTTATGAAAGTAGATT |
| | | TTCCCTTCGGGGCTCCTGAGCAGGTGCTGCATGGTTGTCGTCAGCTCGTGCCG |
| | | TGAGGTGTCGGCTTAAGTGCCATAACGAGCGCAACCCGCGTCGATAGTTACT |
| | | AACAGGTAATGCTGAGGACTCTATCGAGACAGCCGTCGTAAGACGAGAGGAA |
| | | GGGGCGGATGACGTCAAATCAGCACGGCCCTTACATCCAGGGCGACACACGT |
| | | GTTACAATGGCAAGGACAAAGGGAAGCCACATAGCGATATGGAGCAGATCCT |
| | | CAAACCTTGTCCCAGTTCGGATCGGAGTCTGCAACTCGACTCCGTGAAGCTGG |
| | | ATTCGCTAGTAATCGCGCATCAGCCATGGCGCGGTGAATACGTTCCCGGGCCT |
| | | TGTACACACCGCCCGTCAAGCCATGGGAGTCGGGGGTACCTGAAGAGTGTAA |
| | | CCGTAAGGAGCGCTTAAGGGTAATACCGGTGACTGGGGCTAAGTCGTAACAA |
| | | GGTAGCCGTACCGGAAGGTGTGGCTGGATCACCTCCTT |
| Porphyromonas | COT-052 | >052QC036 \| Porphyromonas gulae \| \| COT-052 \| Clone QC036 \| JN713221 |
| gulae \| COT-052 | | \| 184 \| |
| (SEQ ID NO.: 37) | | GATGAACGCTAGCGATAGGCTTAACACATGCAAGTCGAGGGGCAGCATGAAC |
| | | TTAGCTTGCTAAGTTTGATGGCGACCGGCGCACGGGTGCGTAACGCGTATGC |
| | | AACTTGCCTTACAGAGGGGGATAACCCGTTGAAAGACGGACTAATACCGCAT |
| | | ACACTTGCTTGGTTGCATGATCGGGCAAGGAAATATTTATAGCTGTAAGATAG |
| | | GCATGCGTCCCATTAGCTGGTTGGTGAGGTAACGGCTCACCAAGGCAACGAT |
| | | GGGTAGGGGAACTGAGAGGTTTATCCCCCACACTGGTACTGAGACACGGACC |
| | | AGACTCCTACGGGAGGCAGCAGTGAGGAATATTGGTCAATGGGCGAGAGCCT |
| | | GAACCAGCCAAGTCGCGTGAAGGATGACTGTCCTAAGGATTGTAAACTTCTTT |
| | | TATACGGGAATAACGGGCGATACGTGTATTGCTGTGAATGTACCGTAAGAATA |
| | | AGCATCGGCTAACTCCGTGCCAGCAGCCGCGGTAATACGGAGGATGCGAGC |
| | | GTTATCCGGATTTATTGGGTTTAAAGGGTGCGTAGGTTGTTCGGTAAGTCAGC |
| | | GGTGAAAACTGAGCGCTCAACGTTCAGCGTGCCGTTGAAACTGCCGGGCTTG |
| | | AGTTCAGTGGCGGCAGGCGGAATTCGTGGTGTAGCGGTGAAATGCATAGATA |
| | | TCACGAGGAACTCCGATTGCGAAGGCAGCTTGCCATACTGCGACTGACACTG |
| | | AAGCACGAAGGCGTGGGTATCAAACAGGATTAGATACCCTGGTAGTCCACGC |
| | | AGTAAACGATGATTACTAGGAGTTTGCGATATACCGTCAAGCTTCCACAGCGA |
| | | AAGCGTTAAGTAATCCACCTGGGGAGTACGCCGGCAACGGTGAAACTCAAAG |
| | | GAATTGACGGGGGCCCGCACAAGCGGAGGAACATGTGGTTTAATTCGATGAT |
| | | ACGCGAGGAACCTTACCCGGGATTGAAATGTAGACGACGGATGGTGAAAGCC |
| | | GTCTTCCCTTCGGGGCGTCTATGTAGGTGCTGCATGGTTGTCGTCAGCTCGTG |
| | | CCGTGAGGTGTCGGCTTAAGTGCCATAACGAGCGCAACCCACATCGGTAGTT |
| | | GCTAACAGGTTTAGCTGAGGACTCTACCGAGACTGCCGTCGTAAGGCGTGAG |
| | | GAAGGTGTGGATGACGTCAAATCAGCACGGCCCTTACATCCGGGGCTACACA |
| | | CGTGTTACAATGGGAGGGACAAAGGGCAGCTACCGGGCGACCGGATGCGAA |
| | | TCTCGAAACCCTTCCCCAGTTCGGATCGGAGTCTGCAACTCGACTCCGTGAAG |
| | | CTGGATTCGCTAGTAATCGCGCATCAGCCATGGCGCGGTGAATACGTTCCCG |
| | | GGCCTTGTACACACCGCCCGTCAAGCCATGGGAGTCGGGGGTACCTGAAGGG |
| | | CGTAACCGCAAGGGGCGCACTAGGGTAATACCGGTGACTGGGGCTAAGTCGT |
| | | AACAAGGTAGCCGTACCGGAAGGTGCGGCTGGAACACCTCCTT |
| Porphyromonas | COT-192 | >192QD016 \| Porphyromonas macacae \| COT-192 \| Clone QD016 \| |
| macacae COT- | | JN713359 \| 16 \| |
| 192 | | GATGAACGCTAGCGATAGGCTTAACACATGCAAGTCGAGGGGCAGCATGGTC |
| (SEQ ID NO.: 38) | | TTAGCTTGCTAAGACTGATGGCGACCGGCGCACGGGTGCGTAACGCGTATGT |
| | | AACTTGCCTGATAGAAAGGGATAACCCGGTGAAAGTCGGACTAATACCTTAT |
| | | GGTCTTGGGTTATTGCATGATGATTCAAGTAAAGATTAATTGCTATCAGATAGG |
| | | CATGCGTTCCATTAGTTAGTTGGTGAGGTAACGGCTCACCAAGGCGACGATG |
| | | GATAGGGGAACTGAGAGGTTTATCCCCCACACTGGTACTGAGACACGGACCA |
| | | GACTCCTACGGGAGGCAGCAGTGAGGAATATTGGTCAATGGGCGAGAGCCT |
| | | GAACCAGCCACGTCGCGTGAAGGAAGACGGTTCTATGGATTGTAAACTTCTTT |
| | | TATAGTGGATTAAAGTTATCCACGCGTGGATATTTGCAAGTACCCTATGAATA |
| | | AGCATCGGCTAACTCCGTGCCAGCAGCCGCGGTAATACGGAGGATGCGAGC |
| | | GTTATCCGGATTTATTGGGTTTAAAGGGTGCGTAGGTGGCCTGATAAGTCGGG |
| | | GGTGAAAAGCTGTGGCTCAACCACAGTCTTGCCTTCGAAACTGTTTGGCTTGA |
| | | GTATAGATGAAGTAGGCGGAATTTGTGGTGTAGCGGTGAAATGCATAGATAT |
| | | CACGAGGAACTCCGATTGCGAAGGCAGCTTACTAAGTTATGACTGACACTGA |
| | | AGCACGAAAGCGTGGGTATCAAACAGGATTAGATACCCTGGTAGTCCACGCA |
| | | GTAAACGATGATAACTGGGCGTATGCGATATACAGTATGCTCCTGAGCGAAA |
| | | GCGTTAAGTTATCCACCTGGGGAGTACGCCGGCAACGGTGAAACTCAAAGGA |
| | | ATTGACGGGGGCCCGCACAAGCGGAGGAACATGTGGTTTAATTCGATGATAC |
| | | GCGAGGAACCTTACCCGGGATTGAAATTTAGCGGACTATGTATGAAAGTACAT |
| | | ATCCTGTCACAAGGCCGCTAAGTAGGTGCTGCATGGTTGTCGTCAGCTCGTGC |
| | | CGTGAGGTGTCGGCTTAAGTGCCATAACGAGCGCAACCCACGTTGTCAGTTAC |
| | | TATCGGGTAAAGCCGAGGACTCTGACAAGACTGCCGTCGTAAGGCGCGAGGA |
| | | AGGTGTGGATGACGTCAAATCAGCACGGCCCTTACATCCGGGGCGACACACG |
| | | TGTTACAATGGTGGGGACAAAGGGCAGCTACTTGGTGACAAGATGCGAATCT |
| | | CCAAACCCCATCCCAGTTCGGATCGTAGTCTGCAACTCGACTATGTGAAGCTG |
| | | GATTCGCTAGTAATCGCGCATCAGCCATGGCGCGGTGAATACGTTCCCGGGC |
| | | CTTGTACACACCGCCCGTCAAGCCATGGGAGTCGGGAGTACCTAAAGCACGT |
| | | AACCGCGAGGGGCGTGTTAGGGTAATACCGGTGACTGGGGCTAAGTCGTAAC |
| | | AAGGT |
| Porphyromonas | COT-108 | >108QD022 \| Porphyromonas canoris \| COT-108 \| Clone QD022 \| JN713274 |
| sp. COT-108 | | \| 68 \| |
| (SEQ ID NO.: 39) | | GATGAACGCTAGCGATAGGCTTAACACATGCAAGTCGAGGGGCAGCATAGA |
| | | GTCAGCTTGCTGATTTAGATGGCGACCGGCGGATGGGTGCGTAACGCGTATG |
| | | CAACTTACCTGTCAGAGGGGGATAACCCGGAGAAATTCGAACTAATACCGCA |
| | | TATACTTGAGATGCTGCATGGTATTTCAAGGAAATATTTATAGCTGACAGATAG |
| | | GCATGCGATCCATTAGCTAGTAGGTGAGGTAACGGCTCACCTAGGCGACGAT |
| | | GGATAGGGGAACTGAGAGGTTAAACCCCCACACTGGTACTGAGACACGGAC |
| | | CAGACTCCTACGGGAGGCAGCAGTGAGGAATATTGGTCAATGGGCGAGAGC |
| | | CTGAACCAGCCAATTCGCGTGAAGGACGACTGCCCTATGGGTTGTAAACTTCT |
| | | TTAGTAGGGGATTAAAGTTTGCCTTGCGAGGCAATTTGCAAGTACCCTAAGAA |
| | | TAAGTATCGGCTAACTCCGTGCCAGCAGCCGCGGTAATACGGAGGATACGAG |
| | | CGTTATCCGGATTTATTGGGTTTAAAGGGTGCGTAGGTGGCTTTTTAAGTCAGT |
| | | GGTGAAAAGCTGTGGCTCAACCATAGTCTTGCCGTTGAAACTGAGGAGCTTG |
| | | AGTGTAGATGCTGTAGGCGGAACGCGTAGTGTAGCGGTGAAATGCATAGATA |
| | | TTACGCAGAACTCCGATTGCGAAGGCAGCTTACAAAGTTACAACTGACACTGA |
| | | AGCACGAGAGCGTGGGTATCAAACAGGATTAGATACCCTGGTAGTCCACGCC |
| | | GTAAACGATGATTACTAAGAGTATGCGATATAATGTATGTTCTACAGCGAAAG |
| | | CGTTAAGTAATCCACCTGGGGAGTACGTCGGCAACGATGAAACTCAAAGGAA |
| | | TTGACGGGGGCCCGCACAAGCGGAGGAACATGTGGTTTAATTCGATGATACG |
| | | CGAGGAACCTTACCCGGGATTGAAATGTGCATGACGTAAATTGGAGACAGTTT |
| | | ATACCCTTCGGGGCATGTATGTAGGTGCTGCATGGTTGTCGTCAGCTCGTGCC |
| | | GTGAGGTGTCGGCTTAAGTGCCATAACGAGCGCAACCCACATTGTCAGTTACT |
| | | AACAGGTAGAGCTGAGGACTCTGGCGAGACTGCCGGCGTAAGCCGTGAGGA |
| | | AGGTGTGGATGACGTCAAATCAGCACGGCCCTTACATCCGGGGCGACACACG |
| | | TGTTACAATGGTGAGGACAAAGGGCAGCTACCTGGCGACAGGGTGCGAATCT |
| | | CCAAACCTCATCTCAGTTCGGATCGGAGTCTGCAACTCGACTCTGTGAAGCTG |
| | | GATTCGCTAGTAATCGCGCATCAGCCATGGCGCGGTGAATACGTTCCCGGGC |
| | | CTTGTACACACCGCCCGTCAAGCCATGGAAGTCGGGAGTACCTGAAGTACGT |
| | | CACCGCGAGGATCGTACTAGGGTAATACCGGTAACTGGGGCTAAGTCGTAAC |
| | | AAGGT |
| Synergistales [F- | COT-138 | >138PY001 \| Synergistaceae [G-2] sp. \| COT-138 \| Clone PY001 \| JN713304 |
| 2, G-1] sp. COT- | | \| 74 \| |
| 138 | | GATGAACGCTGGCGGCGTGCCTAACACATGCAAGTCGAACGGGGTTATATTG |
| (SEQ ID NO.: 40) | | GTTAGTTTACTAAGCGATATGACTTAGTGGCGGACGGGTGAGTAACGCGTGA |
| | | GGACCTATCCCAAGCTGGGGGACAACAGCTGGAAACGGTTGCTAATACCGCA |
| | | TAAGCGCATAATCTGCGTAAAAGGAGAGATCCGGCATGGGAGGGGCTCGCGT |
| | | CCTATCAGCTAGTAGGTGGGGTAAAGGCCTACCTAGGCGATGACGGGTAGCC |
| | | GGCCTGAGAGGGCGCACGGCCACACTGGGACTGAGATACGGCCCAGACTCC |
| | | TACGGGAGGCAGCAGTGGGGAATATTGGGCAATGGGGGGAACCCTGACCCA |
| | | GCGACGCCGCGTGAGTGAAGAAGTCCTTCGGGACGTAAAGCTCTGTTGTGTG |
| | | GGAAGAAGAGGATGACGGTACCACACGAGGAAGCCCCGGCAAACTACGTGC |
| | | CAGCAGCCGCGGTAATACGTAGGGGGCGAGCGTTGTCCGGAATTACTGGGC |
| | | GTAAAGGGCACGCAGGCGGAATGGTAAGTCAGTTGTGAAAGGCTGTGGCTCA |
| | | ACCACAGGGAGACGACTGATACTGTCAGTCTAGAGTATGTGAGAGGGAAGTG |
| | | GAATTCCCGGTGTAGCGGTGAAATGCGTAGATATCGGGAGGAACACCAGTGG |
| | | CGAAGGCGGCTTCATGGCACATAACTGACGCTCATGTGCGAAAGCTAGGGTA |
| | | GCGAACGGAATTAGATACTCCGGTAGTCCTAGCCGTAAACGATGGATACTAG |
| | | GTGTGGGTGTCGCAGGGCATCCGTGCCGGAGTCAACGCGTTAAGTATCCCGC |
| | | CTGGGGACTACGGCCGCAAGGCTGAAACTCAAAGGAATTGACGGGGGCCCG |
| | | CACAAGCGGTGGAGCACGTGGTTTAATTCGATGCAAACCGAAGAACCTTACC |
| | | TGGGTTTGACATGTATATGTTAGAGAAGCGAGAGCGGATCGACCACAGTTTAC |
| | | TGTGGAGTATACACAGGTGCTGCATGGCTGTCGTCAGCTCGTGTCGTGAGATG |
| | | TTGGGTTAAGTCCCGCAACGAGCGCAACCCCTGTTGTCAGTTGCTAACGGTTA |
| | | GAGCCGAGCACTCTGGCGAGACTGCCGTCGACAAGACGGAGGAAGGTGGGG |
| | | ACGACGTCAAGTCATCATGGCCTTTATGTCCAGGGCGACACACGTGCTACAAT |
| | | GGTATGCACAGAGGGAAGCGAAGTGGTGACATGGAGCGGATCCAAGGAAGC |
| | | ATATCTCAGTTCGGATTGTAGTCTGCAACTCGGCTACATGAAGCCGGAATCGC |
| | | TAGTAATCGCAGATCAGCCAAGCTGCGGTGAATACGTTCCCGGGCCTTGTACA |
| | | CACCGCCCGTCACACCACTCGAGTTGTCTGCACCCGAAGCCAGTGGCATAAC |
| | | CGCAAGGGATGAGCTGTCTAAGGTGTGGGGGGTAAGGGGGGTGAAGTCGTA |
| | | ACAAGGT |
| Tannerella | COT-023 | >023OB071 \| Tannerella forsythus \| COT-023 \| Clone OB071 \| JN713185 |
| forsythus COT- | | \| 27 \| |
| 023 | | GATGAACGCTAGCGATAGGCTTAACACATGCAAGTCGAGGGGCAGCGAGGG |
| (SEQ ID NO.: 41) | | TAGCAATACCTGTCGGCGACCGGCGCACGGGTGAGTAACGCGTATGTAACCT |
| | | GCCCGCAACAGGGGGATAACCCGGCGAAAGTCGGACTAATACCGCATAAAA |
| | | CAGGGGTCCCGCATGGGAATATTTGTTAAAGATTTATCGGTTGCGGATGGGCA |
| | | TGCGTACCATTAGGTAGTTGGTGAGGTAACGGCTCACCAAGCCTTCGATGGTT |
| | | AGGGGTTCTGAGAGGAAGGTCCCCCACACTGGTACTGAGACACGGACCAGA |
| | | CTCCTACGGGAGGCAGCAGTGAGGAATATTGGTCAATGGGCGAGAGCCTGAA |
| | | CCAGCCAAGTCGCGTGAAGGATGACTGCCCTATGGGTTGTAAACTTCTTTTAC |
| | | AAGGGAATAAAATGAGATACGTGTATTTTATTGCATGTACCTTGGGAATAAGC |
| | | ATCGGCTAACTCCGTGCCAGCAGCCGCGGTAATACGGAGGATGCGAGCGTTA |
| | | TCCGGATTTATTGGGTTTAAAGGGTGCGTAGGTGGGCTGTTAAGTCAGCGGTG |
| | | AAAGTTTGTCGCTTAACGATAAAATTGCCGTTGAAACTGGTAGTCTTGAGTATA |
| | | GATGAAGTAGGCGGAATGCGTGGTGTAGCGGTGAAATGCATAGAGATCACGC |
| | | AGAACTCCGATTGCGAAGGCAGCTTACTAAGGTATAACTGACACTGAAGCAC |
| | | GAAAGCGTGGGTATCAAACAGGATTAGATACCCTGGTAGTCCACGCAGTAAA |
| | | CGATGATTACTAGGAGTTTGCGATATACAGTAAGCTCTACAGCGAAAGCGTTA |
| | | AGTAATCCACCTGGGGAGTACGCCGGCAACGGTGAAACTCAAAGGAATTGAC |
| | | GGGGGCCCGCACAAGCGGAGGAACATGTGGTTTAATTCGATGATACGCGAG |
| | | GAACCTTACCCGGGATTGAAATGTAGACGACGGACAGTGAGAGCTGTCTTCC |
| | | CTTCGGGGCGTCTATGTAGGTGCTGCATGGTTGTCGTCAGCTCGTGCCGTGAG |
| | | GTGTCGGCTTAAGTGCCATAACGAGCGCAACCCTGACTGTCAGTTGCTAACAG |
| | | GTTAAGCTGAGGACTCTGGCGGGACTGCCGGCGTAAGCTGTGAGGAAGGTTG |
| | | GGATGACGTCAAATCAGCACGGCCCTTACATCCGGGGCGACACACGTGTTAC |
| | | AATGGCAGGGACAAAGGGCAGCTACCGGGCGACCGGATGCCAATCTCCAAA |
| | | CCCTGTCTCAGTTCGGATCGGAGTCTGCAACTCGACTCCGTGAAGCTGGATTC |
| | | GCTAGTAATCGCGCATCAGCCATGGCGCGGTGAATACGTTCCCGGGCCTTGT |
| | | ACACACCGCCCGTCAAGCCATGGGAGCCGGGGGTACCTGAAGTCCGTAACC |
| | | GCGAGGATCGGCCTAGGGTAAAACTGGTGACTGGGGCTAAGTCGTAACAAG |
| | | GTAGCCGTACCGGAAGGTGCGGCTGGAACACCTCCTT |

The term “animal” as used in accordance with the present disclosure refers to a wide variety of animals, such as quadrupeds, primates, and other mammals. For example, the term “animal” can refer to domestic animals including, but not limited to, dogs, cats, horses, cows, ferrets, rabbits, pigs, rats, mice, gerbils, hamsters, goats, and the like. The term “animal” can also refer to wild animals including, but not limited to, wolf, bison, elk, deer, lion, tiger, and the like. In some embodiments, the animal is a companion animal. In certain instances, the animal is a dog or a cat.

The term “nucleic acid molecule” and “nucleotide sequence,” as used herein, refers to a single or double stranded covalently-linked sequence of nucleotides in which the 3′ and 5′ ends on each nucleotide are joined by phosphodiester bonds. The nucleic acid molecule can include deoxyribonucleotide bases or ribonucleotide bases, and can be manufactured synthetically in vitro or isolated from natural sources.

As used herein, the term “oral disease or disorder,” refers to a disease or disorder that occurs in an oral cavity of a subject (e.g., an animal) and that is caused by or is associated with one or more bacteria. For example, the disease or disorder can affect the teeth, structures that support the teeth such as periodontal ligament, alveolar bone, or the gums of the subject. Exemplary oral diseases or disorders of the present disclosure include, but are not limited to, periodontal disease, gingival stomatitis, odontoclastic resorptive lesions, and oral malodor.

As used herein, the term “periodontal disease,” also known as gum disease, refers to an inflammation or infection that affect the tissues surrounding the teeth. Periodontal disease can range in severity, e.g., from gingivitis (e.g., dental plaque-induced gingivitis) to periodontitis. An example of the range is found on https://avdc.org/avdc-nomenclature/.

The terms “isolated” or “purified”, used interchangeably herein, refers to a nucleic acid, a polypeptide, or other biological moiety that is removed from components with which it is naturally associated. The term “isolated” can refer to a polypeptide that is separate and discrete from the whole organism with which the molecule is found in nature or is present in the substantial absence of other biological macromolecules of the same type. The term “isolated” with respect to a polynucleotide can refer to a nucleic acid molecule devoid, in whole or part, of sequences normally associated with it in nature; or a sequence, as it exists in nature, but having heterologous sequences in association therewith; or a molecule disassociated from the chromosome.

As used herein, the term “biomarker” can refer to a characteristic that is objectively measured and evaluated as an indicator of physiological biological processes, pathogenic processes, or pharmacologic responses to a therapeutic intervention. In certain non-limiting embodiments, the term “biomarker” can refer to any substance, structure, or process that can be measured in the body or its products and influence or predict the incidence of outcome or disease.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. In certain embodiments, the decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of complications or symptoms. “Treatment” can also mean prolonging survival as compared to expected survival if not receiving treatment.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition (e.g., a dietary change, a functional food, a supplement, a nutraceutical composition, or a pharmaceutical composition) to change the composition of a microbiome having an unhealthy microbiome, the effective amount is an amount sufficient to bring the health status of the microbiome back to a healthy state, which is determined according to one of the methods disclosed herein. In certain embodiments, an effective treatment, as described herein, can also include administering a treatment in an amount sufficient to decrease any symptoms associated with an unhealthy microbiome. The decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of symptoms of an unhealthy microbiome. An effective amount can be administered in one or more administrations. A likelihood of an effective treatment described herein is a probability of a treatment being effective, i.e., sufficient to alter the microbiome, or treat or ameliorate a disorder and/or inflammation, as well as decrease the symptoms.

The word “substantially” does not exclude “completely” e.g. a composition which is “substantially free” from Y can be completely free from Y. Where necessary, the word “substantially” can be omitted from the definition of the present disclosure.

### 2. MICROORGANISMS IN THE ORAL MICROBIOME

The present disclosure relates to, inter alia, kits and related methods for detecting one or more microbial taxa (e.g., bacteria) in an oral microbiome of an animal. The one or more microbial taxa (e.g., bacteria) can be associated with an oral disease or disorder, e.g., periodontal disease, or with good oral health. The animal can be a companion animal, such as a dog or a cat.

In certain embodiments, the one or more microbial taxa are one or more bacteria. In some embodiments, the one or more bacteria associated with periodontal disease is selected from the group consisting of Actinomyces sp. COT-083, Actinomyces sp. COT-252, Anaerovorax sp. COT-125, Bacteroides sp. COT-040, Bergeyella zoohelcum COT-186, Capnocytophaga sp. COT-339, Clostridiales sp. COT-005, Clostridiales sp. COT-028, Desulfomicrobium orale COT-008, Filifactor sp. COT-064, Frigovirgula sp. COT-007, Fusobacterium sp. COT-169, Fusobacterium sp. COT-189, Gemella palaticanis COT-089, Granulicatella sp. COT-095, Helcococcus sp. COT-069, Lachnospiraceae XIVa [G-4] sp. COT-099, Lachnospiraceae XIVa [G-5] sp. COT-024, Leptotrichia sp. COT-345, Moraxella sp. COT-017, Moraxella sp. COT-018, Neisseria animaloris COT-016, Odoribacter denticanis COT-084, Pasteurellaceae sp. COT-271, Peptococcus sp. COT-044, Peptostreptococcaceae XI [G-1] sp. COT-004, Peptostreptococcaceae XI [G-1] sp. COT-006, Peptostreptococcaceae XI [G-3] sp. COT-104, Peptostreptococcaceae XI [G-4] sp. COT-019, Peptostreptococcaceae XI [G-6] sp. COT-068, Peptostreptococcaceae XIII [G-1] sp. COT-030, Peptostreptococcaceae XIII [G-2] sp. COT-077, Peptostreptococcus sp. COT-033, Peptostreptococcus sp. COT-227, Porphyromonas cangingivalis COT-109, Porphyromonas gingivicanis COT-022, Porphyromonas gulae I COT-052, Porphyromonas macacae COT-192, Porphyromonas sp. COT-108, Synergistales [F-2,G-1] sp. COT-138, or Tannerella forsythus COT-023.

Bacterial community profiles within an oral microbiome of an animal can vary depending on the source of a sample taken from the animal. For example, three discrete oral niches can include soft tissue surfaces, such as the lip, cheek, and tongue; hard tissue surfaces, such as the teeth; and saliva. In some embodiments, the oral niche is from a hard tissue surface, such as one or more teeth. In some embodiments, the oral niche includes the gingival margin, subgingival margin, or supragingival surface. The methods and kits of the disclosed subject matter can be used to detect bacteria in the oral microbiome of a wide variety of animals, such as quadrupeds, primates, and other mammals. The methods and kits of the disclosed subject matter are particularly well suited for use with companion animals, such as dogs, cats, and other domesticated animals.

### 3. COMPANION ANIMALS

The presently disclosed subject matter focuses on the health assessment of companion animals. In specific embodiments, the companion animal is a domestic dog.

### Dog Breeds

The present disclosure relates to, inter alia, methods for assessing health and well-being of animals. Characteristics of companion animals can vary, including by size, sex, breed, and species. However, for the most common member within this category, dogs, can in general provide an indication of the efficacy of a method when applied to other animals.

As used herein, the expression “size category” refers to the definition of the animal (e.g., dogs, cats, etc.) in terms of the average weight of the particular animal breed. Animals (e.g., dogs, cats, etc.) of the same breed can have relatively uniform physical characteristics, such as size, coat color, physiology, and behavior, as compared to animals of a different breed. It is noted that the discussion below is focused on dogs, however, other companion animals and wild animals are intended to be covered by the scope of this disclosure and the present disclosure is not intended to be limited to dogs.

The dog can be any breed of dog, including toy/extra-small, small, medium-small, medium, medium-large, large or extra-large/giant breeds. Non-limiting examples of toy/extra-small breeds include Affenpinscher, Australian Silky Terrier, Bichon Frise, Bolognese, Cavalier King Charles Spaniel, Chihuahua, Chinese Crested, Coton De Tulear, English Toy Terrier, Griffon Bruxellois, Havanese, Italian Greyhound, Japanese Chin, King Charles Spaniel Lowchen (Little Lion Dog), Maltese, Miniature Pinscher, Papillon, Pekingese, Pomeranian, Pug, Russian Toy, and Yorkshire Terrier. Examples of small breeds include, but are not limited to, French Bulldog, Beagle, Dachshund, Pembroke Welsh Corgi, Miniature Schnauzer, Cavalier King Charles Spaniel, Shih Tzu, and Boston Terrier. Examples of medium dog breeds include, but are not limited to, Bulldog, Cocker Spaniel, Shetland Sheepdog, Border Collie, Basset Hound, Siberian Husky, and Dalmatian. Examples of large breed dogs include, but are not limited to, Great Dane, Neapolitan mastiff, Scottish Deerhound, Dogue de Bordeaux, Newfoundland, English mastiff, Saint Bernard, Leonberger, and Irish Wolfhound. Other non-limiting examples of breeds include those listed in Wallis et al. (2021). Cross-breeds can generally be categorized as toy/extra-small, small, medium-small, medium, medium-large, large, and extra-large/giant dogs depending on their body weight. In certain embodiments, the dog is a toy/extra-small breed. In certain embodiments, the dog is a small, medium-small, medium, medium-large, large or extra-large/giant breed. In some embodiments, the dog is a mix of two or more breeds. In such instances, the mixed-breed dog can still be categorized by size depending on their body weight and can exhibit traits (e.g., behavioral traits, genetic traits, etc.) associated with each of the two or more breeds found in the dog.

The Fédération Cynologique Internationale currently recognizes 346 pure dog breeds. The breed of a dog can be identified, for example, either by observing its physical traits or by genetic analysis. A pedigree dog is the offspring of two dogs of the same breed, which is eligible for registration with a recognized club or society that maintain a register for dogs of that description. There are a number of pedigree dog registration schemes, of which the Kennel Club is the most well-known.

TABLE 2
A list of dog size categories.

| Size Category name | Weight range of dog breed | |
|---|---|---|
| Toy/Extra small | Up to 6.5 Kg | |
| Small | 6.5-9 | Kg |
| Medium-small | 9-15 | Kg |
| Medium-large | 15-30 | Kg |
| Large | 30-40 | Kg |

| | |
|---|---|
| Giant | Over 40 Kg |

In certain embodiments, the dog size categories are selected according to Salt et al., 2017 (Table 2). In other embodiments, the dog size categories are selected according to alternative designations. A small breed can correspond with animals that have an average body weight of from about 6.5 kilograms to about 9 kilograms. A medium breed can correspond with an animal that has an average body weight between about 9 kilograms and about 30 kilograms. A large breed can correspond with an animal that has an average body weight of between about 30 kilograms and about 40 kilograms. A giant breed can correspond with an animal that has an average body weight of between over about 40 kilograms.

### 4. METHODS

The disclosed methods of using the methods of the disclosed subject matter can include performing an assay on the sample to measure an amount of a microbial nucleic acid. In certain embodiments, the microbial nucleic acid can be a microbial DNA or RNA, e.g., a 16S ribosomal DNA (rDNA) or 16S ribosomal RNA (rRNA). Various assays for identifying the presence of bacteria or other markers associated with an oral disease or disorder (e.g., periodontal disease) or good oral health are known in the art. In an embodiment, the assay is quantitative polymerase chain reaction (qPCR).

For purposes of example, any of the disclosed methods can include performing an assay for testing for the presence and/or relative amounts of any bacteria disclosed herein. In certain embodiments, the bacteria are Peptostreptococcaeceae sp. and/or volatile organic compound producing bacteria, or any other periodontal bacterium, such as but not limited to Peptostreptococcus sp., Synergistes sp., Clostridiales sp., Eubacterium nodatum, Selenomonas sp., Bacteriodetes sp., Odoribacter denticanis, Desulfomicrobium ovale, Moraxella sp., Bacteroides denticanoris, Fillifactor villosus, Porphyromonas canoris, Porphyromonas gulae, Treponema denticola, or Porphyromonas salivosa.

Additionally or alternatively, any of the disclosed methods can include performing a universal qPCR assay which detects the presence of bacterial DNA in the dog oral microbiome. Universal primers are known to skilled people in the art and examples include those described in Ott et al., J. Clin. Microbiol. 2004 June; 42 (6): 2566-2572. Doi: 10.1128/JCM.42.6.2566-2572.2004, the contents of which is incorporated by reference in its entirety. Methods for performing qPCR assays are known in the art.

The methods can also include a step of extracting a nucleic acid, e.g., performing a DNA extraction, according to methods known in the art prior to performing the qPCR assay. Generally, a DNA extraction can be performed by lysing the cells containing the DNA and precipitating and purifying the DNA.

Additionally or alternatively, any of the disclosed methods can include detecting bacteria by testing the sample for the presence of bacteria. In certain embodiments, testing the sample can include for presence and/or relative abundance of one or more of the bacteria disclosed herein, e.g., bacteria associated with an oral disease or disorder (e.g., periodontal disease), bacteria associated with good oral health, or both. In certain embodiments, testing the sample can include detecting the abundance or increased relative abundance compared to a training data set (e.g., bacteria associated with good oral health, bacteria associated with an oral disease or disorder, bacteria not associated with good oral health or oral disease or disorder, and combinations thereof). Detecting a presence or relative increased abundance of one or more of the bacteria disclosed herein can, for instance, indicate that the animal has or is susceptible to developing an oral disease or disorder.

Additionally or alternatively, any of the disclosed methods can include detecting bacteria by testing the sample for an absence or relatively low abundance of bacteria. In certain embodiments, testing the sample can include for the absence or relatively low abundance of one or more of the bacteria disclosed herein, e.g., bacteria associated with an oral disease or disorder (e.g., periodontal disease). In certain embodiments, testing the sample can include detecting the decreased abundance or decreased relative abundance compared to a training data set. Detecting the absence or relatively low abundance of the one or more of the bacteria associated with the oral disease can, for instance, indicate that the animal does not have an oral disease or disorder or is less likely to develop an oral disease or disorder.

Additionally or alternatively, the disclosed methods of using the kits of the disclosed subject matter can include testing the sample for the presence and/or relative amounts of microbes associated with oral health. In an embodiment, the testing includes for the presence and/or relative amounts of a bacterial nucleic acid (e.g., DNA or RNA).

Additionally or alternatively, the disclosed methods can include determining oral health status of an animal. In a particular embodiment, the method for determining oral health status of an animal comprises quantifying one or more microbial taxa from a sample to determine abundance or relative abundance of the one or more microbial taxa, comparing the abundance or relative abundance of said one or more microbial taxa in the sample to abundance or relative abundance of the microbial taxa in a control data set, and determining the oral health status of the animal. In certain embodiments, the one or more microbial taxa are bacterial species. In certain embodiments, the one or more microbial taxa is selected from the group consisting of Actinomyces sp., Anaerovorax sp., Bacteroides sp., Bergeyella sp., Capnocytophaga sp., Clostridiales sp., Desulfomicrobium sp., Filifactor sp., Frigovirgula sp, Fusobacterium sp, Gemella sp., Granulicatella sp., Helcococcus sp., Lachnospiraceae sp., Leptotrichia sp., Moraxella sp., Neisseria sp., Odoribacter sp., Pasteurellaceae sp., Peptococcus sp., Peptostreptococcaceae sp., Peptostreptococcus sp. Porphyromonas sp., Synergistales sp., Tannerella sp., and combinations thereof. In an embodiment, the one or more microbial taxa is selected from the group consisting of Actinomyces sp. COT-083, Actinomyces sp. COT-252, Anaerovorax sp. COT-125, Bacteroides sp. COT-040, Bergeyella zoohelcum COT-186, Capnocytophaga sp. COT-339, Clostridiales sp. COT-005, Clostridiales sp. COT-028, Desulfomicrobium orale COT-008, Filifactor sp. COT-064, Frigovirgula sp. COT-007, Fusobacterium sp. COT-169, Fusobacterium sp. COT-189, Gemella palaticanis COT-089, Granulicatella sp. COT-095, Helcococcus sp. COT-069, Lachnospiraceae XIVa [G-4] sp. COT-099, Lachnospiraceae XIVa [G-5] sp. COT-024, Leptotrichia sp. COT-345, Moraxella sp. COT-017, Moraxella sp. COT-018, Neisseria animaloris COT-016, Odoribacter denticanis COT-084, Pasteurellaceae sp. COT-271, Peptococcus sp. COT-044, Peptostreptococcaceae XI [G-1] sp. COT-004, Peptostreptococcaceae XI [G-1] sp. COT-006, Peptostreptococcaceae XI [G-3] sp. COT-104, Peptostreptococcaceae XI [G-4] sp. COT-019, Peptostreptococcaceae XI [G-6] sp. COT-068, Peptostreptococcaceae XIII [G-1] sp. COT-030, Peptostreptococcaceae XIII [G-2] sp. COT-077, Peptostreptococcus sp. COT-033, Peptostreptococcus sp. COT-227, Porphyromonas cangingivalis COT-109, Porphyromonas gingivicanis COT-022, Porphyromonas gulae I COT-052, Porphyromonas macacae COT-192, Porphyromonas sp. COT-108, Synergistales [F-2,G-1] sp. COT-138, or Tannerella forsythus COT-023, and combinations thereof. In an embodiment, the one or more microbial taxa is selected from the group consisting of Clostridiales sp, COT-028, Peptostreptococcaceae XI [G-4] sp. COT-019, or Capnocytophaga sp. COT-339, and combinations thereof. In certain embodiments, the one or more microbial taxa is associated with periodontal health or periodontal disease. In certain embodiments, the one or more microbial taxa in the control data set is associated with periodontal health or periodontal disease. In certain embodiments, the one or more microbial taxa is a fungal species, an archaea species, or a protozoan species. In certain embodiments, the one or more microbial taxa is present in a sample

In any of the methods disclosed herein, the animal is a domestic animal. In certain embodiments, the domestic animal is a dog, cat, horse, cow, ferret, rabbit, pig, rat, mouse, gerbil, hamster, or goat. In certain embodiments, the animal is a wild animal. In certain embodiments, the wild animal is a wolf, bison, elk, deer, lion, or tiger. In certain embodiments, the presently disclosed methods further comprise extracting DNA from the sample. In certain embodiments, the sample is a gingival area sample, e.g., gingival margin, a subgingival area sample, a supragingival area sample, a saliva sample, a tongue sample, or a buccal sample. In certain embodiments, wherein the sample is obtained from a conscious animal or from an unconscious animal. In certain embodiments, the animal has or is suspected to have gingivitis and/or periodontitis. In certain embodiments, the oral health status comprises periodontal disease.

In any of the methods disclosed herein, the one or more microbial taxa is quantified using quantitative polymerase chain reaction (qPCR) or DNA sequencing methods. In an embodiment, the abundance or relative abundance of the one or more microbial taxa is determined by amplifying and/or sequencing 16S rRNA, 16S rDNA, ITS, 18S rRNA, or 18S rDNA. In certain embodiments, the bacterial taxa are determined by amplifying and/or sequencing one or more of the V1-V3, V3-V4, or V4 regions of the 16S rDNA, such as by using 454-pyrosequencing or Illumina sequencing.

In any of the methods disclosed herein, the detection of the presence of bacteria or other markers can include measuring the amounts of bacteria or other markers, and the amounts can be compared to a scale that correlates the amount of bacteria or other markers to the likelihood that the animal has oral disease or disorder (e.g., periodontal disease) or poor oral health. The likelihood can be indicated as a percentage. For purpose of example and not limitation, the Cq (cycle quantitation) score of a qPCR test that detects the nucleic acid (e.g. DNA or RNA) of bacteria associated with oral disease or disorder (e.g., periodontal disease) can be used to create the scale for calculating the likelihood that the animal has oral disease or disorder (e.g., periodontal disease). A lower Cq score can indicate the presence of higher levels of the bacteria associated with oral disease or disorder (e.g., periodontal disease) and therefore the likelihood that the animal has oral disease or disorder (e.g., periodontal disease) can be higher than the animal with a higher Cq score.

In certain exemplary embodiments, all qPCR data can be normalized to the level of a universal assay for each sample; this adjusts the data for differences in the overall amount of total bacterial DNA in each sample, i.e., yield the abundance relative to the total bacterial population. The data can be then linearised, such that the final qPCR data outputs are relative proportions (2^{-(Cq.Test-Cq.Total)}). In certain embodiments, Cq. Test refers to the Cq score associated with a microbial species. In certain embodiments, Cq. Test refers to the Cq score associated with a canine oral taxon (COT). Samples with Cq.Test values outside of the reliable range of the assay (where Cq>21) can be assumed to have undetectable amounts of DNA and therefore those relative proportions can be imputed as 0. Cq and Ct (cycle threshold) can be used interchangeably.

For purpose of example and not limitation, a report can be generated summarizing the results of sample testing. In certain embodiments, electronic communications can be used to communicate the report. For example, a personalized report can be generated and sent to communicate the animal's oral health status. In other embodiments, the report can be provided as a hard copy. The personalized report can, for example, include an indicator system such as a traffic light system, e.g., green, yellow, red, to communicate the oral health status of the animal. The personalized report can also include a representation of the scale as reference above and an indication of where the animal's oral health falls on the scale, e.g., 0% is indicative of no disease and 100% is indicative of severe disease.

### 5. MACHINE LEARNING MODEL

In certain embodiments, the present disclosure also provides machine learning models for detecting one or more microbial taxa (e.g., bacteria) in an oral microbiome of an animal. The one or more microbial taxa (e.g., bacteria) can be associated with an oral disease or disorder, e.g., periodontal disease, or with good oral health. The animal can be a companion animal, such as a dog or a cat. In certain embodiments, the one or more microbial taxa are one or more bacteria.

The disclosed methods can include methods implemented by a computer system for determining the animal oral health state. In certain embodiments, the methods comprise receiving input data for an animal, wherein the input data comprises at least a first array comprising a first plurality of entries, and each entry comprises a numerical value that indicates an amount or relative abundance of a type of one or more microbial taxa; determining, by a machine learning model, an animal oral health state based on the input data for the animal, wherein the animal oral health state identifies a predicted oral health state classification for the animal; obtaining the animal oral health state from the machine learning model; and outputting the animal oral health state. In certain embodiments, the input data comprises quantified one or more microbial taxa associated with periodontal health or periodontal disease from the sample. In certain embodiments, the one or more microbial taxa is quantified using qPCR or DNA sequencing methods. In certain embodiments, the bacterial taxa are determined by amplifying and/or sequencing one or more of the V1-V3, V3-V4, or V4 regions of the 16S rDNA, such as by using 454-pyrosequencing or Illumina sequencing. In certain embodiments, the one or more microbial taxa in the control data set is associated with periodontal health or periodontal disease. In certain embodiments, the methods further comprise: obtaining training data for a plurality of animals, wherein the training data indicates an amount or relative abundance of a type of microorganism that is present within a sample for each animal from among the plurality of animals; associating the training data with animal oral health state classifications, wherein associating the training data with the animal oral health state classifications comprises associating each animal from among the second plurality of animals with an animal oral health state classification; and training the machine learning model using the training data that is associated with the animal oral health state classifications. In certain embodiments, the methods further comprise: identifying, by the machine learning model, particular microbial taxa in canine plaque that are significantly associated with health, gingivitis, and/or periodontitis for determining the oral health state of the animal. In certain embodiments, the input data further comprises one or more of an animal breed identifier, an animal size, an animal weight, an animal age, animal health information, animal diet, a geographical location information, a sample location, or a combination thereof. In certain embodiments, the input data comprise abundance or relative abundance of the one or more microbial taxa. In certain embodiments, the one or more microbial taxa are bacterial species.

In certain embodiments, the one or more microbial taxa is selected from the group consisting of Actinomyces sp., Anaerovorax sp., Bacteroides sp., Bergeyella sp., Capnocytophaga sp., Clostridiales sp., Desulfomicrobium sp., Filifactor sp., Frigovirgula sp, Fusobacterium sp, Gemella sp., Granulicatella sp., Helcococcus sp., Lachnospiraceae sp., Leptotrichia sp., Moraxella sp., Neisseria sp., Odoribacter sp., Pasteurellaceae sp., Peptococcus sp., Peptostreptococcaceae sp., Peptostreptococcus sp. Porphyromonas sp., Synergistales sp., Tannerella sp., and combinations thereof. In certain embodiments, the one or more microbial taxa is selected from the group consisting of Actinomyces sp. COT-083, Actinomyces sp. COT-252, Anaerovorax sp. COT-125, Bacteroides sp. COT-040, Bergeyella zoohelcum COT-186, Capnocytophaga sp. COT-339, Clostridiales sp. COT-005, Clostridiales sp. COT-028, Desulfomicrobium orale COT-008, Filifactor sp. COT-064, Frigovirgula sp. COT-007, Fusobacterium sp. COT-169, Fusobacterium sp. COT-189, Gemella palaticanis COT-089, Granulicatella sp. COT-095, Helcococcus sp. COT-069, Lachnospiraceae XIVa [G-4] sp. COT-099, Lachnospiraceae XIVa [G-5] sp. COT-024, Leptotrichia sp. COT-345, Moraxella sp. COT-017, Moraxella sp. COT-018, Neisseria animaloris COT-016, Odoribacter denticanis COT-084, Pasteurellaceae sp. COT-271, Peptococcus sp. COT-044, Peptostreptococcaceae XI [G-1] sp. COT-004, Peptostreptococcaceae XI [G-1] sp. COT-006, Peptostreptococcaceae XI [G-3] sp. COT-104, Peptostreptococcaceae XI [G-4] sp. COT-019, Peptostreptococcaceae XI [G-6] sp. COT-068, Peptostreptococcaceae XIII [G-1] sp. COT-030, Peptostreptococcaceae XIII [G-2] sp. COT-077, Peptostreptococcus sp. COT-033, Peptostreptococcus sp. COT-227, Porphyromonas cangingivalis COT-109, Porphyromonas gingivicanis COT-022, Porphyromonas gulae I COT-052, Porphyromonas macacae COT-192, Porphyromonas sp. COT-108, Synergistales [F-2,G-1] sp. COT-138, Tannerella forsythus COT-023, and combinations thereof. In certain embodiments, the one or more microbial taxa is selected from the group consisting of Clostridiales sp, COT-028, Peptostreptococcaceae XI [G-4] sp. COT-019, Capnocytophaga sp. COT-339, and combinations thereof. In certain embodiments, the one or more microbial taxa is a fungal species, an archaea species, or a protozoan species. In certain embodiments, the abundance or relative abundance of the one or more microbial taxa is determined by amplifying 16S rRNA, 16S rDNA, ITS, 18S rRNA, or 18S rDNA. In certain embodiments, the one or more microbial taxa is associated with periodontal health or periodontal disease. In certain embodiments, the one or more microbial taxa in the control data set is associated with periodontal health or periodontal disease. In certain embodiments, the one or more microbial taxa is present in a sample.

In certain embodiments, the animal is a domestic animal. In certain embodiments, the domestic animal is a dog, cat, horse, cow, ferret, rabbit, pig, rat, mouse, gerbil, hamster, or goat. In certain embodiments, the animal is a wild animal. In certain embodiments, the wild animal is a wolf, bison, elk, deer, lion, or tiger. In certain embodiments, the sample is a gingival area sample, a gingival margin sample, a subgingival area sample, a supragingival area sample, a saliva sample, a tongue sample, or a buccal sample. In certain embodiments, the sample is obtained from a conscious animal or from an unconscious animal. In certain embodiments, the animal has or is suspected to have gingivitis and/or periodontitis.

In another aspect, the present disclosure provides a system comprising: one or more non-transitory computer-readable storage media including instructions; and one or more processors coupled to the storage media, the one or more processors configured to execute the instructions to: receive input data for an animal, wherein the input data comprises at least a first array comprising a first plurality of entries, and each entry comprises a numerical value that indicates an amount of a type of one or more microbial taxa; determine, by a machine learning model, an animal oral health state based on the input data for the animal, wherein the animal oral health state identifies a predicted oral health state classification for the animal; obtain the animal oral health state from the machine learning model; and output the animal oral health state.

In another aspect, the present disclosure provides a system comprising one or more non-transitory computer-readable storage media including instructions; and one or more processors coupled to the storage media, the one or more processors configured to execute instructions to perform any of the methods disclosed herein.

In a further aspect, the present disclosure provides a non-transitory computer-readable medium comprising: instructions that, when executed by one or more processors of a computing system, cause the one or more processors to: receive input data for an animal, wherein the input data comprises at least a first array comprising a first plurality of entries, and each entry comprises a numerical value that indicates an amount of a type of one or more microbial taxa; determine, by a machine learning model, an animal oral health state based on the input data for the animal, wherein the animal oral health state identifies a predicted oral health state classification for the animal; obtain the animal oral health state from the machine learning model; and output the animal oral health state.

In a further aspect, the present disclosure provides a non-transitory computer-readable medium comprising instructions that, when executed by one or more processors of a computing system, cause the one or more processors to perform any of the methods disclosed herein.

### System Overview

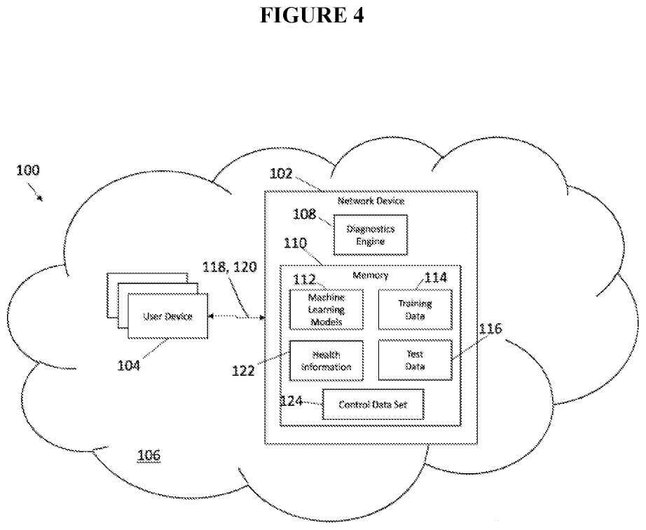

FIG. 4 is a schematic diagram of an embodiment of a diagnostic system 100 that is configured to perform animal diagnostics using machine learning. The diagnostic system 100 is generally configured to input various types of information that are associated with the health and attributes of an animal into a machine learning model 112. The machine learning model 112 is configured to predict an oral health state of the animal based on the provided inputs. This process allows the diagnostic system 100 to determine the oral health state of an animal based on the physical attributes of the animal.

In one embodiment, the diagnostic system 100 comprises one or more user devices 104 and a network device 102 that are in signal communication with each other over a network 106. The network 106 can be any suitable type of wireless and/or wired network including, but not limited to, all or a portion of the Internet, an Intranet, a private network, a public network, a peer-to-peer network, the public switched telephone network, a cellular network, a local area network (LAN), a metropolitan area network (MAN), a personal area network (PAN), a wide area network (WAN), and a satellite network. The network 106 can be configured to support any suitable type of communication protocol as would be appreciated by one of ordinary skill in the art.

### User Devices

Examples of user devices 104 include, but are not limited to, a computer, a laptop, a tablet, a smartphone, a smart device, an Internet-of-Things (IoT) device, a data storage device (e.g., a Universal Serial Bus (USB) drive or flash drive), or any other suitable type of device. A user device 104 is configured to provide input data 118 for an animal to the network device 102. The input data 118 can comprise information associated with bacterial, fungal, or archaea information such as taxa or genetic information, an animal breed identifier, an animal size, an animal weight, an animal age, animal health information (e.g., veterinary records), animal diet, geographical location information, or any other suitable type of information that is associated with an animal. In response to providing the input data 118 for an animal to the network device 102, the user device 104 is configured to receive an animal oral health state classification from the network device 102 and to display the animal oral health state classification to a user. For example, the user device 104 can comprise a graphical user interface (e.g., a display or a touchscreen) that allows a user to view the animal oral health state classification. The user device 104 can further comprise a touchscreen, a touchpad, keys, buttons, a mouse, or any other suitable type of hardware that allows a user to provide inputs into the user device 104. In some embodiments, the oral health state classification can include health (H or PD0), gingivitis (G or PD1), and periodontitis (periodontal disease stages 2, 3, or 4; PD2, PD3, or PD4), e.g., as described by AVDC.

### Network Device

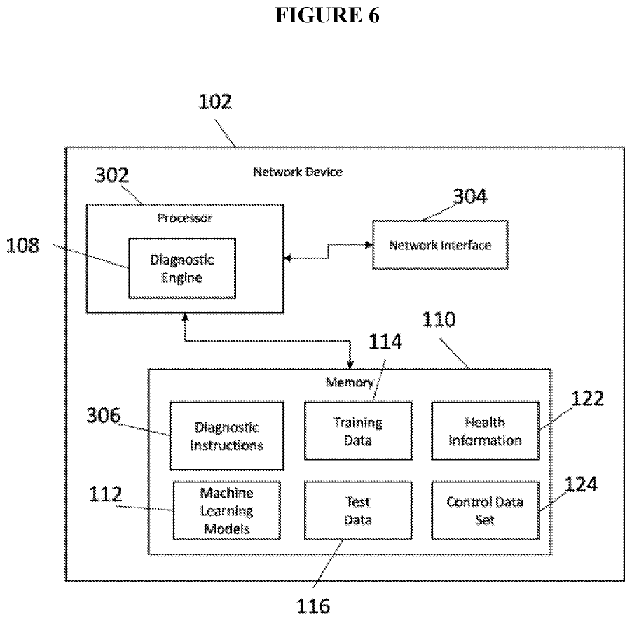

Examples of the network device 102 include, but are not limited to, a server (e.g., a cloud server), a computer, a laptop, or any other suitable type of network device. In one embodiment, the network device 102 comprises a diagnostics engine 108 and a memory 110. Additional details about the hardware configuration of the network device 102 are described in FIG. 6. The memory 110 is configured to store machine learning models 112, training data 114, test data 116, health information 122, a control data set 124, and/or any other suitable type of data.

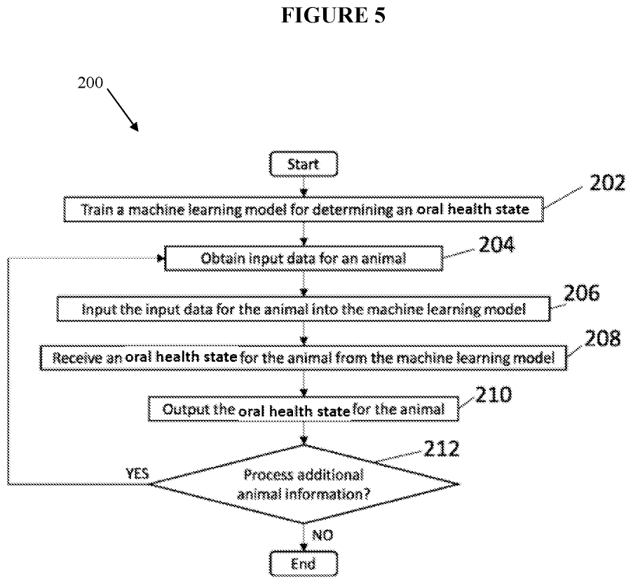

In one embodiment, the diagnostics engine 108 is generally configured to employ a machine learning model 112 to determine an animal's oral health state based on information that is associated with an animal. An example of the diagnostics engine 108 in operation is described in more detail below in FIG. 5.

Examples of machine learning model types include, but are not limited to, a multi-layer perceptron, a recurrent neural network (RNN), an RNN long short-term memory (LSTM), a convolutional neural network (CNN), deep learning algorithms, probabilistic models, a linear regression, a non-linear regression, or any other suitable type of algorithm or model, e.g., logistic regression (LR), weighted k-nearest neighbour (KKNN), kernel support vector machines (KSVM), linear discriminant analysis (LDA), and random forest (RF). The machine learning model 112 can be configured with any suitable type of hyperparameters or settings. Examples of hyperparameters and settings include, but are not limited to, a sensitivity level, a tolerance level, an epoch value, a number of layers (e.g., hidden layers), a number of inputs, a number of outputs, an output type, an output format, or any other suitable type or combination of settings. As an example, the machine learning model 112 can be configured with hyperparameters such as a learning rate of 0.15, a max depth of 2, and a maximum number of rounds set to 22. As another example, the machine learning model 112 can be configured with hyperparameters such as a learning rate of 0.15, a max depth of 5, and a maximum number of rounds set to 32. In other examples, the machine learning model 112 can be configured with any other suitable hyperparameters.

The machine learning model 112 is generally configured to receive input data 118 for an animal as an input and to output an animal oral health state classification 120 based on the provided input data 118. The animal oral health state classification 120 is a classification (e.g., health (H or PD0), gingivitis (G or PD1), and periodontitis (stage 2; PD2, stage 3; PD3 or stage 4; PD4)) that corresponds with a predicted oral health state for the animal based on the provided input data 118. Additional information on the health state classification can be found in the American Veterinary Dental College (AVDC) website (www.avdc.org). In one embodiment, the machine learning model 112 is trained using supervised learning with training data 114 that comprises information associated with different animals with their corresponding labels (e.g., animal oral health state classification 120). During the training process, the machine learning model 112 determines weights and bias values that allow the machine learning model 112 to map information associated with different animals to different animal oral health state classification 120. Through this process, the machine learning model 112 is able to identify an animal oral health state classification 120 based on the provided input data 118. The diagnostics engine 108 can be configured to train the machine learning model 112 using any suitable technique as would be appreciated by one of ordinary skill in the art. For example, the machine learning model 112 can be trained using an XGBoost algorithm. In some embodiments, the machine learning model 112 can be stored and/or trained by a device that is external from the network device 102.

In some embodiments, the network device 102 can be configured to use statistical models, regression models (e.g., non-linear regression models), parametric models, or any other suitable type of model with or in place of the machine learning model 112.

The control data set 124 can comprise information associated with microbial taxa, an animal breed identifier, an animal size, an animal weight, an animal age, animal health information, animal diet, geographical location information, or any other suitable type of information that is associated with a plurality of animals. For example, the control data set 124 can comprise information about the oral microbiome of canids at different oral health status.

The training data 114 can comprise information associated with microbial taxa, an animal breed identifier, an animal size, an animal weight, an animal age, animal health information, animal diet, geographical location information, sample location (e.g., gingival margin, supragingival, or subgingival), or any other suitable type of information that is associated with an animal that can be input into a machine learning model 112. For example, the training data 114 can comprise at least a portion of the control data set 124 that is collected for a plurality of animals. The test data 116 is the same data type as the training data 114. In some embodiments, the test data 116 is a subset or a portion of the training data 114. For example, twenty percent of the training data 114 can be used as test data 116. In other examples, any other suitable percent of the training data 114 can be used as test data 116.

The health information 122 comprises information that is associated with one or more animals. Examples of health information include, but are not limited to, contact information for an owner of an animal, an animal name or identifier, information associated with microbial taxa, an animal breed identifier, DNA information, an animal size, an animal weight, an animal age, animal health information, animal diet, geographical location information, gingivitis information, periodontitis information, or any other suitable type of information that is associated with an animal.

### Oral Health State Determination Process Using Machine Learning

FIG. 5 is a flowchart of an embodiment of an oral health state determination process 200 for the diagnostic system 100. The diagnostic system 100 can employ process 200 to predict the oral health state of an animal using machine learning. Process 200 allows the diagnostic system 100 to input various types of information that are associated with the health and physical attributes of an animal into a machine learning model 112 that is configured to predict the oral health state of the animal. This process allows the diagnostic system 100 to determine the oral health state of an animal based on the physical attributes of the animal.

At step 202, before employing the machine learning model 112 to determine an animal oral health state classification 120 for an animal, the network device 102 first trains the machine learning model 112 for determining an animal oral health state classification 120. During the training process, the machine learning model 112 determines weights and bias values that allow the machine learning model 112 to map certain types of training data 114 to different types of animal oral health state classifications 120. In one embodiment, the machine learning model 112 is trained using a supervised learning training process using labelled training data 114. The supervised learning training process can comprise obtaining training data 114 for a plurality of animals, associating the training data 114 for each animal with an animal oral health state classification 120, and then training the machine learning model 112 using the training data 114 that is associated with the animal oral health state classification 120. Associating the training data 114 with the animal oral health state classification 120 links the metadata for each animal with its corresponding animal oral health state classification 120. After training, each machine learning model 112 is configured to receive microbial taxa, an animal breed identifier, an animal size, an animal weight, an animal age, animal health information, animal diet, geographical location information, sample location (e.g., gingival margin, supragingival, or subgingival), or any other suitable type of information that is associated with an animal as an input and to output an animal oral health state classification 120 based on the input data 118. Through this process, each machine learning model 112 is trained to predict an animal's oral health state (i.e., an animal oral health state classification 120) based on the input data 118. The network device 102 can be configured to train the machine learning model 112 using any suitable technique. In some embodiments, the machine learning model 112 can be trained by a third-party device (e.g., a cloud server) that is external from the network device 102. After training the machine learning model 112, the machine learning model 112 is stored in memory (e.g. memory 110). This concludes the training process for the machine learning model 112.

At step 204, the network device 102 obtains input data 118 for an animal. In one embodiment, the network device 102 can obtain the input data 118 from a user device 104. For example, the user device 104 can send or transfer the input data 118 to the network device 102 as a message or a data file. In this example, the user device 104 can send or transfer the input data 118 to the network device 102 using any suitable messaging or data transfer technique. In some embodiments, a user can directly provide the input data 118 to the network device 102. For example, the user can enter (e.g., type) the input data 118 into the network device 102 using a user interface (e.g., keyboard, mouse, and/or touch screen) on the network device 102. The input data 118 can comprise any suitable combination of microbial taxa, an animal breed identifier, an animal size, an animal weight, an animal age, animal health information, animal diet, geographical location information, sample location (e.g., gingival margin, supragingival, or subgingival), or any other information associated with the animal.

In one embodiment, the input data 118 comprises an array of microbial taxa values. For example, the bacterial taxa can be determined using qPCR amplification or sequencing of 16S rDNA, bacterial genomes, or archaeal genomes. These methods identify the type and/or amount of bacteria that are present within a sample that is collected from the mouth of the animal. The sample can comprise bacteria from a gingival area (e.g., near the gums), subgingival area (e.g., below the gum line), supragingival (e.g., above the gum) in the mouth of the animal, saliva, tongue, and buccal samples. Additional details for the process of collecting a sample and identifying bacterial taxa from within the sample are also provided below. For example, the input data 118 can comprise an array that is associated with different types of bacteria that can be present within the mouth of the animal. The array comprises a plurality of entries that are each associated with a particular type of bacteria. In this example, a value of zero for an entry indicates that the type of bacteria that is associated with the entry was not present or detected within the mouth of the animal. A value of one for an entry indicates that the type of bacteria that is associated with the entry was present or detected within the mouth of the animal. In some embodiment, an entry can comprise a numeric value that indicates the amount of the bacteria for each bacteria type that was present or detected within the mouth of the animal. In other embodiments, a bacterial taxa amount such as a Cq value can be a numeric value or code that uniquely identifies a bacteria type that is present in the mouth of the animal. For example, each bacteria type can be linked with a unique numerical value or code. In other embodiments, the bacterial taxa can use any other suitable type of format or data structure to identify a bacteria type that is present in the mouth of the animal. In one embodiment, the bacterial taxa can comprise one or more, preferably two, bacterial taxa. In some embodiment, the bacterial taxa are preferably collected from a subgingival portion of the mouth.

In one embodiment, the input data 118 comprises an array of eukaryotic taxa values. For example, the eukaryotic taxa can be determined using qPCR amplification or sequencing of eukaryotic microorganism genomes, e.g., fungal genomes, or protozoan genomes.

In some embodiments, the microbial taxa can be collected from an animal while it is conscious. In this case, the microbial taxa can be collected from a gingival margin, a subgingival area, a supragingival area, saliva, tongue, or a buccal sample from the mouth of the animal. In some embodiments, the microbial taxa can be collected from an animal while it is unconscious. In this case, the microbial taxa can be collected from a gingival margin, a subgingival area, a supragingival area, saliva, tongue, or a buccal sample from the mouth of the animal.

In some embodiments, the input data 118 can further comprise an animal breed identifier that identifies a breed of the animal. Examples of animal breeds include, but are not limited to, Affenpinscher, Australian Silky Terrier, Bichon Frise, Bolognese, Cavalier King Charles Spaniel, Chihuahua, Chinese Crested, Coton De Tulear, English Toy Terrier, Griffon Bruxellois, Havanese, Italian Greyhound, Japanese Chin, King Charles Spaniel, Lowchen (Little Lion Dog), Maltese, Miniature Pinscher, Papillon, Pekingese, Pomeranian, Pug, Russian Toy, Yorkshire Terrier, French Bulldog, Beagle, Dachshund, Pembroke Welsh Corgi, Miniature Schnauzer, Cavalier King Charles Spaniel, Shih Tzu, Boston Terrier, Bulldog, Cocker Spaniel, Shetland Sheepdog, Border Collie, Basset Hound, Siberian Husky, Dalmatian, Great Dane, Neapolitan mastiff, Scottish Deerhound, Dogue de Bordeaux, Newfoundland, English mastiff, Saint Bernard, Leonberger and Irish Wolfhound, and cross-breeds. In one embodiment, the animal breed type can be identified using one-hot encoding. For example, the input data 118 can comprise an array that is associated with different breeds of the animal. The array comprises a plurality of entries that are each associated with a particular breed type. In this example, a value of zero for an entry indicates that the animal is not a member of the breed type that is associated with the entry. A value of one for an entry indicates that the animal is a member of the breed type that is associated with the entry. In other embodiments, the animal breed identifier can be a numeric value or code that uniquely identifies a breed type. For example, each breed type can be linked with a unique numerical value. In other embodiments, the animal breed identifier can use any other suitable type of format or data structure to identify a breed type for the animal.

In some embodiments, the input data 118 can further comprise an animal size classification value. The animal size classification value identifies the size of the animal based on the physical size and/or weight of the animal. Examples of animal sizes include, but are not limited to, toy/extra-small breeds, extra-small breeds, small breeds, medium-small breeds, medium breeds, medium-large breeds, large breeds, and giant/extra-large breeds. As an example, a toy/extra-small breed can correspond with animals that are physically smaller than small breed animals. A small breed can correspond with animals that have an average body weight of from about 6.5 kilograms to about 9 kilograms. A medium breed can correspond with an animal that has an average body weight between about 9 kilograms and about 30 kilograms. A large breed can correspond with an animal that has an average body weight of between about 30 kilograms and about 40 kilograms. A giant breed can correspond with an animal that has an average body weight of between over about 40 kilograms. Additional details on the animal size and breed classification can be found in Section 3 of the present disclosure. In one embodiment, the animal size classification value can be identified using one-hot encoding. For example, the input data 118 can comprise an array that is associated with different animal size classifications. The array comprises a plurality of entries that are each associated with a particular animal size. In this example, a value of zero for an entry indicates that the animal is not a member of the animal size classification (e.g., toy/extra-small breed, small breed, medium breed, or large breed) that is associated with the entry. A value of one for an entry indicates that the animal is a member of the animal size classification that is associated with the entry. In other embodiments, the animal size classification value can be a numeric value or code that uniquely identifies an animal size classification. For example, each animal size classification can be linked with a unique numerical value. In other embodiments, the animal size classification value can use any other suitable type of format or data structure to identify an animal size classification for the animal. In one embodiment, the input data 118 can comprise the animal size classification value and one or more, preferably two, microbial taxa. In some embodiment, the microbial taxa are preferably collected from a gingival margin, subgingival, or supragingival portion of the mouth.

In some embodiments, the input data 118 can further comprise a sample location value that identifies a location in the mouth where a sample was collected for the animal. For example, the sample location value can comprise a numeric value that corresponds with a gingival margin location, a subgingival location, a supragingival location, saliva, tongue, a buccal sample, or a combination thereof.

In some embodiments, the input data 118 can further comprise a weight value that identifies a weight for the animal. For example, the weight value can comprise a numeric value that corresponds with the weight of the animal in pounds or kilograms. In other examples, the weight value can be in any other suitable of units.

In some embodiments, the input data 118 can further comprise a gingivitis value for the animal. The gingivitis value is a numeric value that is associated with a time to bleeding in the gums of the animal when probing the mouth of the animal. In some instances, the gingivitis value can be an average value that is associated with a plurality of teeth in the mouth of the animal. In some instances, the gingivitis value can be based on a visual inspection for inflamed gums.

In some embodiments, the input data 118 can further comprise a periodontitis value for the animal. The periodontitis value is a numeric value that is associated with the amount of periodontitis that is present in the mouth of the animal. For example, the periodontitis value can correspond with a periodontitis stage as defined by the American Veterinary Dental College (AVDC) or the number/proportion of teeth in the mouth with periodontitis. In some instances, the periodontitis value can be based on a visual inspection for gingival recession (e.g., receding gums), furcation exposure (e.g., root exposure), or mobile or missing teeth.

In some embodiments, the input data 118 can further comprise geographic location information that identifies a physical location that is associated with the animal. For example, the geographic location information can identify a country or region where the animal is physically located. For instance, the geographic location information can identify a country such as China, Thailand, the United Kingdom, the United States of America, etc. In other embodiments, the input data 118 can further comprise any other suitable type or combination of information that is associated with the animal. In one embodiment, the geographic location information can be identified using one-hot encoding. For example, the input data 118 can comprise an array that is associated with the geographic location information. The array comprises a plurality of entries that are each associated with a particular country or region. In this example, a value of zero for an entry indicates that the animal is not located within a country or region that is associated with the entry. A value of one for an entry indicates that the animal is located within a country or region that is associated with the entry. In other embodiments, the geographic location information can be a numeric value or code that uniquely identifies a particular country or region. For example, each country and region can be linked with a unique numerical value. In other embodiments, the geographic location information can use any other suitable type of format or data structure to identify a physical location for the animal.

At step 206, the network device 102 inputs the input data 118 for the animal into the machine learning model 112. Here, the network device 102 inputs any suitable combination of information from the input data 118 that was obtained in step 204 into the machine learning model 112. For example, the network device 102 can input the input data 118 as a sequential or parallel combination of arrays or values into the machine learning model 112.

At step 208, the network device 102 receives an animal oral health state classification 120 for the animal from the machine learning model 112. The machine learning model 112 is configured to predict an animal oral health state classification for the animal based on the microbial taxa values, the breed of the animal, the size of the animal, the weight of the animal, the health of the animal, the gingivitis value associated with the animal, the periodontitis value associated with the animal, the geographic location information associated with the animal, or any other suitable type of information, or combination of information, thereof. In response to inputting the input data 118 in the machine learning model 112, the network device 102 receives an animal oral health state classification 120 as an output from the machine learning model 112.

At step 210, the network device 102 outputs the animal oral health state classification 120. Here, the network device 102 outputs the animal oral health state classification 120 for a user to view. As an example, the network device 102 can output the animal oral health state classification 120 by displaying the animal oral health state classification 120 on a graphical user interface (e.g., a display). As another example, the network device 102 can output the animal oral health state classification 120 by writing and saving the animal oral health state classification 120 within a document of file. As another example, the network device 102 can output the animal oral health state classification 120 by sending the animal oral health state classification to a user device 104. In this example, the network device 102 can send the animal oral health state classification 120 to the user device 104 as a message, an email, a text document, a file, a link, or in any other suitable format. After receiving the animal oral health state classification 120 from the network device 102, the user device 104 can then display the animal oral health state classification 120 to a user using a graphical user interface (e.g., a display). In other examples, the network device 102 can use any other suitable technique for outputting the animal oral health state classification 120.

At step 212, the network device 102 determines whether to process additional animal information. Here, the network device 102 determines whether there is any more animal information to process for other animals. For example, a user can provide samples to the network device 102 for one or more other animals to process to determine their oral health state classifications. The network device 102 determines to process additional animal information when there are one or more samples remaining to process. The network device 102 returns to step 204 in response to determining to process additional animal information. In this case, the network device 102 returns to step 204 to obtain input data 118 for another animal and to repeat the process of using the machine learning model 112 to determine the oral health state classification of the animal based on the new input data 118. Otherwise, the network device 102 terminates process 200. In this case, the network device 102 determines that there are no more animals to process and terminates process 200.

In some embodiments, the machine learning model can identify particular microbial species in canine plaque that are significantly associated with health, gingivitis, and/or periodontitis for determining the oral health state of the animal.

### Hardware Configuration for the Network Device

FIG. 6 is an embodiment of a network device 102 for the diagnostic system 100. As an example, the network device 102 can be a server or a computer. The network device 102 comprises a processor 302, a memory 110, and a network interface 304. The network device 102 can be configured as shown or in any other suitable configuration.

### Processor

The processor 302 is a hardware device that comprises one or more processors operably coupled to the memory 110. The processor 302 is any electronic circuitry including, but not limited to, state machines, one or more CPU chips, logic units, cores (e.g., a multi-core processor), field-programmable gate array (FPGAs), application-specific integrated circuits (ASICs), or digital signal processors (DSPs). The processor 302 can be a programmable logic device, a microcontroller, a microprocessor, or any suitable combination of the preceding. The processor 302 is communicatively coupled to and in signal communication with the memory 110 and the network interface 304. The one or more processors are configured to process data and can be implemented in hardware or software. For example, the processor 302 can be 8-bit, 16-bit, 32-bit, 64-bit, or of any other suitable architecture. The processor 302 can include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components.

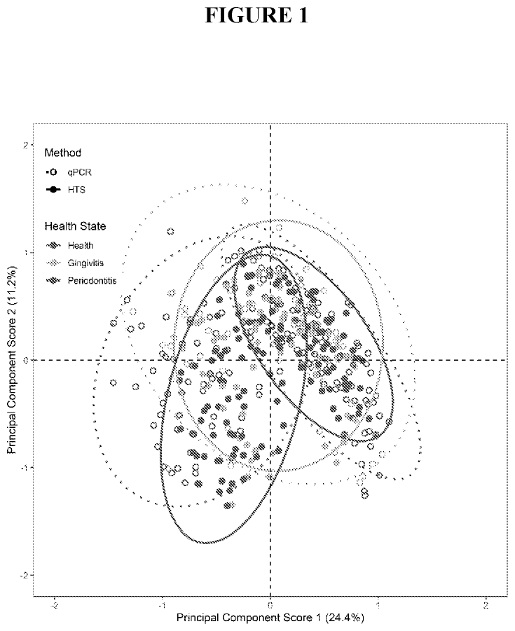

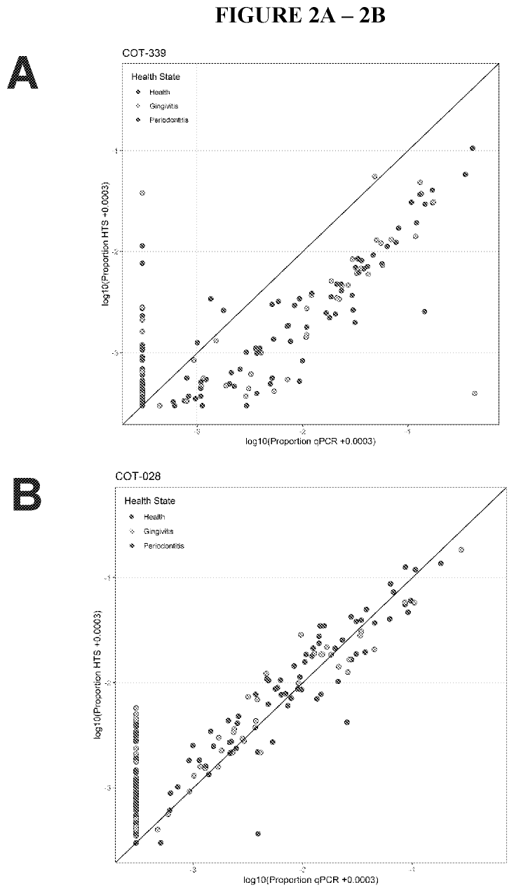

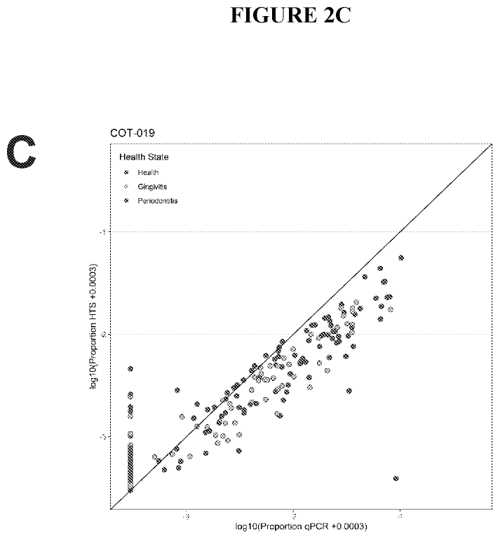

The one or more processors are configured to implement various instructions. For example, the one or more processors are configured to execute diagnostics instructions 306 to implement the diagnostics engine 108. In this way, processor 302 can be a special-purpose computer designed to implement the functions disclosed herein. In an embodiment, the diagnostics engine 108 is implemented using logic units, FPGAs, ASICs, DSPs, or any other suitable hardware. The diagnostics engine 108 is configured to operate as described in FIGS. 1-2. For example, the diagnostics engine 108 can be configured to perform the steps of process 200 as described in FIG. 5.

### Memory

The memory 110 is a hardware device that is operable to store any of the information described above with respect to FIG. 1-6 along with any other data, instructions, logic, rules, or code operable to implement the function(s) described herein when executed by the processor 302. The memory 110 comprises one or more disks, tape drives, or solid-state drives, and can be used as an over-flow data storage device, to store programs when such programs are selected for execution, and to store instructions and data that are read during program execution. The memory 110 can be volatile or non-volatile and can comprise a read-only memory (ROM), random-access memory (RAM), ternary content-addressable memory (TCAM), dynamic random-access memory (DRAM), and static random-access memory (SRAM).

The memory 110 is operable to store diagnostics instructions 306, machine learning models 112, training data 114, test data 116, health information 122, a control data set 124, and/or any other data or instructions. The diagnostics instructions 306 can comprise any suitable set of instructions, logic, rules, or code operable to execute the diagnostics engine 108. The machine learning models 112, the training data 114, the test data 116, the health information 122, and the control data set 124 are configured similar to the machine learning models 112, the training data 114, the test data 116, the health information 122, and the control data set 124 described in FIGS. 4-5, respectively.

### Network Interface

The network interface 304 is a hardware device that is configured to enable wired and/or wireless communications. The network interface 304 is configured to communicate data between user devices 104 and other devices, systems, or domains. For example, the network interface 304 can comprise an NFC interface, a Bluetooth interface, a Zigbee interface, a Z-wave interface, a radio-frequency identification (RFID) interface, a WIFI interface, a LAN interface, a WAN interface, a PAN interface, a modem, a switch, or a router. The processor 302 is configured to send and receive data using the network interface 304. The network interface 304 can be configured to use any suitable type of communication protocol as would be appreciated by one of ordinary skill in the art.

### EXAMPLES

For the purpose of understanding and not limitation, the presently disclosed subject matter will be better understood by reference to the following Example, which is provided as exemplary of the disclosed subject matter, and not by way of limitation.

### Example 1: Diagnostic Value of Species Associated with Periodontal Disease of a companion Animal

The present example evaluated bacterial species of the canine oral microbiome that were associated with periodontal health or disease. These were determined using qPCR measurements relating the abundance of individual species to the total sample, and then applying machine learning models to enable their diagnostic potential to be determined.

### Materials and Methods

### Sampling Strategy and Study Cohort

The sample set used for the study was generated in a previous study described by Davis et al. (2013). Briefly, subgingival plaque samples were harvested from a cohort of 223 dogs, which consisted of 72 with healthy gingiva (clinically normal; periodontitis stage 0, PD0), 77 with gingivitis (gingivitis only without attachment loss; periodontitis stage 1, PD1) and 74 with early periodontitis (attachment loss up to 25%; periodontitis stage 2, PD2), with the stage of periodontitis defined according to the American Veterinary Dental College (AVDC) nomenclature. The subgingival plaque samples were pooled from multiple teeth of the same health state from the same dog. Specific details on these collections including inclusion/exclusion criteria, assignment of clinical health status and associated metadata can be found in Davis et al. (2013).

### High-Throughput Sequencing (HTS) Data

Plaque samples were analysed via 454 pyrosequencing to identify microbial taxa, see description in Davis et al. (2013). Taxonomy was assigned, and the number of sequence reads assigned to a particular taxonomic classification at both species and genus levels determined, as previously described by Davis et al. (2013).

qPCR Assay Development and Validation

qPCR assays were designed based on 16S rRNA sequence information for individual taxa. Taxa selected for assay development were prioritised based on associations with periodontal health or disease, and rankings of presence (% of samples containing the species) and relative abundance (% of species within total bacterial population). All probes were designed with a fluorescein based (FAM) reporter dye, and TaqMan minor groove binder (MGB) or black hole quencher (BHQ), respectively. Briefly, assays were designed using full length consensus 16S sequences from a clone library, developed for a previous study to characterise canine oral microbiota (Dewhirst et al. 2012). Sequences were aligned in Vector NTI with the AlignX tool (Invitrogen™, Thermo Fisher Scientific Inc.) to enable regions of greatest variation to be identified around the 16S rRNA variable regions V1 and V2. Subsequently, Primer Express 3 (Applied Biosystems™, Thermo Fisher Scientific Inc.) was used to generate candidate primer and probe selections.

Performance of all assays was initially assessed via reaction efficiency, determined using a 10-fold dilution series of the amplified target clone DNA. An arbitrary cut-off of ≥80% efficiency was defined as a requirement for an assay to progress to subsequent phases of validation.

Assay specificity was assessed using the clone library with amplified clone DNA pooled into groups of 10 clones using equal amounts of DNA for each clone. Each assay was tested against all clone pools. Any clone pools not containing the target sequence but showing amplification were investigated further with each clone in the pool being tested individually. Cross reactivity against non-target clones was assessed based on the likely maximum proportion of the non-target clone, gauged from the 454 pyrosequencing data. For an assay to be accepted for further use, the contribution of the non-target signal, where the non-target sequence was present at its maximum likely proportion, could not exceed 10%.

qPCR Analysis of Plaque DNA Samples

The qPCR assays were performed on a subset of the same DNA samples, extracted from 205 of the subgingival plaque samples (70 with healthy gingiva, 69 with gingivitis and 66 with early periodontitis) used for HTS (Davis et al., 2013). Briefly, qPCR reactions were conducted using the Biomark system with 48×48 assay chips (Standard BioTools Inc., previously Fluidigm Corporation Ltd). Due to the high throughput nature of the platform, a pre-amplification, enrichment step was conducted. A pre-amplification mixture was prepared, consisting of 25 μl TaqMan™ PreAmp Master Mix (Thermo Fisher Scientific Inc.), 12.5 μl pooled assay mix and 12.5 μl DNA. The pre-amplification conditions were an initial denaturation at 95 °C for 10 min, then 14 cycles with denaturation at 95 °C for 15 s and annealing at 60 °C for 4 min. The main qPCR amplification was then performed on the Biomark instrument, according to the manufacturer's guidelines. Briefly, pre-amplified sample DNA, TaqMan™ Gene Expression Master Mix (Applied Biosystems™, Thermo Fisher Scientific Inc.) and sample loading reagent (Standard BioTools Inc.), mixed with individual qPCR assays were combined for a total volume of 6 μL. Chips were primed, loaded and run according to the manufacturer's instructions.

qPCR Data Analysis

Cq data were exported before analysis with GenEx™ v6.0 (MultiD Analyses AB, Sweden). The same thresholds were applied to all runs of the same qPCR assay across different Biomark chips. Briefly, outlying data points were removed using the outlier test included in GenEx™ which is based on Grubb's test (Grubbs 1969), with options to set the confidence level and cut-off standard deviation (SD). The confidence level was set to 0.10 and the SD cut-off level set to 0.01. Using the previously derived reaction efficiency levels (See Methods: qPCR assay development and validation) for each individual assay, adjustments were made to all data points to account for these differences. The mean was calculated for all replicate data points. All qPCR data were normalised to the level of a universal assay for each sample; this adjusted the data for differences in the overall amount of total bacterial DNA in each sample. The data were then linearised, such that the final qPCR data outputs were relative proportions (2-(Cq.Test-Cq.Total)). In certain embodiments, Cq. Test refers to the Cq score associated with a microbial species. In certain embodiments, Cq. Test refers to the Cq score associated with a canine oral taxon (COT). Samples with Cq. Test values outside of the reliable range of the assay (where Cq>21) were assumed to have undetectable amounts of DNA and therefore those relative proportions were imputed as 0.

Comparison of HTS and qPCR Analysis Technologies

Relative proportions for both HTS and qPCR outputs were log₁₀ transformed (+0.0003 to allow for zeros) prior to analyses. Principal component analysis (PCA) was performed on the log₁₀ proportions to assess the profile of the bacterial species and explore any potential clustering by analysis method or health state. Ellipses representing the 95% bivariate confidence region for PC1 and PC2 were calculated, assuming a multivariate t-distribution (Fox & Weisberg 2011), for each analysis method and health state combination and included on the PCA score plot. Analyses were performed in R v4.2.1 statistical software (R Core Team 2022), using the vegan (Oksanen et al. 2022) and ggplot2 libraries.

The relative proportions of the HTS and qPCR analysis methods were then compared for each of the bacterial species by Pearson's correlation coefficient. In addition, the non-parametric Spearman's rank correlation coefficient was calculated to test the sensitivity of the correlation estimate to the imputed zero relative proportions in the qPCR assay due to the limit of detection.

### Modelling for Microbial Diagnostic Biomarkers

The qPCR data for each microbial taxa was modelled to evaluate single species prediction of periodontitis. Modelling was defined to classify between ‘periodontitis’ (PD2) or ‘not-periodontitis’ (health, PD0 and gingivitis, PD1) samples. Five classification machine learning methods were employed to estimate the diagnostic ability of each species: logistic regression (LR), linear discriminant analysis (LDA), random forest (RF), kernel support vector machines (KSVM) and weighted k-nearest neighbour (KKNN). The models were optimised to maximise the accuracy parameter (sum of correctly classified samples/total number of samples) using 5-fold cross-validation with hyper parameter tuning using a grid search.

Prior to modelling, the samples were split into a training and a test subset with stratification applied to the health state to reduce over specifying the ‘not-periodontitis’ state. Specifically, 72 samples (34 PD2:18 PD1:20 PD0) were used for training the models, and the remaining samples were used for bootstrap sampling the test subset, with 1000 repetitions in the ratio of 35 PD2:20 PD1:20 PD0 samples. The average accuracy, sensitivity (true positive rate: % of periodontitis samples correctly classified as periodontitis) and specificity (true negative rate: % of not-periodontitis samples correctly classified as not-periodontitis) and their standard deviations from those 1000 bootstrap repetitions were then used to estimate the performance of the models.

The machine learning models were performed in R v4.2.1 statistical software, using the mlr (Bischl et al. 2016) library.

### Results

Development and Validation of Single Species qPCR Assays

Based on consolidation of clinical insights developed from a number of cross-sectional and longitudinal HTS studies (Davis et al. 2013, Ruparell et al. 2021, Wallis et al. 2015, Wallis et al. 2021), a portfolio of qPCR assays was developed to enable quantification of specific single species of bacteria (Table 3).

Forty-one qPCR assays each targeting a single bacterial species were successfully developed and validated. The robustness of each qPCR assay was evaluated via efficiency and specificity parameters. Efficiency, a measure of the percentage of target molecules that are copied per PCR cycle, was determined as ≥90% for 32 of the 41 assays (78%) (Table 3). The remaining nine assays (22%) indicated efficiencies between 79.21% and 89.54% (Table 3). The efficiency of Moraxella sp. COT-017, 79.21%, was accepted given its close proximity to the cut-off (≥80%). Specificity of each assay's target detection was established by screening a library comprising 415 clones, representing different bacterial species of canine oral microbiota (Dewhirst et al. 2012). All 41 assays conformed to the threshold criteria, confirming that cross reactivity was sufficiently low to be considered negligible.

TABLE 3
Full and truncated quantitative polymerase chain
reaction (qPCR) assay names and efficiency data.

| | | Assay |
|---|---|---|
| | Truncated | Efficiency |
| Full Assay Name | Assay Name | (%) |
| Actinomyces sp. COT-083 | COT-083 | 92.42 |
| Actinomyces sp. COT-252 | COT-252 | 90.73 |
| Anaerovorax sp. COT-125 | COT-125 | 99.96 |
| Bacteroides sp. COT-040 | COT-040 | 85.76 |
| Bergeyella zoohelcum COT-186 | COT-186 | 97.51 |
| Capnocytophaga sp. COT-339 | COT-339 | 85.86 |
| Clostridiales sp. COT-005 | COT-005 | 95.37 |
| Clostridiales sp. COT-028 | COT-028 | 83.21 |
| Desulfomicrobium orale COT-008 | COT-008 | 95.56 |
| Filifactor sp. COT-064 | COT-064 | 93.29 |
| Frigovirgula sp. COT-007 | COT-007 | 97.55 |
| Fusobacterium sp. COT-169 | COT-169 | 90.18 |
| Fusobacterium sp. COT-189 | COT-189 | 93.22 |
| Gemella palaticanis COT-089 | COT-089 | 89.27 |
| Granulicatella sp. COT-095 | COT-095 | 97.04 |
| Helcococcus sp. COT-069 | COT-069 | 95.79 |
| Lachnospiraceae XIVa [G-4] sp. COT-099 | COT-099 | 97.79 |
| Lachnospiraceae XIVa [G-5] sp. COT-024 | COT-024 | 90.05 |
| Leptotrichia sp. COT-345 | COT-345 | 85.43 |
| Moraxella sp. COT-017 | COT-017 | 79.21 |
| Moraxella sp. COT-018 | COT-018 | 94.32 |
| Neisseria animaloris COT-016 | COT-016 | 90.73 |
| Odoribacter denticanis COT-084 | COT-084 | 95.15 |
| Pasteurellaceae sp. COT-271 | COT-271 | 90.49 |
| Peptococcus sp. COT-044 | COT-044 | 91.92 |
| Peptostreptococcaceae XI [G-1] sp. COT-004 | COT-004 | 96.92 |
| Peptostreptococcaceae XI [G-1] sp. COT-006 | COT-006 | 97.43 |
| Peptostreptococcaceae XI [G-3] sp. COT-104 | COT-104 | 94.77 |
| Peptostreptococcaceae XI [G-4] sp. COT-019 | COT-019 | 95.79 |
| Peptostreptococcaceae XI [G-6] sp. COT-068 | COT-068 | 90.66 |
| Peptostreptococcaceae XIII [G-1] sp. COT-030 | COT-030 | 91.08 |
| Peptostreptococcaceae XIII [G-2] sp. COT-077 | COT-077 | 98.56 |
| Peptostreptococcus sp. COT-033 | COT-033 | 94.88 |
| Peptostreptococcus sp. COT-227 | COT-227 | 96.22 |
| Porphyromonas cangingivalis COT-109 | COT-109 | 99.29 |
| Porphyromonas gingivicanis COT-022 | COT-022 | 89.07 |
| Porphyromonas gulae I COT-052 | COT-052 | 89.54 |
| Porphyromonas macacae COT-192 | COT-192 | 97.51 |
| Porphyromonas sp. COT-108 | COT-108 | 94.96 |
| Synergistales [F-2, G-1] sp. COT-138 | COT-138 | 83.01 |
| Tannerella forsythus COT-023 | COT-023 | 95.64 |

HTS and qPCR Outputs Indicate Good Alignment, Overall and for Individual Assays

To explore the potential of the selected set of taxa for practical application as microbial biomarkers, the relationships between the qPCR assay outputs and the corresponding HTS outputs following analysis of the suite of plaque samples was examined. PCA was used to explore potential variability between the two analysis methods; the first component explained 24.4% and the second component 11.24% of the variability in the qPCR and HTS data (FIG. 1). Discrete clustering was observed by health state with the health and periodontitis samples forming discrete groups in PC1 although there was some overlap. The gingivitis samples were most variable, with the samples dispersed across both the health and periodontitis clusters. This was apparent irrespective of the technology; both were shown to be similar with no distinctive grouping.

To enable species specific insights, the qPCR data from each assay was plotted against the HTS data from Davis et al. (2013) for each corresponding plaque sample. Of the 41 qPCR assays, 30 were strongly correlated (r>0.8) and 10 were moderately correlated (0.8>r>0.5) according to Pearson's correlations (Table 4). According to Spearman's Rank correlations, 25 of the 41 qPCR assays were strongly correlated (rₛ>0.8) and 13 were moderately correlated (0.8>rₛ>0.5) (Table 4). Examples of strongly correlating assays include the periodontal health associated taxa, Capnocytophaga sp. COT-339, and periodontal disease associated taxa, Peptostreptococcaceae XI [G-4] sp. COT-019 and Clostridiales sp. COT-028. For these species, the HTS and qPCR assay technologies were strongly correlated using both Pearson's (r=0.874, r=0.905 and r=0.902 respectively, p<0.001) (Table 4) and Spearman's rank (rₛ=0.805, rₛ=0.902 and rₛ=0.802 respectively, p<0.001) methods (FIGS. 2A-2C). For these three assays, reaction efficiencies of 85.86%, 95.79% and 83.21%, respectively, were determined (Table 3). It is noteworthy that a number of plaque samples indicated zero relative abundance (i.e. below limit of assay detection) with the species specific qPCR probes, but the taxa were detected using HTS.

TABLE 4
Pearson's and Spearman's Rank correlation coefficients between
qPCR and HTS relative proportion data, with their associated p-values

| Truncated | | | Spearman's Rank | |
|---|---|---|---|---|
| Assay | Pearson's Correlation (r) | | Correlation (rₛ) | |
| Name | Coefficient | P-value | Coefficient | P-value |
| COT-083 | 0.890 | <0.001 | 0.922 | <0.001 |
| COT-252 | 0.886 | <0.001 | 0.941 | <0.001 |
| COT-125 | 0.880 | <0.001 | 0.859 | <0.001 |
| COT-040 | 0.630 | <0.001 | 0.578 | <0.001 |
| COT-186 | 0.500 | <0.001 | 0.396 | <0.001 |
| COT-339 | 0.874 | <0.001 | 0.805 | <0.001 |
| COT-005 | 0.043 | 0.541 | −0.004 | 0.953 |
| COT-028 | 0.902 | <0.001 | 0.842 | <0.001 |
| COT-008 | 0.775 | <0.001 | 0.774 | <0.001 |
| COT-064 | 0.876 | <0.001 | 0.790 | <0.001 |
| COT-007 | 0.829 | <0.001 | 0.832 | <0.001 |
| COT-169 | 0.891 | <0.001 | 0.828 | <0.001 |
| COT-189 | 0.687 | <0.001 | 0.743 | <0.001 |
| COT-089 | 0.678 | <0.001 | 0.598 | <0.001 |
| COT-095 | 0.902 | <0.001 | 0.861 | <0.001 |
| COT-069 | 0.929 | <0.001 | 0.890 | <0.001 |
| COT-099 | 0.901 | <0.001 | 0.736 | <0.001 |
| COT-024 | 0.859 | <0.001 | 0.828 | <0.001 |
| COT-345 | 0.868 | <0.001 | 0.781 | <0.001 |
| COT-017 | 0.816 | <0.001 | 0.811 | <0.001 |
| COT-018 | 0.869 | <0.001 | 0.855 | <0.001 |
| COT-016 | 0.831 | <0.001 | 0.816 | <0.001 |
| COT-084 | 0.888 | <0.001 | 0.862 | <0.001 |
| COT-271 | 0.726 | <0.001 | 0.759 | <0.001 |
| COT-044 | 0.838 | <0.001 | 0.836 | <0.001 |
| COT-004 | 0.857 | <0.001 | 0.827 | <0.001 |
| COT-006 | 0.857 | <0.001 | 0.871 | <0.001 |
| COT-104 | 0.762 | <0.001 | 0.600 | <0.001 |
| COT-019 | 0.905 | <0.001 | 0.902 | <0.001 |
| COT-068 | 0.916 | <0.001 | 0.863 | <0.001 |
| COT-030 | 0.952 | <0.001 | 0.880 | <0.001 |
| COT-077 | 0.922 | <0.001 | 0.791 | <0.001 |
| COT-033 | 0.908 | <0.001 | 0.882 | <0.001 |
| COT-227 | 0.866 | <0.001 | 0.639 | <0.001 |
| COT-109 | 0.813 | <0.001 | 0.841 | <0.001 |
| COT-022 | 0.789 | <0.001 | 0.838 | <0.001 |
| COT-052 | 0.673 | <0.001 | 0.680 | <0.001 |
| COT-192 | 0.874 | <0.001 | 0.781 | <0.001 |
| COT-108 | 0.849 | <0.001 | 0.854 | <0.001 |
| COT-138 | 0.544 | <0.001 | 0.420 | <0.001 |
| COT-023 | 0.837 | <0.001 | 0.864 | <0.001 |

Canine Periodontal Disease Associated Species Differ in their Diagnostic Potential

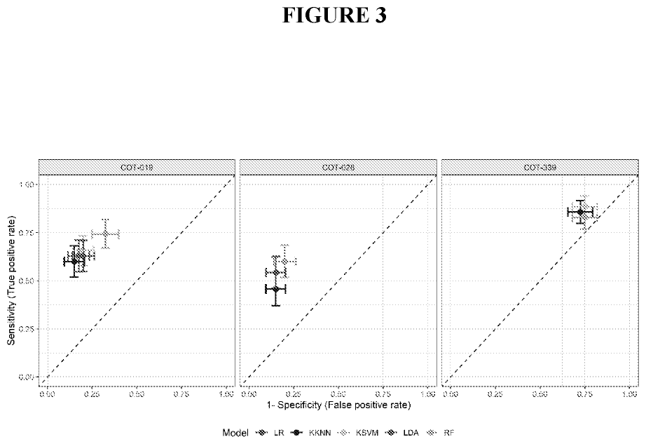

The plaque sample qPCR outputs from assays developed against Capnocytophaga sp. COT-339, Peptostreptococcaceae XI [G-4] sp. COT-019 and Clostridiales sp. COT-028 were modelled to assess their sensitivity (correct classification of periodontitis samples) and specificity (correct classification of non-periodontitis samples) (FIG. 3). For Capnocytophaga sp. COT-339, three models gave outputs with estimated 82.9-88.6% sensitivity and 25.0-27.5% specificity. For Peptostreptococcaceae XI [G-4] sp. COT-019, the outputs of all five models estimated 60.0-74.3% sensitivity and 67.5-85.0% specificity, whereas for Clostridiales sp. COT-028, all five models gave outputs and these estimated 45.7-60.0% sensitivity 80.0-85.0% specificity.

### DISCUSSION

Comprehensive evaluations of the oral plaque microbiota in canine periodontal disease have enabled understanding of the associations of specific bacterial taxa with periodontal health and disease in dogs. Based on these invaluable insights, the inventors of the present disclosure have established a portfolio of more than 40 qPCR assays which selectively target single bacterial species, enabling their relative levels to be accurately quantified from a given plaque DNA sample. Building the panel of assays complements the knowledge of periodontal health status associations with the addition of rigorously validated molecular tools, opening up opportunities for diagnostics to be developed.

The performance of the presently disclosed portfolio of single-species targeted qPCR assays could be assessed by comparing their bacterial detection capability against the equivalent findings delivered via a HTS approach. This indicated moderate to strong overall alignment between the targeted, qPCR approach and the broad-spectrum HTS technology. HTS targets ubiquitous 16S rDNA sequence, thereby promoting amplification of all the members of the microbial community within a given sample. In contrast, the approach undertaken for qPCR assay design exploits novelty in regions of the 16S rRNA sequence to enable targeting of individual bacterial species. The qPCR assays were able to discriminate subgingival plaque samples from dogs with healthy gingiva and periodontitis. This result was similar to the observations from the HTS cross-sectional study which employed the same clinical plaque samples (Davis et al. 2013).

The subsequent evaluation of the two technologies focused on the individual qPCR assays and delivered varying correlation coefficients. Approximately 60% of the validated assays indicated rₛ>0.8 with Spearman's correlation method including those targeting the canine periodontal health associated taxa, Capnocytophaga sp. COT-339, and periodontal disease associated taxa, Peptostreptococcaceae XI [G-4] sp. COT-019 and Clostridiales sp. COT-028. These findings further reinforce the feasibility of a molecular tool such as qPCR to detect microbial biomarkers of canine periodontal disease and provide quantifiable sample-to-sample discrimination comparable to a HTS approach. Scientific publications, additionally, show diversity in the correlation between the two technologies. One investigation concluded substantial agreement (R²=0.872 and R²=0.929) between the methods for targeting cheese microbiota (Dreier et al. 2022). Another comparison, conducted to characterise vaginal lactobacilli reported mixed findings; proportions of one lactobacilli, Lactobacillus crispatus, were well correlated (r=0.79, p<0.001), while that of another, Lactobacillus iners, correlated poorly (r=0.13, p>0.05) (Smidt et al. 2015). Robust correlations between the qPCR and HTS approaches have also been observed in a non-microbial targeted investigation; a faecal-based dietary analysis of Little Penguins located in Western Australia revealed strong correlations (r≥0.973) for four fish species (Murray et al. 2011). In the analysis reported here, it was found that some of the species-specific explorations illustrated that qPCR performance, and associated bacterial detection, was not as sensitive as that observed via HTS. This was most evident for bacterial species present in canine oral plaque at a lower relative abundance. The targeted taxa were determined absent from many samples analysed via the respective qPCR assay, but were detected at quantifiable levels upon assessment with HTS. Whilst qPCR assay design was based on a consensus sequence derived for each bacterial target, in this instance a similarity level of 99%, HTS amplification can be less specific and detect a broader range of related 16S targets. Optimization work with qPCR assay design could result in improved performance and increase the potential of these assays for utilization as diagnostic tools.

The popularity of machine learning approaches is increasing exponentially for numerous scientific applications, with algorithms that can enable more efficient routes to insights, and better decisions regarding best next steps. Here, using qPCR-derived data for three microbial biomarkers, associated with canine periodontal health and disease, machine learning models were utilized to gauge sensitivity and specificity parameters. Similar performance from the five classification model types was found within each assay, indicating stability of the model optimisation. For Capnocytophaga sp. COT-339, the statistical models suggested 82.9-88.6% of periodontitis samples were correctly classified, whilst for non-periodontitis samples 25.0-27.5% were correctly classified. For Clostridiales sp. COT-028, the modelling indicated that 45.7-60.0% of periodontitis samples were correctly identified, whilst the equivalent finding for Peptostreptococcaceae XI [G-4] sp. COT-019 was 60.0-74.3%. Given the disease association identified for both taxa (Davis et al. 2013, Wallis et al. 2015, Ruparell et al. 2021, Wallis et al. 2021), the ability of the COT-019 assay to identify periodontitis samples correctly in a greater proportion of cases is a positive indication. Non-periodontitis was correctly classified for 80.0-85.0% of samples with COT-028 and 67.5-85.0% of samples for COT-019. These indications are more similar between the two assays. In the development of diagnostic tests, there is a trade off in sensitivity and specificity, whereby a test may be good at confirming healthy subjects at the expense of potentially missing disease cases or, alternatively, good at diagnosing disease cases while erroneously describing healthy cases as diseased ones. The consequences of these scenarios need to be considered and the most appropriate balance determined for each type of diagnostic test. Here, the grouping of gingivitis (PD1) with healthy samples (PD0) for non-periodontitis was the same as the categorisation used in the recent investigation by Kwon et al. (2022); these authors termed the health status' in tandem as the reversible group as opposed to the non-reversible group. There are other approaches which could be used for categorisation of samples; however, this one could have particular practical merit. Gingivitis (PD1) can be identified upon visual, conscious assessment, whilst the diagnosis of periodontitis (PD2) typically requires a more detailed investigation and represents the stage in the disease where a diagnostic test could potentially add most value. Sample size is also a consideration and larger sample sets for each category used to build the models will enhance the quality and relevance of the outputs generated. In addition, whilst machine learning methods were applied to the qPCR data generated within this study to understand the diagnostic potential of single species taxa, accuracy parameters could also be estimated for combined multi-species models. Alternatively, similar algorithms could be applied to the existing HTS data to extract additional value from the historical work. Nevertheless, any outputs from machine learning models or laboratory-based assessments with promising diagnostic potential require testing in real world scenarios, where clinical validation studies can confirm that predicted sensitivity and specificity parameters can be achieved.

The methods adopted within this study aim to demonstrate some of the initial steps that could be employed as part of a strategy to develop tools to support periodontal disease diagnosis. Molecular methods such as qPCR offer efficiency in many key areas of consideration for the development of diagnostics including cost, time and requirements for data processing and interpretation compared to other characterisation technologies such as HTS. In addition, whilst the approach illustrated here has focused on qPCR methods targeting single microbial species, high-throughput opportunities could be employed to quantify multiple species in tandem via multiplex qPCR, with the ability to further refine and optimise diagnostic accuracy parameters. Applied in conjunction with conscious plaque sampling and machine learning models, qPCR could therefore provide a tool to help resolve the significant prevalence verses diagnosis gap with canine periodontitis. It is recognized that the current study has been conducted using a subgingival plaque sample set. However, an investigation comparing canine subgingival and gingival margin plaque has shown commonality in the microbiota observed across health and early periodontitis (Ruparell et al. 2021), thus supporting the employment of plaque from above the gum line, and hence conscious sampling, for microbial biomarker-based opportunities. There are existing products in the canine oral health diagnostics arena. One such offering is periodontal diagnostic strips; detecting thiols produced by microbes, however, their mechanism of action is non-specific and not limited solely to periodontal pathogens (Manfra Marretta et al. 2012, Queck et al. 2018). To-date, the most closely aligned work in the field to that described here has been performed by Kwon et al. (2022), with qPCR-based detection of 11 human periodontopathic species, concluding Treponema denticola, a prognostic biomarker for periodontitis in dogs. The exploration highlights the importance of considering the full spectrum of periodontal disease (PD0-4), where possible; this is only partially covered in the study reported here (PD0-2) which utilized a historical sample set focused solely on the reversible and/or manageable spectrum of the disease (Davis et al. 2013). Away from periodontal disease, numerous applications demonstrate the successful, practical employment of qPCR as a diagnostic tool. Prominent examples include zoonotic leptospirosis and leishmaniosis, caused by bacterial Leptospira spp., and protozoa Leishmania infantum and L. donovani, respectively. The literature on qPCR for canine veterinary application is substantial and covers a broad spectrum of epidemiological areas (Flores et al. 2017, Scorza et al. 2021, Smith et al. 2021, Griebsch et al. 2022), as well as discussion of the selection of appropriate sample types (Cavalera et al. 2022, Peris et al. 2021) and the development of detection methods (Fink et al. 2015, Miotto et al. 2018). Within the field of microbiology, canid relevant illustrations of the application of qPCR include the detection of bacterial Ehrlichia spp., for diagnosis of ehrlichiosis (Qurollo et al. 2014, Thomson et al. 2018), and atypical fungus Pneumocystis, a cause of Pneumocystis pneumonia in the immunocompromised (Danesi et al. 2017, Danesi et al. 2022).

The present example reports an approach which could be developed towards a qPCR-based diagnostic tool to detect microbial biomarkers of canine periodontitis in supragingival plaque. Based on strong correlations with HTS data, qPCR assays designed to target specific bacterial species offer an accurate, cost and time efficient strategy with promise for improving diagnosis of this prevalent yet under-reported condition.

### REFERENCES

1. American Veterinary Dental College, AVDC. https://www.avdc.org/.
2. Bischl, B., Lang, M., Kotthoff, L. et al. (2016). mlr: Machine Learning in R. Journal of Machine Learning Research 17, 1-5. http://jmlr.org/papers/v17/15-066.html.
3. Cavalera, M. A., Zatelli, A., Donghia, R., et al. (2022). Conjunctival Swab Real Time-PCR in Leishmania infantum Seropositive Dogs: Diagnostic and Prognostic Values. Biology 11, 184. https://doi.org/10.3390/biology11020184.
4. Danesi, P., Petini, M., Falcaro. C., et al. (2022). Pneumocystis Colonization in Dogs Is as in Humans. International Journal of Environmental Resesearch and Public Health 19, 3192. https://doi.org/10.3390/ijerph19063192
5. Danesi, P., Ravagnan, S., Johnson, L. R., et al. (2017). Molecular diagnosis of Pneumocystis pneumonia in dogs. Medical Mycology 55, 828-842. https://doi.org/10.1093/mmy/myx007.
6. Davis, I. J., Wallis, C., Deusch, O., et al. (2013). A cross-sectional survey of bacterial species in plaque from client owned dogs with healthy gingiva, gingivitis or mild periodontitis. PLOS ONE 8, e83158. https://doi.org/10.1371/journal.pone.0083158.
7. DeBowes, L. J., Mosier, D., Logan, E., et al. (1996). Association of periodontal disease and histologic lesions in multiple organs from 45 dogs. Journal of Veterinary Dentistry 13, 57-60. https://doi.org/10.1177% 2F089875649601300201.
8. Dewhirst, F. E., Klein, E. A., Thompson, E. C., et al. (2012). The canine oral microbiome. PLOS ONE 7, e36067. https://doi.org/10.1371/journal.pone.0036067.
9. Dreier, M., Meola, M., Berthoud, B., et al. (2022). High-throughput qPCR and 16S rRNA gene amplicon sequencing as complementary methods for the investigation of the cheese microbiota. BMC Microbiolology 22, 48. https://doi.org/10.1186/s12866-022-02451-y.
10. Fink, J. M., Moore, G. E., Landau, R., et al. (2015). Evaluation of three 5′ exonuclease-based real-time polymerase chain reaction assays for detection of pathogenic Leptospira species in canine urine. Journal of Veterinary Diagnostic Investigation 27, 159-66. https://doi.org/10.1177% 2F1040638715571360
11. Flores, B. J., Pérez-Sánchez, T., Fuertes, H., et al. (2017). A cross-sectional epidemiological study of domestic animals related to human leptospirosis cases in Nicaragua. Acta Tropica 170, 79-84. https://doi.org/10.1016/j.actatropica.2017.02.031.
12. Fox, J. & Weisberg, S. (2011). An R Companion to Applied Regression. 3^{rd} edn. Thousand Oaks CA: SAGE Publications, Inc http://socserv.socsci.mcmaster.ca/jfox/Books/Companion.
13. Gad, T. (1968). Periodontal disease in dogs. 1. Clinical investigations. Journal of Periodontal Research 3, 268-272. https://doi.org/10.1111/j.1600-0765.1968.tb01929.x.
14. Gorrel, C. (2013) Veterinary Dentistry for the General Practitioner. 2^{nd} edn. Amsterdam, Netherlands: Elsevier Health Sciences.
15. Glickman, L. T., Glickman, N. W., Moore, G. E., et al. (2009). Evaluation of the risk of endocarditis and other cardiovascular events on the basis of the severity of periodontal disease in dogs. Journal of the American Veterinary Medical Association 234, 486-94. https://doi.org/10.2460/javma.234.4.486.
16. Griebsch, C., Kirkwood, N., Ward, M. P., et al. (2022). Emerging leptospirosis in urban Sydney dogs: a case series (2017-2020). Australian Veterinary Journal 100,190-200. https://doi.org/10.1111/avj.13148.
17. Grubbs, F. (1969). Procedures for Detecting Outlying Observations in Samples. Technometrics 11, 1-21.
18. Hall, J. A., Forman, F. J., Bobe, G. et al. (2021). The impact of periodontal disease and dental cleaning procedures on serum and urine kidney biomarkers in dogs and cats. PLOS ONE 16, e0255310. https://doi.org/10.1371/journal.pone.0255310.
19. Harvey, C. E. (1998). Periodontal disease in dogs. Etiopathogenesis, prevalence, and significance.” Veterinary Clinics of North America: Small Animal Practice 28, 1111-1128. https://doi.org/10.1016/S0195-5616 (98) 50105-2.
20. Harvey, C. E., Shofer, F. S., & Laster, L. (1994). Association of age and body weight with periodontal disease in North American dogs. Journal of Veterinary Dentistry 11, 94-105. https://doi.org/10.1177% 2F089875649401100301.
21. Hawkins, S. F. C. & Guest, P. C. (2022). Multiplex Quantitative Polymerase Chain Reaction Diagnostic Test for SARS-COV-2 and Influenza A/B Viruses. Methods in Molecular Biology 2511, 53-65. https://doi.org/10.1007/978-1-0716-2395-4_4.
22. Hoffmann, T. & Gaengler, P. (1996). Epidemiology of periodontal disease in poodles. Journal of Small Animal Practice 37, 309-316. https://doi.org/10.1111/j.1748-5827.1996.tb02396.x.
23. Holcombe, L. J., Patel, N., Colyer, A., et al. (2014). Early canine plaque biofilms: characterization of key bacterial interactions involved in initial colonization of enamel. PLOS ONE 9, e113744. https://doi.org/10.1371/journal.pone.0113744
24. Kortegaard, H. E., Eriksen, T. & Baelum, V. (2008). Periodontal disease in research beagle dogs—an epidemiological study. Journal of Small Animal Practice 49, 610-616. https://doi.org/10.1111/j.1748-5827.2008.00609.x.
25. Kyllar, M. & Witter, K. (2005). Prevalence of dental disorders in pet dogs. Veterinary Medicine-Czech 50, 496-505. https://doi.org/10.17221/5654-VETMED
26. Kwon, D., Bae, K., Kim, H., et al. (2022). Treponema denticola as a prognostic biomarker for periodontitis in dogs. PLOS ONE 17, e0262859. https://doi.org/10.1371/journal.pone.0262859.
27. Lindhe, J., Hamp, S. & Loe, H. (1973). “Experimental periodontitis in the beagle dog.” Journal of Periodontal Research 8, 1-10. https://doi.org/10.1111/j.1600-0765.1973.tb00735.x.
28. Lund, E. M., Armstrong, P. J., Kirk, C. A., et al. (1999). Health status and population characteristics of dogs and cats examined at private veterinary practices in the United States. Journal of the American Veterinary Medical Association 214, 1336-1341.
29. Manfra Marretta, S., Leesman, M., Burgess-Cassler, A., et al. (2012). Pilot evaluation of a novel test strip for the assessment of dissolved thiol levels, as an indicator of canine gingival health and periodontal status. The Canadian Veterinary Journal. 53, 1260-5.
30. Miotto, B. A., da Hora, A. S., Taniwaki, S. A., et al. (2018). Development and validation of a modified TaqMan based real-time PCR assay targeting the lipl32 gene for detection of pathogenic Leptospira in canine urine samples. Brazilian Journal of Microbiology 49, 584-590. https://doi.org/10.1016/j.bim.2017.09.004
31. Murray, D. C., Bunce, M., Cannell, B. L., et al. (2011). DNA-based faecal dietary analysis: a comparison of qPCR and high throughput sequencing approaches. PLOS ONE 6, e25776. https://doi.org/10.1371/journal.pone.0025776.
32. Niemiec, B. A., Gawor, J., Tang, S., et al. (2021). The bacteriome of the oral cavity in healthy dogs and dogs with periodontal disease. Am J Vet Res 83, 50-58. https://doi.org/10.2460/ajvr.21.02.0027.
33. Oksanen, J., Simpson, G., Blanchet, F., et al. (2022). vegan: Community Ecology Package. R package version 2.6-2. https://CRAN.R-project.org/package=vegan.
34. O'Neill, D. G., Church, D. B., P. D. McGreevy, P. D., et al. (2014). Prevalence of disorders recorded in dogs attending primary-care veterinary practices in England. PLOS ONE 9, e90501. https://doi.org/10.1371/journal.pone.0090501.
35. Pavlica, Z., Petelin, M., Juntes, P., et al. (2008). Periodontal disease burden and pathological changes in organs of dogs. Journal of Veterinary Dentistry 25, 97-105. https://doi.org/10.1177% 2F089875640802500210.
36. Pereira Dos Santos, J. D., Cunha, E., Nunes, T., et al. (2019). Relation between periodontal disease and systemic diseases in dogs. Research in Veterinary Science 125, 136-140. https://doi.org/10.1016/j.rvsc.2019.06.007.
37. Pöppl A. G., de Carvalho, G. L. C., Vivian, I. F., et al. (2017). Canine diabetes mellitus risk factors: A matched case-control study. Research in Veterinary Science 114, 469-473. https://doi.org/10.1016/j.rvsc.2017.08.003
38. Peris, M. P., Esteban-Gil, A., Ortega-Hernández. P., et al. (2021). Comparative Study of Real-Time PCR (TaqMan Probe and Sybr Green), Serological Techniques (ELISA, IFA and DAT) and Clinical Signs Evaluation, for the Diagnosis of Canine Leishmaniasis in Experimentally Infected Dogs. Microorganisms 9, 2627. https://doi.org/10.3390/microorganisms9122627.
39. Queck, K. E., Chapman, A., Herzog, L. J., et al. (2018). Oral-Fluid Thiol-Detection Test Identifies Underlying Active Periodontal Disease Not Detected by the Visual Awake Examination. Journal of American Animal Hospital Association 54, 132-137. https://doi.org/10.5326/JAAHA-MS-6607.
40. Qurollo, B. A., Riggins. D., Comyn, A., et al. (2014). Development and validation of a sensitive and specific sodB-based quantitative PCR assay for molecular detection of Ehrlichia species. Journal of Clinical Microbiology 52, 4030-2. https://doi.org/10.1128/JCM.02340-14.
41. R Core Team (2022). R: A language and environment for statistical computing. R Foundation for Statistical Computing, Vienna, Austria. https://www.R-project.org/.
42. Ruparell, A., Wallis, C., Haydock, R., et al. (2021). Comparison of subgingival and gingival margin plaque microbiota from dogs with healthy gingiva and early periodontal disease. Research in Veterinary Science 136, 396-407. https://doi.org/10.1016/j.rvsc.2021.01.011.
43. Santibáñez, R., Rodríguez-Salas, C., Flores-Yáñez, C., et al. (2021). Assessment of Changes in the Oral Microbiome That Occur in Dogs with Periodontal Disease. Veterinary Sciences 8, 291. https://doi.org/10.3390/vetsci8120291.
44. Salt, C., Morris, P. J., German, A. J., et al. (2017). Growth standard charts for monitoring bodyweight in dogs of different sizes. PLOS ONE 12, 9. https://doi.org/10.1371/journal.pone.0182064.
45. Scorza B. M., Mahachi, K. G., Cox, A. C., et al. (2021). Leishmania infantum xenodiagnosis from vertically infected dogs reveals significant skin tropism. PLOS Neglected Tropical Diseases 15, e0009366. https://doi.org/10.1371/journal.pntd.0009366.
46. Smidt I., R. Kiiker, R., Oopkaup, H., et al. (2015). Comparison of detection methods for vaginal lactobacilli. Beneficial Microbes 6,747-51. https://doi.org/10.3920/BM2014.0154.
47. Smith, A. M., Stull, J. W., Evason, M. D., et al. (2021). Investigation of spatio-temporal clusters of positive leptospirosis polymerase chain reaction test results in dogs in the United States, 2009 to 2016. Journal of Veterinary Internal Medicine 35, 1355-1360. https://doi.org/10.1111/jvim.16060.
48. Sorensen, W. P., H. Loe, H. & Ramfjord, S. P. (1980). Periodontal disease in the beagle dog. A cross sectional clinical study. Journal of Periodontal Research 15, 380-389. https://doi.org/10.1111/j.1600-0765.1980.tb00295.x.
49. Thomson, K., Yaaran, T., Belshaw, A., et al. (2018). A new TaqMan method for the reliable diagnosis of Ehrlichia spp. in canine whole blood. Parasites Vectors 11, 350. https://doi.org/10.1186/s13071-018-2914-5.
50. Wallis, C. & Holcombe, L. J. (2020). A review of the frequency and impact of periodontal disease in dogs. Journal of Small Animal Practice 61, 529-540. https://doi.org/10.1111/jsap. 13218.
51. Wallis, C., Marshall, M., Colyer, A., et al. (2015). A longitudinal assessment of changes in bacterial community composition associated with the development of periodontal disease in dogs. Veterinary Microbiology 181, 271-282. https://doi.org/10.1016/j.vetmic.2015.09.003.
52. Wallis, C., Milella, L., Colyer, A., et al. (2021). Subgingival microbiota of dogs with healthy gingiva or early periodontal disease from different geographical locations. BMC Veterinary Research 275, 105717. https:/doi.org/10.1186/s12917-020-02660-5.
53. Wallis, C., Saito, E. K., Salt, C., et al. (2021). Association of periodontal disease with breed size, breed, weight, and age in pure-bred client-owned dogs in the United States. Veterinary Journal 275, 105715. https://doi.org/10.1016/j.tvil.2021.105717.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components can be combined or integrated in another system or certain features can be omitted, or not implemented.

In addition, techniques, systems, subsystems, and methods described and illustrated in the various embodiments as discrete or separate can be combined or integrated with other systems, modules, techniques, or methods without departing from the scope of the present disclosure. Other items shown or discussed as coupled or directly coupled or communicating with each other can be indirectly coupled or communicating through some interface, device, or intermediate component whether electrically, mechanically, or otherwise. Other examples of changes, substitutions, and alterations are ascertainable by one skilled in the art and could be made without departing from the spirit and scope disclosed herein.

1. A method for determining oral health status of an animal comprising:
(a) quantifying one or more microbial taxa from a sample to determine abundance or relative abundance of the one or more microbial taxa,
(b) comparing the abundance or relative abundance of said one or more microbial taxa in the sample to abundance or relative abundance of the microbial taxa in a control data set, and
(c) determining the oral health status of the animal.

2. The method of claim 1, wherein the one or more microbial taxa are bacterial species.

3. The method of claim 2, wherein the one or more microbial taxa is selected from the group consisting of Actinomyces sp., Anaerovorax sp., Bacteroides sp., Bergeyella sp., Capnocytophaga sp., Clostridiales sp., Desulfomicrobium sp., Filifactor sp., Frigovirgula sp, Fusobacterium sp, Gemella sp., Granulicatella sp., Helcococcus sp., Lachnospiraceae sp., Leptotrichia sp., Moraxella sp., Neisseria sp., Odoribacter sp., Pasteurellaceae sp., Peptococcus sp., Peptostreptococcaceae sp., Peptostreptococcus sp. Porphyromonas sp., Synergistales sp., Tannerella sp., and combinations thereof.

4. The method of claim 2 or 3, wherein the one or more microbial taxa is selected from the group consisting of Actinomyces sp. COT-083, Actinomyces sp. COT-252, Anaerovorax sp. COT-125, Bacteroides sp. COT-040, Bergeyella zoohelcum COT-186, Capnocytophaga sp. COT-339, Clostridiales sp. COT-005, Clostridiales sp. COT-028, Desulfomicrobium orale COT-008, Filifactor sp. COT-064, Frigovirgula sp. COT-007, Fusobacterium sp. COT-169, Fusobacterium sp. COT-189, Gemella palaticanis COT-089, Granulicatella sp. COT-095, Helcococcus sp. COT-069, Lachnospiraceae XIVa [G-4] sp. COT-099, Lachnospiraceae XIVa [G-5] sp. COT-024, Leptotrichia sp. COT-345, Moraxella sp. COT-017, Moraxella sp. COT-018, Neisseria animaloris COT-016, Odoribacter denticanis COT-084, Pasteurellaceae sp. COT-271, Peptococcus sp. COT-044, Peptostreptococcaceae XI [G-1] sp. COT-004, Peptostreptococcaceae XI [G-1] sp. COT-006, Peptostreptococcaceae XI [G-3] sp. COT-104, Peptostreptococcaceae XI [G-4] sp. COT-019, Peptostreptococcaceae XI [G-6] sp. COT-068, Peptostreptococcaceae XIII [G-1] sp. COT-030, Peptostreptococcaceae XIII [G-2] sp. COT-077, Peptostreptococcus sp. COT-033, Peptostreptococcus sp. COT-227, Porphyromonas cangingivalis COT-109, Porphyromonas gingivicanis COT-022, Porphyromonas gulae I COT-052, Porphyromonas macacae COT-192, Porphyromonas sp. COT-108, Synergistales [F-2, G-1] sp. COT-138, Tannerella forsythus COT-023, and combinations thereof.

5. The method of claim 2, wherein the one or more microbial taxa is selected from the group consisting of Clostridiales sp, COT-028, Peptostreptococcaceae XI [G-4] sp. COT-019, Capnocytophaga sp. COT-339, and combinations thereof.

6. The method of claim 1, wherein the one or more microbial taxa is a fungal species, an archaea species, or a protozoan species.

7. The method of claim 1, wherein the one or more microbial taxa is quantified using qPCR or DNA sequencing methods.

8. The method of claim 1, wherein the abundance, presence, or relative abundance of the one or more microbial taxa is determined by amplifying or sequencing 16S IRNA, 16S rDNA, ITS, 18S rRNA, or 18S rDNA.

9. The method of any one of claim 1, wherein the animal is a domestic animal.

10. The method of claim 9, wherein the domestic animal is a dog, cat, horse, cow, ferret, rabbit, pig, rat, mouse, gerbil, hamster, or goat.

11. The method of claim 1, wherein the animal is a wild animal.

12. (canceled)

13. The method of claim 1, wherein the one or more microbial taxa is associated with periodontal health or periodontal disease.

14. The method of claim 1, wherein the one or more microbial taxa in the control data set is associated with periodontal health or periodontal disease.

15. The method of claim 1 further comprising extracting DNA from the sample.

16. The method of claim 1, wherein the sample is one or more of a gingival area sample, a gingival margin sample, a subgingival area sample, a supragingival area sample, a saliva sample, a tongue sample, a buccal sample, or a combination thereof.

17. (canceled)

18. The method of claim 1, wherein the animal has or is suspected to have gingivitis and/or periodontitis.

19. The method of claim 1, wherein the oral health status comprises periodontal disease.

20. The method of claim 1, wherein the one or more microbial taxa is present in a sample.

21. A method implemented by a computer system, comprising:
receiving input data for an animal, wherein the input data comprises at least a first array comprising a first plurality of entries, and each entry comprises a numerical value that indicates an amount of a type of one or more microbial taxa;
determining, by a machine learning model, an animal oral health state based on the input data for the animal, wherein the animal oral health state identifies a predicted oral health state classification for the animal;
obtaining the animal oral health state from the machine learning model; and
outputting the animal oral health state.

22.-43. (canceled)

44. A system comprising:
one or more non-transitory computer-readable storage media including instructions; and
one or more processors coupled to the storage media, the one or more processors configured to execute the instructions to:
receive input data for an animal, wherein the input data comprises at least a first array comprising a first plurality of entries, and each entry comprises a numerical value that indicates an amount of a type of one or more microbial taxa;
determine, by a machine learning model, an animal oral health state based on the input data for the animal, wherein the animal oral health state identifies a predicted oral health state classification for the animal;
obtain the animal oral health state from the machine learning model; and
output the animal oral health state.

45. (canceled)

46. (canceled)

47. (canceled)


SEQUENCE LISTING (41 sequences)

SEQ ID NO: 1 - DNA, 1516 bp, Actinomyces sp.
  source 1..1516: mol_type = genomic DNA
gacgaacgctggcggcgtgcttaacacatgcaagtcgaacgggatctgatggggcttttgctttgttggtgagagtggcgaacgggtgagtaacacgtgagtaacctgccctcaacatttgaataactccatgaaagtggggctaatgcaatatattctgcagtcatcgcatggtggttgtgggaaacggttttttctggttggggatgggctcgcggcctatcagcttgttggtggggtgatggcctaccaaggcggtgacgggtagccggcctgagagggtggacggccacactgggactgagatacggcccagactcctacgggaggcagcagtgggggattttgcacaatgggcgcaagcctgatgcagcgacgccgcgtgagggatgacggccttcgggttgtaaacctctttcgctaggttggaagacgctttttggagttgttgactttaactggtaaagaagtaccggctaactacgtgccagcagccgcggtaatacgtagggtactagcgttgtccggaattattgggcgtaaagggcttgtaggtggtttgtcgcgtctgtcgtgaaaacccggggcttaactccgggcttgcggtgggtacaggcaggcttgagtgcggtaggggagactggaattcctggtgtagcggtggaatgcgcagatatcaggaggaataccggtggcgaaggcgggtctctgggccgttactgacactgaggagcgaaagcgtggggagcgaacaggattagataccctggtagtccacgctgtaaacgttgggcactagttgtgggggcttttgttggtttccgcgacgtagctaacgcattaagtgccccgcctggggagtacggccgcaaggctaaaactcaaaggaattgacggggacccgcacaagcggcggagcatgcggattaattcgatgcaacgcgaagaaccttaccaaggcttgacatgcaccggacggctctagagatagggtttccctttgtggctggtgtgcaggtggtgcatggttgtcgtcagctcgtgtcgtgagatgttgggttaagtcccgcaacgagcgcaacccttgtctagtgttgccagcaagtagtgttggggactctctagtgactgccggggttaactcggaggaaggtggggatgacgtcaaatcatcatgccccttatgtcttgggcttcacgcatgctacaatgggcggtacagtgggttgcaataccgtgaggtggagctaatccccaaagccgttctcagttcggatcgtagtctgcaactcgactacgtgaaggtggagtcgctagtaatcgcagatcagcattgctgcggtgaatacgttctcgggtcttgtacacaccgcccgtcacgtcacgaaagttggtaacacccgaagctcatggcctaaccgcgtttgtggggggagtggtcgaaggtgggattggcgattgggacgaagtcgtagcaaggtagctgtaccggaaggtgcggctggaacacctcctt

SEQ ID NO: 2 - DNA, 1479 bp, Actinomyces sp.
  source 1..1479: mol_type = genomic DNA
gacgaacgctggcggcgtgcttaacacatgcaagtcgaacgggatccaagtgggcttgcttgcttggtgagagtggcgaacgggtgagtaacacgtgagtaacctgcccccttctttgggataacgaccagaaatggttgctaatactgaatattcacgcattgtcgcatggtggtgtgtggaaaggtttttctggtgggggatgggctcgcggcctatcagcttgttggtggggtgatggcctaccaaggctttgacgggtagccggcctgagagggtgaccggtcacactgggactgagatacggcccagactcctacgggaggcagcagtggggaatattgcacaatgggcgaaagcctgatgcagcgacgccgcgtgagggatgacggccttcgggttgtaaacctctttcagtaaggacgcaggcctaggcttttgtttgggttgactttacttacagaagaagcgccggctaactacgtgccagcagccgcggtaatacgaagggcgcaagcgttgtccggaattattgggcgtaaagagctcgtaggcggtttgtcgcgtctgtcgtgaaaacccacagcttaactgtgggcttgcggtgggtacgggcaggcttgagtgcggtaggggagactggaattcctggtgtagcggtggaatgcgcagatatcaggaggaacaccggtggcgaaggcgggtctctgggccgttactgacgctgaggagcgaaagcatggggagcgaacaggattagataccctggtagtccatgccgtaaacgttgggcactaggtgtgggggctgtttccatggttcctgcgccgtagctaacgcattaagtgccccgcctggggagtacggtcccaaggctaaaactcaaaggaattgacgggggcccgcacaagcggcggagcatgcggattaattcgatgcaacgcgaagaaccttaccaaggcttgacatacatccagagtctgcagagatgtgggcgcatttagttgggggtgtacaggtggtgcatggttgtcgtcagctcgtgtcgtgagatgttgggttaagtcccgcaacgagcgcaacccttgtctcatgttgccagcacgttatggtggggactcgtgagagactgccggggttaactcggaggaaggtggggatgacgtcaaatcatcatgccccttatgtcttgggcttcacgcatgctacaatggccagtacagagggttgcgatatcgtaaggttgagcgaatcccttaaagctggtctcagttcggattggggtctgcaactcgaccccatgaagtcggagtcgctagtaatcgcagatcagcaacgctgcggtgaatacgttctcgggccttgtacacaccgcccgtcacgtcacgaaagttggtaacacccgaagcccatggcctaaccgtttggggggagtggtcgaaggtgggactggcgattgggacgaagtcgtaacaaggt

SEQ ID NO: 3 - DNA, 1493 bp, Anaerovorax sp.
  source 1..1493: mol_type = genomic DNA
gaggaacgctggcggcgtgcctaacacatgcaagtcgagcgagaaatagtcagacggagatttcggttgaaggatggttatggaaagcggcggacgggtgagtaacgcgtaggcaacctgcctcatgcagagggatagcctcgggaaaccgggattaaaacctcataaggccaaggggacacatgttttcgtggtcaaagatttatcggcatgagatgggcctgcgtctgattagctggttggtgaggtaacggctcaccaaggcgacgatcagtagccgacctgagagggtaaacggccacattggaactgagacacggtccaaactcctacgggaggcagcagtggggaatattgcacaatgggcgaaagcctgatgcagcaacgccgcgtgagcgaagaaggcctttgggtcgtaaagctctgtcgatagggaagaaaaaggtgacggtacctatcaagaaagccccggctaactacgtgccagcagccgcggtaatacgtagggggcgagcgttatccggaattattgggcgtaaagagtgcgtaggtggctagataagcgtggggtgaaaggcagtggcttaaccattgtaagccctgcgaactgtgtagcttgagtgcaggaggggaaagtggaattcctagtgtagcggtgaaatgcgtagatattaggaggaacaccggtggcgaaggcggctttctggactgtaactgacactgaggcacgaaagcgtgggtagcaaacaggattagataccctggtagtccacgccgtaaacgatgagcactaggtgttggtctcgcaagagatcggtgccgcagttaacgcagtaagtgctccgcctggggagtacgcacgcaagtgtaaaactcaaaggaattgacggggacccgcacaagcagcggagcatgtggtttaattcgaagcaacgcgaagaaccttaccaggacttgacatctgcctgacattcctttaataggggagttcttttagacaggcaagacaggtggtgcatggttgtcgtcagctcgtgtcgtgagatgttgggttaagtcccgcaacgagcgcaacccttgtcaatagttgccagcagtaagatggggactctattgagactgccgtggataacacggaggaaggtggggatgacgtcaaatcatcatgccccttatgttctgggctacacacgtgctacaatggccggtacagagagaagcgagatagagatatggagcaaacctcagaaaaccggtcccagttcggattgtaggctgcaactcgcctacatgaagttggagttgctagtaatcgcagatcagaatgctgcggtgaatgcgttcccgggtcttgtacacaccgcccgtcacaccatggaagttgggggtgcccgaagtcggttaggaaataggctgccgaaggcaaaaccaatgactggggtgaagtcgtaacaaggtagccgtatcggaaggtgcggctggatcacctcctt

SEQ ID NO: 4 - DNA, 1502 bp, Bacteroides sp.
  source 1..1502: mol_type = genomic DNA
gatgaacgctagctacaggcttaacacatgcaagtcgatgggcatcatggtattagcttgctaatactgatggcgaccggcgcacgggtgagtaacacgtatccaacctggctttcactcagggatagcccatcgaaagatggattaatacttgatggtatatacatttcgcatgttttgtatattaaagatttatcggtgatagatggggatgcgtaccattagtttgttggcggggtaacggcccaccaagacttcgatggttaggggttctgagaggaaggtcccccacattggaactgagacacggtccaaactcctacgggaggcagcagtgaggaatattggtcaatggacgtaagtctgaaccagccaagtagcgtgaaggatgactgccctctgggttgtaaacttcttttatatgtgaataaattttcctacgtgtaggtttttgcatgtaacatatgaataagcatcggctaactccgtgccagcagccgcggtaatacggaggatgcgagcgttatccggatttattgggtttaaagggagcgtaggtgggttattaagtcagcgttgtgaaagtttgcggctcaaccgtaaaattgccgttgatactgatagtcttgagttcagtagaggtgtgcggaattcgtggtgtagcggtgaaatgcttagatatcacgaagaactccaattgcgtaggcagctcactatactgttactgacactgatgctcgaaagtgtgggtatcaaacaggattagataccctggtagtccacacagtaaacgatgaatactcgctgtttgcgatatagtgtaagcggccaagcgaaagcgttaagtattccacctggggagtacgccggcaacggtgaaactcaaaggaattgacgggggcccgcacaagcggaggaacatgtggtttaattcgatgatacgcgaggaaccttacctgggcttaaatattagatgcataacatagagatatgtttttcttcggacttctattaaggtgctgcatggttgtcgtcagctcgtgccgtgaggtgtcggcttaagtgccataacgagcgcaaccctaatgtttagttactaacagtttatgctgaggactctagacagactgccgtcgtaagaagtgaggaaggttaggatgacgtcaaatcagcacggcccttacgtccagggctacacacgtgttacaatggggggtacagaaggttgctacacggcgacgtgatgctaatcccaaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgtaaggagcgtcctagggtaaatctggtgattggggctaagtcgtaacaaggtagccgtaccggaaggtgcggctggatcacctcctt

SEQ ID NO: 5 - DNA, 1465 bp, Bergeyella zoohelcum
  source 1..1465: mol_type = genomic DNA
gatgaacgctagcgggaggcctaacacatgcaagccgagcgggatttgttggttagcttgctaactgataatgagagcggcgtacgggtgcgtaacacgtgtgcaacctgcctttatctgggggatagcctttcgaaaggaagattaataccccataatatattgattggcatcgattaatattgaaagctgaggcggatagagatgggcacgcgtaagattagctagttggtgaggtaacggctcaccaaggctgcgatctttagggggcctgagagggtgatcccccacactggtactgagacacggaccagactcctacgggaggcagcagtgaggaatattggacaatgggtgagagcctgatccagccaccccgcgtgaaggactaaggacctatggtttgtaaacttcttttatacagggataaacctactctcgtgagggtagctgaaggtactgtatgaataagcaccggctaactccgtgccagcagccgcggtaatacggagggtgcaagcgttatccggatttattgggtttaaagggtccgtaggcgggtcgataagtcagtggtgaaagcctgcagcttaactgtagaactgccgttgatactgtcggtcttgagtgtatttgaggtagctggaatgagtagtgtagcggtgaaatgcatagatattactcagaacaccaattgcgaaggcaggttaccaagttacaactgacgctgatggacgaaagcgtggggagcgaacaggattagataccctggtagtccacgctgtaaacgatgctaactcgtttttggggtattgtacttcagagaccaagcgaaagtgataagttagccacctggggagtacgaacgcaagtttgaaactcaaaggaattgacgggggcccgcacaagcggtggattatgtggtttaattcgatgatacgcgaggaaccttaccaagacttaaatgggaattgacagctgtagaaatacggttttcttcggacaattttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgtcttgggccacacacgtaatacaatggccggtacagagggcagctacactgcgaagtgatgcgaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtcaaaggagctgcctagggtaaaacaggtaactagggctaagtcgtaacaaggt

SEQ ID NO: 6 - DNA, 1455 bp, Capnocytophaga sp.
  source 1..1455: mol_type = genomic DNA
gatgaacgctagcggcaggcctaacacatgcaagtcgaggggtagagctactttagtagtttgagaccggcgcacgggtgcgtaacgcgtatgcaatctaccttatacagagggatagcccagagaaatttggattaatacctcatagtatatttgagtggcatcatttagatattaaagatttatcggtataagatgagcatgcgtcccattagttagttggtgtggtaacggcataccaagacgatgatgggtaggggtcctgagagggagatcccccacactggtactgagacacggaccagactcctacgggaggcagcagtgaggaatattggtcaatggaggcaactctgaaccagccatgccgcgtgcaggaagaatgtcctatggattgtaaactgcttttgtacgggaagaaaacgtgctatgtatagcaaactgacggtaccgtaagaataaggatcggctaactccgtgccagcagccgcggtaatacggaggatccgagcgttatccggaatcattgggtttaaagggtccgtaggcgggattataagtcagtggtgaaagtttgcagcttaactgtaaaattgccattgatactgtagttcttgaatttttgtgaagtaactagaatatgtagtgtagcggtgaaatgcatagatattacatggaataccaattgcgaaggcaggttactaacaaaggattgacgctgatggacgaaagcgtgggtagcgaacaggattagataccctggtagtccacgctgtaaacgatggatactagctgtttggttttcggactgagtggctaagcgaaagtgataagtatcccacctggggagtacgcacgcaagtgtgaaactcaaaggaattgacgggggcccgcacaagcggtggagcatgtggtttaattcgatgatacgcgaggaaccttaccagggcttaaatgtagattgacttatttggaaacagatatttcttcggacaatttacaaggtgctgcatggttgtcgtcagctcgtgccgtgaggtgtcaggttaagtcctataacgagcgcaacccctgttgttagttgccaacgagtgatgtcgggaactctagcaagactgccggtgtaaaccgtgaggaaggtggggatgacgtcaaatcatcacggcccttacgtcctgggccacacacgtgctacaatggccggtacagagagcagccacttggtgacaaggagcgaatctataaagccggtcacagttcggattggagtctgcaactcgactctatgaagctggaatcgctagtaatcggatatcagccatgatccggtgaatacgttcccgggccttgtacacaccgcccgtcaagccatggaagctgggggtacctgaagtcggtgaccgcaaggagctgcctagggtaaaactggtaactggggctaagtcgtaacaaggt

SEQ ID NO: 7 - DNA, 1500 bp, unidentified
  source 1..1500: mol_type = unassigned DNA; note = Clostridiales sp.
gatgaacgctggcggcgtgcttaacacatgcaagtcgaacggaacgtaaccgaggaaatttttcgaagtggaatcagttacgtttagtggcggacgggtgcgtaacacgtgggcaacctgccctgcttttcggaataacgccgggaaaccgtcgctaatgccgaatgatatgaggagaaggcatcttcttttcatcaaagaatttcgaagcaggatgggcccgcgccggattagctggttggtgaggtaacggctcaccaaggcgacgatccgtagccgacctgagagggtgatcggccacactggaactgagacacggtccagactcctacgggaggcagcagtggggaatattgcacaatgggcgaaagcctgatgcagcaacgccgcgtgagcgatgaaggctttcgagtcgtaaagctctgtcgcaggggaagaaaaagacggtaccctgtgagaaagccccggctaactacgtgccagcagccgcggtaatacgtagggggcaagcgttatccggaattactgggcgtaaagggtgcgtaggcggcttttcaagtcaaggcttaaaggctaacggctcaaccgtagtaaggccttgaaactggagagcttgagtatcggagaggaaagtggaattcccagtgtagcggtgaaatgcgtagatattgggaggaacaccagtggcgaaggcgactttctggacgatgactgacgctgaggcacgaaagcgtggggagcaaacaggattagataccctggtagtccacgccgtaaacgatgagtactaggtgtcaggtgtcaaagcttggtgccgcagttaacacagtaagtactccgcctggggagtacgctcgcaagagtgaaactcaaaggaattgacggggacccgcacaagtagcggagcatgtggtttaattcgaagcaacgcgaagaaccttacctaagcttgacatcccgttgaccggggcttaatcgcccctttcccttcggggacagcggagacaggtggtgcatggttgtcgtcagctcgtgtcgtgagatgttgggttaagtcccgcaacgagcgcaacccttgtttttagttgccagcggtttggccgggcactctaaaaagactgccgaggataactcggaggaaggtggggatgacgtcaaatcatcatgccccttatgcttagggctacacacgtgctacaatggctgttacaaagggccgcgaggccgcaaggcggagccaatcccataaaaacagtctcagttcggattgcaggctgaaactcgcctgcatgaagtcggagttactagtaatcgcgaatcagcatgtcgcggtgaatgcgttcccgggtcttgtacacaccgcccgtcacaccatggaagcttggggcacccaaagtccgtgatccaacgaaagaggaagcggcctaaggtgaaacaagtgactggggtgaagtcgtaacaaggtagccgtatcggaaggtgcggctggatcacctcctt

SEQ ID NO: 8 - DNA, 1500 bp, unidentified
  source 1..1500: mol_type = unassigned DNA; note = Clostridiales sp.
gacgaacgctggcggcgcgcctaacacatgcaagtcgagcgggagtgattgaggggtaacccgagagcactttagcggcggacgggtgagtaacgcgtgagcaacctgccttttacagggggataacgtgccgaaaggcacactaataccgcatgagaccacaagcatacatatggaaggggtcaaaggagaaatccggtaagagatgggctcgcgtccgattagatagttggtgaggtaacggctcaccaagtcaacgatcggtagccgacctgagagggtgatcggccacattggaactgagagacggtccaaactcctacgggaggcagcagtggggaatattgggcaatgggggaaaccctgacccagcgacgccgcgtgaaggaagaaggtcttcggattgtaaacttctgttcttggtgaagagggaaagacggtagccaaggaggaagccccggctaactacgtgccagcagccgcggtaatacgtagggggcgagcgttgtccggaatgattgggcgtaaagggcgcgtaggcggctgtgtaagtctggagtgaaagtcctattttcaaggtaggaattgctttggaaactgcatagcttgagtgcaggagaggttatcggaattcccggtgtagcggtgaaatgcatagagatcgggaggaacaccagtggcgaaggcgggtaactggactgcaactgacgttgaggcgcgaaagtgtggggagcaaacaggattagataccctggtagtccacactgtaaacgatgaatgctaggtgtaggtggtatcgaccccatctgtgccgtcgtaaacacaataagcattccgcctggggagtacggtcgcaagattaaaactcaaaggaattgacgggggcccgcacaagcagcggagcatgtggtttaattcgacgcaacgcgaagaaccttaccaggtcttgacatccgctttaggcatgcagagatgcatgtgggacttcggtccgaagcgagacaggtggtgcatggttgtcgtcagctcgtgtcgtgagatgttgggttaagtcccgcaacgagcgcaacccttgccaatagttactaacggataaagctgaggactctattgggactgccgtggacaacacggaggaaggtggggacgacgtcaaatcatcatgccccttatgacctgggctacacacgtgctacaatggtcgcaacaaagaggagcgaagccgtaaggtggagcagatctcaaaaaagcgatcccagttcagattgtgggctgcaacccgcccacatgaagtcggagttgctagtaatcgcgaatcagcatgtcgcggtgaatacgttcccgggccttgtacacaccgcccgtcacaccatgggagttgatagcgcccgaaggcagagaggtaaccgcaaggggccatctgacgaaggcgagatcgatgactggggtgaagtcgtaacaaggtagccgtatcggaaggtgcggctggatcacctcctt

SEQ ID NO: 9 - DNA, 1524 bp, Desulfomicrobium orale
  source 1..1524: mol_type = genomic DNA
aatgaacgctggcggcgtgcctaacacatgcaagtcgaacgagaaagttcctccgggaatgagtagagtggcgcacgggtgagtaacgcgtgggtaatctgcccttggatttgggataacttcgcgaaagtggagctaataccggatagtctggctttgtaaaaggagtcggtaaaggatgcctctgcatatgcattcgtccgaggatgagcccgcgtctcattagctagttggtagggtaacggcctaccaaggcgacgatgagtagctggtctgagaggatgatcagccacactgggactgaaacacggcccagactcctgcgggaggcagcagtggggaatattgcgcaatgggcgaaagcctgtcgcagcaacgccgtgtgagggatgaaggttttcggatcgtaaacctctgtctgaagggaagaagtgatgcgggtccaataggcccgcattttgacggtaccttcaaaggaagcaccggctaactccgtgccagcagccgcggtaatacggagggtgcaagcgttattcggaattactgggcgtaaagcgcacgtaggcggccttgtaagtcaggtgtgaaatcccacggctcaaccgtggaactgcacttgaaactgcggggctcgaatcctggagagggcggcggaattcctggtgtaggagtgaaatccgtagatatcaggaggaacaccggtggcgaaggcggccgcctggacaggtattgacgctgaggtgcgaaagtgtggggagcaaacaggattagataccctggtagtccacaccgtaaacgatggatactaggtgtcggggacttgatcctcggtgccgcagttaacgcgttaagtatcccgcctggggagtacggtcgcaaggctgaaactcaaagaaattgacaggggcccgcataagcggtggagtatgtggtttaattcgatgcaacgcgaagaaccttacctgggcttgacatcctgggaattccgcagagatgcggaagtgcccttcggggaatccagagacaggtgctgcatggctgtcgtcagctcgtgccgtgaggtgttgggttaagtcccgcaacgagcgcaacccctatcctcagttgccagcaggtaaagctgggcactctgtggagactgcccgggttaaccgggaggaaggtggggacgacgtcaagtcatcatggcccttacgcccagggctacacacgtactacaatggtgggcacaaagggcagcgaaaccgcaaggtccagccaatcccaaaaaacccatcatagtccggattgcagtctgcaactcgactgcaagaagttggaatcgctagtaatcccggatcagcatgccggggtgaatacgttcccgggccttgtacacaccgcccgtcacaccacgaaagtcggttctacccgaagccgccaggctaacccgcaagggatgcaggcgtctacggtagggctggtaattggggtgaagtcgtaacaaggtagccgtaggggaacctgcggctggatcacctcctt

SEQ ID NO: 10 - DNA, 1500 bp, Filifactor sp.
  source 1..1500: mol_type = genomic DNA
gatgaacgctggcggcgtgcttaacacatgcaagtcgaacggacgaatcttatcggagtttttcggaacaaagaaaagataagttagtgacggacgggtgcgtaacgtgtgggtaatctgcctttgtcatgggaataactgcttgaaagagcagctaataccgaataacatatgcaataggcatctattggatatcaaagagaaatcggacaaagatgagcccgcatctgattagctagttggtagggtaacagcctaccagggcgacgatcagtagccgacctgagagggtgaacggccacattggaactgagacacggtccaaactcctacgggaggcagcagtggggaatattgcacaatggggggaaccctgatgcagcaacgccgcgtgagtgaagaaggctttcgagtcgtaaaactctgttgtaagggaagataatgacagtaccttaaaagaaagccccggctaactacgtgccagcagccgcggtaatacgtagggggcaagcgttatccggaatcactgggcgtaaagggtgcgcaggtggtctgtcaagttagaggtgaaaggcataggctcaaccaatgtaagcctttaaaactgacggacttgagtgcaggagaggaaagtggaattcctagtgtagcggtgaaatgcgtagatattaggaggaataccggtggcgaaggcgactttctggactgtaactgacactgaggcacgaaagcgtggggagcaaacaggattagataccctggtagtccacgccgtaaacgatgagtactaggtgtcgggggcgacctcggtgccgcagttaacacattaagtactccgcctggggagtacgctcgcaagagtaaaactcaaaggaattgacggggacccgcacaagcagcggagcatgtggtttaattcgaagcaacgcgaagaaccttacctaaacttgacataccgatgacgattcggtaatgcgaatattcctttcggggacatcggatacaggtggtgcatggttgtcgtcagctcgtgtcgtgagatgttgggttaagtcccgcaacgagcgcaacccttgtcaatagttgccagcacttagggtggggactctattgagacagctaaggacaacttagaggaaggtggggatgacgtcaaatcatcatgccccttatgtttagggctacacacgtgctacaatggtcattacagagggaggcgaagctgtgaagcggagcgaagcccaaaaagatgatctaagttcggattgtaggctgaaactcgcctacatgaagttggagttgctagtaatcgcgaatcagcatgtcgcggtgaatgcgttcccgggtcttgtacgcaccgcccgtcacaccatgggagttcggggggcccaaagtcagtgagcaaaccgagagggtgcagctgcctaaggcaaaacgaatgactggggtgaagtcgtaacaaggtagccgtatcggaaggtgcggctggatcacctcctt

SEQ ID NO: 11 - DNA, 1472 bp, unidentified
  source 1..1472: mol_type = unassigned DNA
gatgaacgctggcggcgtgcttaacacatgcaagtcgagcggagtttacaatggaaagatcttcggaaatggaagctgtaaacttagcggcggacgggtgcgtaacgcgtgggtaatctgcccttatcatcgggataacacaacgaaagatgtgctaataccgaataacatgcattttcggcatcgaagatgtatcaaagtgttagcggataaggatgagcctgcgtctgattagcttgttggtggggtaatggcctaccaaggcgacgatcagtagccgacctgagagggtgatcggccacactggaactgagacacggtccagactcctgcgggaggcagcagtggggaatattgcacaatgggcgcaagcctgatgcagcaacgccgcgtgagcgatgaaggtcttcggatcgtaaagctctgtcgcatggggagataatgacggtaccatgtgaggaagccccggctaactacgtgccagcagccgcggtaatacgtagggggcaagcgttatccggaattactgggcgtaaagggtgcgtaggtggcctgacaagtcaggggtcaaaggcaacggctcaaccgttgtaagcctttgaaactgtcgggcttgagttctggaggggaaatcggaattcctagtgtagcggtgaaatgcgtagatattaggaggaacaccggtggcgaaggcggatttctggacagatactgacactgaggcacgaaagcgtggggagcaaacaggattagataccctggtagtccacgccgtaaacgatgagtactaggtgttggcccgttagggtcggtgccgcagttaacacattaagtactccgcctgggaagtacgctcgcaagagtgaaactcaaaggaattgacggggacccgcacaagtagcggagcatgtggtttaattcgaagcaacgcgaagaaccttacctaagcttgacatccctttgaccgcagggtaatgcctgctttcccttcggggacagaggagacaggtggtgcatggttgtcgtcagctcgtgtcgtgagatgttgggttaagtcccgcaacgagcgcaacccttgtctttagttgccatcaggttatgctgggcactctagagagactgccgaggacaactcggaggaaggtggggatgacgtcaaatcatcatgccccttatgcttagggctacacacgtgctacaatggctgttacagagggtagccaaaccgtgaggtggagccaatcccataaaaacagtctaagttcggattgtaggctgaaactcgcctacatgaagctggagttactagtaatcgcagatcagcatgctgcggtgaatgcgttcccgggtcttgtacacaccgcccgtcacaccatgggagctgggggggcccaaagtcaactatctaactgcaaagaggaagttgcctaaggcaaaaccagtgactggggtgaagtcgtaacaaggtagcc

SEQ ID NO: 12 - DNA, 1456 bp, Fusobacterium sp.
  source 1..1456: mol_type = genomic DNA
gatgaacgctgacagaatgcttaacacatgcaagtcaacttgaacgtgggtttttaacttaggtttgggtggcggacgggtgagtaacgcgtaaggaacttgcctcacagatagggacaacatttagaaatgaatgctaatacctaatattatgaaaatagggcatcctataattatgaaagctaaatgcgctgtgagagagctttgcgtcccattagctagttggagaggtaacggctcaccaaggcgatgatgggtagccggcctgagagggtgaacggccacaagaggactgagacacggcccttactcctacgggaggcagcagtggggaatattggacaatggaccaagagtctgatccagcaattctgtgtgcacgatgaagtttttcggaatgtaaagtgctttcagttgggaagaaaagaatgacggtaccgacagaagaagtgacggctaaatacgtgccagcagccgcggtaatacgtatgtcacgagcgttatccggatttattgggcgtaaagtgcgtctaggtggttatgtaagtctgatgtgaaaatgcagggctcaactctgtattgcgttggaaactgtgtaactagagtactggagaggtaagcggaactacaagtgtagaggtgaaattcgtagatatttgtaggaatgccgatggggaagccagcttactggacagatactgacgctaaagcgcgaaagcgtgggtagcaaacaggattagataccctggtagtccacgccgtaaacgatgattactaggtgttgggggtcgaacctcagcgcccaagcaaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcttaggaatgagatagagatatttcagtgtcccttcggggaaacctaaagacaggtggtgcatggctgtcgtcagctcgtgtcgtgagatgttgggttaagtcccgcaacgagcgcaacccttttcgtatgttaccatcattaagttggggactcatgcgatactgcctgcgatgagcaggaggaaggtggggatgacgtcaagtcatcatgccccttatacgctgggctacacacgtgctacaatgggtagaacagagagttgcaaagccgtgaggtggagctaatctcagaaaactattcttagttcggattgtactctgcaactcgagtacatgaagttggaatcgctagtaatcgcgaatcagcaatgtcgcggtgaatacgttctcgggtcttgtacacaccgcccgtcacaccacgagagttggttgcacctgaagtagcaggcctaaccgtaaggagggatgctccgagggtgtgattagcgattggggtgaagtcgtaacaaggt

SEQ ID NO: 13 - DNA, 1443 bp, Fusobacterium sp.
  source 1..1443: mol_type = genomic DNA
gatgaacgctgacagaatgcttaacacatgcaagtcaacttgaacttcggtttgggtggcggacgggtgagtaacgcgtaaagaacttgcctcacagtctaggacaacatctggaaacggatgctaaaactggatattatgaactatgcgcatgcatagattatgaaagctatatgcgctgtgagagagttttgcgtcccattagctagttggagaggtaacggctcaccaaggcgatgatgggtagccggcctgagagggcgaacggccacaaggggactgagacacggcccttactcctacgggaggcagcagtggggaatattggacaatggaccaagagtctgatccagcaattctgtgtgcacgatgaagtttttcggaatgtaaagtgctttcagttgggaagaagaaagtgacggtaccaacagaagaagcgacggctaaatacgtgccagcagccgcggtaatacgtatgtcgcaagcgttatccggatttattgggcgtaaagcgcgtctaggcggctaaataagtctgatgtgaaaatgcggggctcaactccgtattgcgttggaaactgtatagctagggtactggagaggtaagcggaactacaagtgtagaggtgaaattcgtagatatttgtaggaatgccgatggggaagccagcttactggacagatactgacgctaaagcgcgaaagcgtgggtagcaaacaggattagataccctggtagtccacgccgtaaacgatgattactaggtgttgggggtcgaacctcagcgcccaagcaaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcctactaagtactcagagatgagtatgtgctctttcgggagaaagtagagacaggtggtgcatggctgtcgtcagctcgtgtcgtgagatgttgggttaagtcccgcaacgagcgcaacccctttcgtatgttaccatcattaagttggggactcatgcgatactgcctgcgatgagcaggaggaaggtggggatgacgtcaagtcatcatgccccttatacgctgggctacacacgtgctacaatgggtagaacagagagcagcgaagctgtgaagtggagcaaatctcagaaaactattcttagttcggattgtactctgcaactcgagtacatgaagttggaatcgctagtaatcgcaaatcagcaatgttgcggtgaatacgttctcgggtcttgtacacaccgcccgtcacaccacgagagttggttgcacctgaagtaacaggcctaaccgtaaggagggatgttccgagggtgtgattagcgattggggtgaagtcgtaacaaggt

SEQ ID NO: 14 - DNA, 1528 bp, Gemella palaticanis
  source 1..1528: mol_type = genomic DNA
gacgaacgctggcggcgtgcctaatacatgcaagtcgagcggagttgaataagtgcttgcactaattcaacttagcggcgaacgggtgagtaacacgtaaagaacctgcctcatagactgggacaactactggaaacggtagctaataccggataacatatttttctgcatggaagaatattgaaagtcggtcatgctgacactatgagatggccttgcggtgcattagctagttggtagggtaaaggcctaccaaggcgacgatgcatagccgacctgagagggtgatcggccacactgggactgagacacggcccagactcctacgggaggcagcagtagggaatcttccgcaatggacgaaagtctgacggagcaacgccgcgtgagtgaagaaggatttcggttcgtaaaactctgttgttagggaagaatgattgtatagtaactatatacagtagagacggtacctaaccagaaagccacggctaactacgtgccagcagccgcggtaatacgtaggtggcaagcgttgtccggaattattgggcgtaaagcgcgcgcaggtggtttaaaaagtctgatgtgaaagcccacggctcaaccgtggaaggtcattggaaactgttaaacttgagtgcaggagagaaaagtggaattcctagtgtagcggtgaaatgcgtagagattaggaggaacaccagtggcgaaggcggctttttggcctgtaactgacactgaggcgcgaaagcgtggggagcaaacaggattagataccctggtagtccacgccgtaaacgatgagtgctaagtgttggagtcataagacttcagtgctgcagctaacgcattaagcactccgcctggggagtacggtcgcaagactgaaactcaaaggaattgacggggacccgcacaagcggtggagtatgtggtttaattcgaagcaacgcgaagaaccttaccaagtcttgacatacagtgaagatttaagagattagattgtttatacctatggtataacactgatacaggtggtgcatggttgtcgtcagctcgtgtcgtgagatgttgggttaagtcccgcaacgagcgcaacccttatatctagttgccagcagtaagatggggactctagatagactgccagtgataaactggaggaaggtggggatgacgtcaaatcatcatgccccttatgacttgggctacacacgtactacaatggataggaacaaagagaagcaacctcgtaagagcaagccaacctcataaaactattctcagttcggattgtagtctgcaactcgactacatgaagctggaatcgctagtaatcgcgaatcagaatgtcgcggtgaatacgttcccgggtcttgtacacaccgcccgtcacaccacgggagtttgtaacacccgaagacggtggcctaaccgcaaggagggagccggtcacggtgggatagatgattggggtgaagtcgtaacaaggtagccgtatcggaaggtgcggctggatcacctcctt

SEQ ID NO: 15 - DNA, 1524 bp, Granulicatella sp.
  source 1..1524: mol_type = genomic DNA
gacgaacgctggcggcgtgcctaatacatgcaagtcgaacgaacggagaaagaagcttgcttttttcaaagttagtggcgaacgggtgagtaacacgtgggtaacctgcccatcagagggggataacattcggaaacggatgctaaaaccgcataggttccttagttgcatgactgaggaaggaaaagaggcgtaagcttctgctggtggatggacccgcggtgcattagctagttggtgaggtaacggctcaccaaggcgatgatgcatagccgacctgagagggtgatcggccacattgggactgagacacggcccaaactcctacgggaggcagcagtagggaatcttccgcaatggacgcaagtctgacggagcaacgccgcgtgagtgaagaaggttttcggatcgtaaaactctgttgttagagaagaataagcataatagtaactgattatgccgtgacggtatctaaccagaaagccacggctaactacgtgccagcagccgcggtaatacgtaggtggcaagcgttgtccggatttattgggcgtaaagcgagcgcaggcggttccttaagtctgatgtgaaagcccacggctcaaccgtggagggtcattggaaactggggaacttgagtgcagaagaggagagtggaattccatgtgtagcggtgaaatgcgtagatatatggaggaacaccagtggcgaaggcgactctctggtctgtaactgacgctgaggctcgaaagcgtgggtagcaaacaggattagataccctggtagtccacgccgtaaacgatgagtgctaagtgttggagggtttccgcccttcagtgctgcagttaacgcattaagcactccgcctggggagtacgaccgcaaggttgaaactcaaaggaattgacggggacccgcacaagcggtggagcatgtggtttaattcgaagcaacgcgaagaaccttaccaagtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcattcagttgggcactctagtgagactgccggtgacaaaccggaggaaggtggggatgacgtcaaatcatcatgccccttatgacttgggctacacacgtgctacaatggatggtacaacgagcagcgaactcgcgagggtaagcgaatctcttaaagccattctcagttcggattgtaggctgcaactcgcctacatgaagccggaatcgctagtaatcgcggatcagcacgccgcggtgaatacgttcccgggtcttgtacacaccgcccgtcacaccacgagagtttgtaacacccaaagtcggtgaggtaacctattggagccagccgcctaaggtgggatagatgattggggtgaagtcgtaacaaggtagccgtatcggaaggtgcggctggatcacctcctt

SEQ ID NO: 16 - DNA, 1472 bp, Helcococcus sp.
  source 1..1472: mol_type = genomic DNA
gacaaacgctggcggcgtgcataacacatgcaagtcgaacgaaatctacatgatttagtggcgaacgggtgagtaacacgtgagaaacctgccttacacagagggatagccgttggaaacgacgattaatacctcatgacaccgtgacatcgcatgatgatgcggtcaaagatttatcggtgtaagatggtctcgcgcctgattagctggttggtggggtaacggcctaccaaggcgacgatcagtagccgatctgagagggtggacggccacattgggactgagacacggcccaaactcctacgggaggcagcagtgggggattttgcacaatggggggaaccctgatgcagcgacgccgcgtgaacgaagaaggccttcgggttgtaaagttctgtccttggtgaagataatgacagtaaccaagaagaaagccccggctaaatacgtgccagcagccgcggtaatacgtatggggcgagcgttgtccggaattattgggcgtaaagggtacgtaggcggttatttaagtccatattaaaaggcattggctcaaccaatgtaaggtgtggaaactggataacttgagtagatgaggggaaagtggaattccaagtgtagcggtgaaatgcgtagatatttggaggaataccggtggcgaaggcgactttctggaatctaactgacgctgaggtacgaaagcgtggggagcaaacaggattagataccctggtagtccacgcagtaaacgatgagtgctaggtgtcgggggtcaaacctcggcgccgccgctaacgcattaagcactccgcctggggagtacgtacgcaagtatgaaactcaaaggaattgacgcggacccgcacaagcagcggagcatgtggtttaattcgaagcaacgcgaagaaccttaccagggcttgacatatacaggaatgtgctagagatagtacagtcttttcgaagacttgtatacaggtggtgcatggttgtcgtcagctcgtgtcgtgagatgttgggttaagtcccgtaacgagcgcaacccctatctttagttaccatcagtaaggctggggactctagagagactgccggtgataaaccggaggaaggtggggatgacgtcaaatcatcatgccctatatgtcctgggctacacacgtgctacaatggtctgaacaaagtgcagcgaactcgtgagggtaagcgaatcacagaaaacagatctcagttcggattgtaggctgcaactcgcctacatgaagtcggagttgctagtaatcgtgaatcagaatgtcacggtgaatgcgttcccgggtcttgtacacaccgcccgtcacaccatgggagttggcaatacccgaagtcgtcgagctaaccgtaaggaggcagacgccgaaggtagggttaatgactggggtgaagtcgtaacaaggtagccgtatcggaaggtgcggctggatcacctcctt

SEQ ID NO: 17 - DNA, 1504 bp, unidentified
  source 1..1504: mol_type = unassigned DNA; note = Lachnospiraceae sp.
gatgaacgctggcggcgtgcctaacacatgcaagtcgaacgaagcgatgaagcggaagttttcggacggaagcatcattgacttagtggcggacgggtgagtaacgcgtgggtaacctgcctcatacagggggataacagtgggaaacgactgctaataccccataagcgcacggcagcaaaagctgcagggcgaaaagcgaatacggtatgagatgggcccgcgtccgattagccagttggcagggtaagagcctaccaaagcgacgatcggtagccggcttgagagagtgaccggccacattgggactgagacacggcccaaactcctacgggaggcagcagtgggggatattgcacaatggggggaaccctgatgcagcgacgccgcgtgaaggaagaagtatttcggtatgtaaacttctatcagcagggaagaaaatgacggtacctgactaagaagcaccggctaaatacgtgccagcagccgcggtaatacgtatggtgcaagcgttatccggaattactgggtgtaaagggagcgtaggcggataggtaagtcagatgtgaaagaccggggctcaactctggggctgcatttgaaactatctaactagagtacaggagaggtaagcggaattcctagtgtagcggtgaaatgcgtagatattaggaggaacatcggtggcgaaggcggcttactggactgaaactgacgctgaggctcgaaagcgtggggagcaaacaggattagataccctggtagtccacgctgtaaacgatgaatactaggtgttggtgcccatggggcatcggtgccgcagctaacgcaataagtattccacctggggagtacgttcgcaagaatgaaactcaaaggaattgacggggacccgcacaagcggtggagcatgtggtttaattcgaagcaacgcgaagaaccttaccaagtcttgacatccttctgaccggcccgtaacggggcctttccttcgggacagaagagacaggtggtgcatggttgtcgtcagctcgtgtcgtgagatgttgggttaagtcccgcaacgagcgcaacccctatccgaagtagccagcaggtagagctgggcactctgcggagacagccggggacaacccggaggaaggcggggatgacgtcaaatcatcatgccccttatgatttgggctacacacgtgctacaatggcgtatacaaagggaagcgagacggtgacgttaagcaaatctcaaaaaaggcgccccagttcggattgtagtctgcgactcgactacatgaagccggaatcgctagtaatcgcaggtcagcatactgcggtgaatacgttcccgggtcttgtacacaccgcccgtcacaccatgggagtcagcaacgcccgaagtcagtgacccaaccgtaaggagggagctgccgaaggcggggcggatgactggggtgaagtcgtaacaaggtagccgtatcggaaggtgcggctggatcacctcctt

SEQ ID NO: 18 - DNA, 1504 bp, unidentified
  source 1..1504: mol_type = unassigned DNA; note = Lachnospiraceae sp.
gatgaacgctggcggcgtgcctaacacatgcaagtcgaacgaagcatatagaaggaagttttcggatggaattttatatgacttagtggcggacgggtgagtaacgcgtggataatctgccttacacagcgggataacaaatggaaacagttgctaataccgcataagcacacagtatcgcatgatacagggtgaaaagatttatcggtgtaagatgagtccgcgtcccattagctggttggtgaggtaaaagcttaccaaggcgacgatgggtagccggcctgagagggtgaacggccacattgggactgagacacggcccagactcctacgggaggcagcagtgggggatattgcacaatgggcgaaagcctgatgcagcgacgccgcgtgaaggaagaagtatttcggtatgtaaacttctatcagcagggaagaaaatgacggtacctgactaagaagccctggctaaatacgtgccagcagccgcggtaatacgtatggggcaagcgttatccggatttactgggcgtaaagggagcgtaggcggcatggcaagtctgatgtgaaagaccggggcttaactccggggttgcattggaaactgtcaagctagagtgtcggagaggtaagcggaattcctggtgtagtggtgaaatgcgtagatattaggaggaacatcggtggcgaaggcggcttactggacgataactgacgctcaggctcgaaagcgtggggagcaaacaggattagataccttggtagtccacgccgtaaacgatgaatactaggtgttggggagctaagctcttcggtgccgcagcaaacgcaataagtattccgcctggggagtacgttcgcaagaatgaaactcaaaggaattgacggggacccgcacaagcggtggagcatgtggtttaattcgaagcaacgcgaagaaccttaccaggccttgacatcgatccgactaggaagtaatgtttctatctcttcggagcggagaagacaggtggtgcatggttgtcgtcagctcgtgtcgtgagatgttgggttaagtcccgcaacgagcgcaacccttatccttagtaaccagcgagtgaagtcggggactctagggagactgccggggatgacccggaggaaggtggggacgacgtcaaatcatcatgccccttacggtctgggctacacacgtgctacaatgggacatacaaagggaggcgaaggagtgatccggagcaaaccccaaaaagtgttcctcagttcggattgtaggctgcaactcgcctacatgaagccggaatcgctagtaatcgcaaatcagaatgttgcggtgaatacgttcccgggtcttgtacacaccgcccgtcacaccatgggagtcgaaaatgcccgaagtcagtgacccaaccgcaaggagggagctgccgaaggcaggtttggtgactggggtgaagtcgtaacaaggtagccgtatcggaaggtgcggctggatcacctcctt

SEQ ID NO: 19 - DNA, 1449 bp, Leptotrichia sp.
  source 1..1449: mol_type = genomic DNA
gatgaacgctgacagaatgcttaacacatgcaagtcgatggggaagtagtacttgtactatggtaaccacggcggacgggtgagtaacgcgtaaagaacttacctttaggacagggataacagagggaaacttctgataagacttgatataattatgtagttgcatgactgtataatgaaaagggatgcctagagagagctttgcgtcctattagttagttggtgtggtaaaggcataccaaggcgatgataggtagccggcctgagagggtggacggccacaaggggactgagatacggcccttactcttacgggaggcagcagtggggaatattggacaatggaggagactctgatccagcaattctgtgtgcacgaagaaggttttcggattgtaaagtgctttcaacagggaagaagaaagtgacggtacctgtagaagaagcgacggctaaatacgtgccagcagccgcggtaatacgtatgtcgcgagcgttatccggaattattgggcataaagggcatctaggcggcatagcaagtcaggggtgaaaacttgtggctcaaccataagcttgcctttgaaactgtgatgctagagtattggaaaggtgggcggaactacacgagtagaggtgaaattcgtagatatgtgtaggaatgccgatgatgaagatagctcactggacgataactgacgctgaagtgcgaaagctaggggagcgaacaggattagataccctggtagtcctagctgtaaacgatgatcactgggtgtgggcaggaagactgtctgtgccgaagcaaatgcgataagtgatccgcctggggagtacgaccgcaaggttgaaactcaaaggaattgacggggacccgcacaagcggtggagcatgtggtttaattcgacgcaacgcgaggaaccttaccagatcttgacatcctcagaagatagtagaagtactattgtgcctttgggaactgagagacaggtggtgcatggctgtcgacagctcgtgtcgtgagatgttgggttaagtcccgcaacgagcgcaacccttatcgttagttaccatcattaagttggggactctagcgagactgcctgcgaagagcaggaggaaggtggggatgacgtcaagtcatcatgccccttatgatctgggctacacacgtgctacaatgggcagtacaaagaggggcgagatggtgacatggagcaaatcttgaaagctgttctaagttcggattgaagtctgcaactcgacttcatgaagttggaatcgctagtaatcgcaaatcagcaatgttgcggtgaatacgttctcgggtcttgtacacaccgcccgtcacaccacgagagttgtttgcacctgaagttactggtctaaccgtaaggaggaaggtacctaaggtgtgaatagtgattggggtgaagtcgtaacaaggt

SEQ ID NO: 20 - DNA, 1507 bp, Moraxella sp.
  source 1..1507: mol_type = genomic DNA
attgaacgctggcggcaggcttaacacatgcaagtcgaacgatgattactagcttgctagtatgattagtggcgaacgggtgagtaatgcttaggaatctgcctattagtgggggacaacattccgaaaggaatgctaataccgcatacgtcctacgggagaaagcaggggatcttttgaccttgcgctaatagatgagcctaagtcggattagctagttggtggggtaaaggcctaccaaggcgacgatctgtagctggtctgagaggatgatcagccacactgggactgagacacggcccagactcctacgggaggcagcagtggggaatattggacaatgggcgaaagcctgatccagccatgccgcgtgtgtgaagaaggccttttggttgtaaagcactttaagtggggaggaaaagctaataattaatacctattagccctgacgttacccacagaataagcaccggctaactctgtgccagcagccgcggtaatacagagggtgcaagcgttaatcggaattactgggcgtaaagcgagcgtaggtggttacttaagtcagatgtgaaagccccgggcttaacctgggaactgcatctgatactgggtaactagagtaggtgagagggaagtagaattccaggtgtagcggtgaaatgcgtagagacctggaggaataccgatggcgaaggcagcttcctggcatcatactgacactgaggttcgaaagcgtgggtagcaaacaggattagataccctggtagtccacgccgtaaacgatgtctaccagtcgttgggtcttttaaagacttagtgacgcagttaacgcaataagtagaccgcctggggagtacggccgcaaggttaaaactcaaatgaattgacgggggcccgcacaagcggtggagcatgtggtttaattcgatgcaacgcgaagaaccttacctggtcttgacatagtaggaatcctgcagagatgcgggagtgccttcgggaatctacatacaggtgctgcatggctgtcgtcagctcgtgtcgtgagatgttgggttaagtcccgcaacgagcgcaacccttttccttagttaccagcgggttatgccgggaactctaaggatactgccagtgacaaactggaggaaggcggggacgacgtcaagtcatcatggcccttacgaccagggctacacacgtgctacaatggtaggtacaaagggttgctacacagcgatgtgatgctaatctcaaaaagcctatcgtagtccggattggagtctgcaactcgactccatgaagtcggtatcgctagtaatcgcagatcagaatgctgcggtgaatacgttcccgggccttgtacacaccgcccgtcacaccatgggagttgatctcaccagaagtgggtagcctaacgcaagagggcgctcaccacggtggggtcgatgactggggtgaagtcgtaacaaggtagccgtaggggaacctgcggctggatcacctcctt

SEQ ID NO: 21 - DNA, 1501 bp, Moraxella sp.
  source 1..1501: mol_type = genomic DNA
attgaacgctggcggcaggcttaacacatgcaagtcgaacggaagttactagcttgctagtaactttagtggcgaacgggtgagtaatatttaggaatctgcctcataacgggggatagctcggggaaactcgaattaataccgcatacaccctacgggggaaaggtggcgcaagctgccgatatgagatgagcctaaatcagattagctagatggtggggtaaaggcctaccatggcgacgatctgtaactggtctgagaggatgatcagtcacaccggaactgagacacggtccggactcctacgggaggcagcagtggggaatattggacaatgggcgaaagcctgatccagccatgccgcgtgtgtgaagaaggccataaggttgtaaagcactttaagcagggaggaagaacttttagttaatagctaaaagtggtgacgttacctgcagaataagcaccggctaactctgtgccagcagccgcggtaatacagagggtgcgagcgttaatcggaattactgggcgtaaagcgagcgtaggtggttgattaagtcagctgtgaaatccccgggcttaacctgggaaagtcagctgatactggttaactagagtatgtgagaggaaagtagaattccaggtgtagcggtgaaatgcgtagagatctggaggaataccgatggcgaaggcagctttctggcataatactgacactgaggttcgaaagcgtgggtagcaaacaggattagataccctggtagtccacgccgtaaacgatgtctactagccgttggttaccaagagtagcaagtggcgcagcaaacgcgataagtagaccgcctggggagtacggccgcaaggttaaaactcaaatgaattgacgggggcccgcacaagcggtggagcatgtggtttaattcgatgcaacgcgaagaaccttacctggtcttgacatatctagaatcctacagagatgtgggagtgccttcgggaattagaatacaggtgctgcatggctgtcgtcagctcgtgtcgtgagatgttgggttaagtcccgcaacgagcgcaacccttatccttagttaccatcaagtgaagttggggactctaaggagactgccagtgacaaactggaggaaggcggggacgacgtcaagtcatcatggcccttacgaccagggctacacacgtgctacaatggtaggtacagagggtcgctacactgcgaagtgatgccaatctcaaaaagcctatcgtagtccagattggagtctgcaactcgactccatgaagtcggaatcgctagtaatcgcggatcagaatgtcgcggtgaatacgttcccgggccttgtacacaccgcccgtcacaccatgggagtctattgcaccagaagtagttagcctaacgcaagagggcgattaccacggtgtggtcgatgactggggtgaagtcgtaacaaggtagccgtaggggaacctgcggctggatcacctcctt

SEQ ID NO: 22 - DNA, 1513 bp, Neisseria animaloris
  source 1..1513: mol_type = genomic DNA
attgaacgctggcggcatgctttacacatgcaagtcgaacggcagcacggaagagcttgctcttttggtggcgagtggcgaacgggtgagtaacgcatcggaacgtaccgagtagtgggggataactgtccgaaaggatggctaataccgcatattctctgaggaggaaagcaggggaccttcgggccttgcgctatttgagcggccgatgtctgattagctagttggtggggtaaaggcctaccaaggcgacgatcagtagcgggtctgagaggatgatccgccacactgggactgagacacggcccagactcctacgggaggcagcagtggggaattttggacaatggggggaaccctgatccagccatgccgcgtgtctgaagaaggccttcgggttgtaaaggacttttgtcagggaagaaaagcttgaggttaatacccttgagtgatgacggtacctgaagaataagcaccggctaactacgtgccagcagccgcggtaatacgtagggtgcaagcgttaatcggaattactgggcgtaaagcgggcgcagacggttacttaagtcagatgtgaaatccccgggctcaacctgggaactgcgtttgaaactgggtgactagagtatgtcagaggggggtagaattccacgtgtagcagtgaaatgcgtagagatgtggaggaataccgatggcgaaggcagccccctgggataatactgacgttcatgcccgaaagcgtgggtagcaaacgggattagataccctggtagtccacgccctaaacgatgtcaattagctgttggggtacttgatgccttagtagcgtagctaacgcgtgaaattgaccgcctggggagtacggtcgcaagattaaaactcaaaggaattgacggggacccgcacaagcggtggatgatgtggattaattcgatgcaacgcgaagaaccttacctggtcttgacatgtacggaatcctccagagacggaggagtgccttcgggaaccgtaacacaggtgctgcatggctgtcgtcagctcgtgtcgtgagatgttgggttaagtcccgcaacgagcgcaacccttgtcattagttgccatcattaagttgggcactctaatgagactgccggtgacaagccggaggaaggtggggatgacgtcaagtcctcatggcccttatgaccagggcttcacacgtcatacaatggtcggtacagagggtagccaagccgcgaggtggagccaatcccaaaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgtaaggagcccgcttaccacggtatgcttcatgactggggtgaagtcgtaacaaggtagccgtaggggaacctgcggctggatcacctcctt

SEQ ID NO: 23 - DNA, 1498 bp, Odoribacter denticanis
  source 1..1498: mol_type = genomic DNA
gatgaacgctagcgataggcttaacacatgcaagtcgaggggtaacaggcggtagcaatactgtgctgacgaccggcggatgggtgagtaacgcgtatgcaatctaccttttacccggggatagcccatggaaacgtggattaatacccgatgcatttcttttgtggcctcatgaagggaataaagatttatcggtaagagatgagcatgcgttccattaggaagttggtaaggtaacggcttaccaatccgatgatggataggggttctgagaggaaggtcccccacactggaattgagaaacggtccagactcctacgggaggcagcagtgaggaatattggtcaatggtcgtaagactgaaccagccaagtcgcgtgaaggaaactgccctatgggttttcaacttcttttgtcagggaagaataaggaggattcaattctccgatgccggtacctgacgaataaggatcggctaactccgtgccagcagccgcggtaatacggaggatccgagcgttatccggatttattgggtttaaagggtgcttaggcggcaaattaagttagtggttaaatagttcggctcaaccggatttcgccattaaaactgatatgctagagattaaacgaggtaggcggaataagttaagtagcggtgaaatgcatagatataacttagaacaccgatagcgaaggcagcttaccaggctatatctgacgctgaatcacgagagcgtgggtatcgaacaggattagataccctggtagtccacgccgtaaacgatgctcaccggctctatgcgataagacagtatggggctaatagaaataattaagtgagccacctggggagtacgtcggcaacgatgaaactcaaaggaattgacgggggcccgcacaagcggaggaacatgtggtttaattcgatgatacgcgaggaaccttacccgggtttaaatgtatgttgcattatgtagaaatacgtattttcttcggaactgcatacaaggtgctgcatggttgtcgtcagctcgtgccgtgaggtgtcgggttaagtcccataacgagcgcaacccttatgattagttgctaacggttcaagccgagcactctattcacactgccaccgtaaggtgcgaggaaggaggggatgatgtcaaatcagcacggcccttatatccggggctacacacgtgttacaatggtcggtacagcgggttgcatttacgtgagtaacagctaatcccaaaaatcggtctcagttcggattggagtctgcaactcgactccatgaagttggattcgctagtaatcgcacatcagccatggtgcggtgaatacgttcccgggccttgtacacaccgcccgtcaagccatgggagctgagggtgcctgaagttcgtaaccgcgaggagcggcctagggcaaacttggtaactggggctaagtcgtaacaaggtagctgtaccggaaggtgcggctggaacacctcctt

SEQ ID NO: 24 - DNA, 1476 bp, unidentified
  source 1..1476: mol_type = unassigned DNA; note = Pasteurellaceae sp.
attgaacgctggcggcaggcttaacacatgcaagtcgaacggtagcaggagaaagcttgctttcttgctgacgagtggcggacgggtgagtaatgcttgggaatctggcttatggagggggataactacgggaaactgtagctaataccgcgtaatatctttggattaaagaatgggacattagccattcgccataagatgagcccaagtgggattaggtagttggtggggtaagggcctaccaagccgacgatctctagctggtctgagaggatgaccagccacactggaactgagacacggtccagactcctacgggaggcagcagtggggaatattgcgcaatgggggcaaccctgacgcagccatgccgcgtgaatgaagaaggccttcgggttgtaaagttctttcggtggtgaggaaggcttaagtgctaatagtacttaagattgacgttagccacagaagaagcaccggctaactccgtgccagcagccgcggtaatacggagggtgcgagcgttaatcggaatgactgggcgtaaagggcacgcaggcggatagttaagtgagatgtgaaagccccgggcttaacctgggaattgcatttcagactggcaatctagagtattttagggaggggtagaattccacgtgtagcggtgaaatgcgtagagatgtggaggaataccgaaggcgaaggcagccccttgggaatatactgacgctcatgtgcgaaagcgtggggagcaaacaggattagataccctggtagtccacgctgtaaacggtgtcgatttggggattggactttaagtctggtgcccgaagctaacgtgataaatcgaccgcctggggagtacggccgcaaggttaaaactcaaatgaattgacgggggcccgcacaagcggtggagcatgtggtttaattcgatgcaacgcgaagaaccttacctactcttgacatccatggaatcttgtagagatacgagagtgccttcgggaaccatgagacaggtgctgcatggctgtcgtcagctcgtgttgtgaaatgttgggttaagtcccgcaacgagcgcaacccttatcctttgttgccagcgattaggtcgggaactcaaaggagactgccagtgataaactggaggaaggtggggatgacgtcaagtcatcatggcccttacgagtagggctacacacgtgctacaatggtgcatacagagggaagcaatatggcgacatggagcgaatctcacaaagtgcatctaagtccggattggagtctgcaactcgactccatgaagtcggaatcgctagtaatcgcgaatcagaatgtcgcggtgaatacgttcccgggccttgtacacaccgcccgtcacaccatgggagtgggttgtaccagaagtagatagcttaaccttcgggagggcgtttaccacggtatgattcatgactggggtgaagtcgtaacaaggta

SEQ ID NO: 25 - DNA, 1540 bp, Peptococcus sp.
  source 1..1540: mol_type = genomic DNA
gacaaatgctggcggcatgcctaacacatgcaagtcgaacggacaagttagtggaaatttttcggaatggaaactaatgcgttagtggcggatgggtgagtaacgcgtggataacctgcctctaaatggggaataactacgagaaatcgtagctaataccgcataatatctctttattgcatgatagagagaggaaagacggccttagtgctgtcgtttagagatggatccgcgtctgattagctagttggtgaggtaagggctcaccaaggcgacgatcagtagccggcctgagagggtgaacggccacattgggactgagacacggcccaaactcctacgggaggcagcagtggggaatcttccgcaatgggcgcaagcctgacggagcaatgccgcgtgagtgaagaaggccttcgggttgtaaaactctgtccttatcgaagagagagaagagtagtaactgacttttctaggacggtagatgaggaggaagccccggctaactatgtgccagcagccgcggtaaaacatagggggcaagcgttgtccggaatcactgggcgtaaagggcgcgcaggcggtttattaagtcaggtgtgaaagttcggggctcaaccccgtgattgcacctgatactgatagactagagtgttggagaggtaagtggaattcctagtgtagcggtggaatgcgtagatattaggaggaacaccagtggcgaaggcgacttactggccaataactgacgctgaggcgcgaaagcgtggggagcaaacaggattagataccctggtagtccacgccgtaaacgatgagtactaggtgttggttgtttcgatacaatcggtgccgtagttaacacaataagtactccgcctggggagtacggtcgcaagactgaaactcaaaggaattgacgggggcccgcacaagcggtggagcatgtggtttaattcgacgcaacgcgaagaaccttaccaagacttgacatcctcttgaccggatatgtaatgtatccttttcttcggaacaagagagacaggtggtgcatggttgtcgtcagctcgtgtcgtgagatgttgggttaagtcccgcaacgagcgcaacccctatagttagttgccatcattaagttgggcactctagcaagactgccggtgacaaaccggaggaaggtggggatgacgtcaaatcatcatgccccttgtgtcttgggctacacacgtgctacaatggttggtacagagggcagcgaaggagtgatctggagcgaatcccaaaaagccaatctcagttcggattgtaggctgcaactcgcctacatgaagtcggaatcgctagtaatcgcaggtcagcatactgcggtgaatacgtacccgggccttgtacacaccgcccgtcacaccacgaaagttggtaacacccgaagtcggtggtctgacccgcaagggaggaagccgcccacggtgggatcgatgattggggtgaagtcgtaacaaggtagccgtatcggaaggtgcggctggatcacctcctt

SEQ ID NO: 26 - DNA, 1611 bp, unidentified
  source 1..1611: mol_type = unassigned DNA; note = Peptostreptococcaceae sp.
gatgaacgctggcggcgtgcttaacacatgcaagtcgaacggacctggtttttagcacaaattgcttatgcctatcagcatgggcggatgcggaccctttgtccatacgaaagagcatccgtctacgcgtaaaaagcgtgataaaccatgcaagcagaacggaaagtaatgagtgatttgtgctgaaaatcaggttagtggcggacgggtgcgtaacacgtgggcaacctgccctgcttttcggaataacgccgggaaaccgtcgctaatgccgaatgatatgagaagaaggcatcttcttttcatcaaagaatttcgaagcaggatgggcccgcgccggattagctggttggtgaggtaacggctcaccaaggcgacgatccgtagccgacctgagagggtgatcggccacactggaactgagacacggtccagactcctacgggaggcagcagtggggaatattgcacaatgggcgaaagcctgatgcagcaacgccgcgtgagcgatgaaggctttcgagtcgtaaagctctgtcgcaggggaagaaaaagacggtaccctgtgagaaagccccggctaactacgtgccagcagccgcggtaatacgtagggggcaagcgttatccggaattactgggcgtaaagggtgcgtaggcggcttttcaagtcaaggcttaaaggctaacggctcaaccgtagtaaggccttgaaactggagagcttgagtatcggagaggaaagtggaattcccagtgtagcggtaaaatgcgtagatattgggaggaacaccagtggcgaaggcgactttctggacgatgactgacgctgaggcacgaaagcgtggggagcaaacaggattagataccctggtagtccacgccgtaaacgatgagtactaggtgtcaggtgtcaaagcttggtgccgcagttaacacagtaagtactccgcctggggagtacgctcgcaagagtgaaactcaaaggaattgacggggacccgcacaagtagcggagcatgtggtttaattcgaagcaacgcgaagaaccttacctaagcttggcatcccgttgaccggggcttaatcgcccctttcccttcggggacagcggagacaggtggtgcatggttgtcgtcagctcgtgtcgtgagatgttgggttaagtcccgcaacgagcgcaacccttgtttttagttgccagcggtttggccgggcactctaaaaagactgccgaggataactcggaggaaggtggggatgacgtcaaatcatcatgccccttatgcttagggctacacacgtgctacaatggctgttacaaagggccgcgaggccgcaaggcggagccaatcccataaaaacagtctcagttcggattgcaggctgaaactcgcctgcatgaagtcggagttactagtaatcgcgaatcagcatgtcgcggtgaatgcgttcccgggtcttgtacacaccgcccgtcacaccatggaagcttggggcacccaaagtccgtgatccaacgaaagaggaagcggcctaaggtgaaacaagtgactggggtgaagtcgtaacaaggtagccgtatcggaaggtgcggctggatcacctcctt

SEQ ID NO: 27 - DNA, 1501 bp, unidentified
  source 1..1501: mol_type = unassigned DNA; note = Peptostreptococcaceae sp.
gatgaacgctggcggcgtgcttaacacatgcaggtcgaacgaagcaggaaggaagaagtttttcgaagccgattttttccttgcttagtggcggacgggtgcgtaacacgtaggtaacctgccctgttcattggaataacttcgggaaaccggtgctaatgccaaataacatcgcctgttggcatcaagaggcgatcaaagaatatcggaacaggatgggcctgcgccggattagctagttggtgaggtaatagctcaccaaggcaacgatccgtagccgacctgagagggcgatcggccacactggaactgagacacggtccagactcctacgggaggcagcagtggggaatattgcacaatgggcgaaagcctgatgcagcaacgccgcgtgagcgaagaaggctttcgagtcgtaaagctctgtcctatgagaagatgatgacggtaccataggaggaagccccggctaactacgtgccagcagccgcggtaatacgtagggggcaagcgttatccggaattactgggcgtaaagggtgcgtaggcggctgttcaagtcaacgctgaaaggctaacggctcaaccgtagtaaggcgttgaaactggagagcttgagtatcggagaggaaagtagaattcccagtgtagcggtgaaatgcgtagatattgggaggaataccggtggcgaaggcgactttctggacgaaaactgacgctgaggcacgaaagcgtggggagcaaacaggattagataccctggtagtccacgccgtaagcgatgagtactaggtgtcaggtgtcaaaacctggtgccgcagttaacacagtaagtactccgcctggggagtacgctcgcaagagtgaaactcaaaggaattgacggggacccgcacaagtagcggagcatgtggtttaattcgaagcaacgcgaagaaccttacctaagcttgacatcccgttgaccggggtttaatcgcccctttcccctcggggacagcggagacaggtggtgcatggttgtcgtcagctcgtgtcgtgagatgttgggttaagtcctgcaacgagcgcaacccctatttttagttgccagcggtatggccgggcactctaaaaagactgccggggataacccggaggaaggtggggatgacgtcaaatcatcatgccccttatgcctagggctacacacgtgctacaatggctgttacaaagggctgcgacgccgcaaggcgaagccaatcccataaaaacagtctcagttcggattgcaggctgaaactcgcctgcatgaagtcggagttactagtaatcgcaaatcagcatgttgcggtgaatgcgttcccgggtcttgtacacaccgcccgtcacaccatggaagcttggggcacccaaagtccgtcgtctaacttcggaagaagcggcctaaggtgaaacaagtgactggggtgaagtcgtaacaaggtagccgtatcggaaggtgcggctggatcacctcctt

SEQ ID NO: 28 - DNA, 1500 bp, unidentified
  source 1..1500: mol_type = unassigned DNA; note = Peptostreptococcaceae sp.
gatgaacgctggcggcgtgcctaacacatgcaagtcgagcgagaatcttggaagtggagacttcggtcgaagcgactaagaggaaagcggcggacgggtgagtaacgcgtgggcaacctgcctcttactagaggatagccaccggaaacggtgattaatactgtatgaagtgcatttatcgcatgatagatgtcccaaagatttatcggtaagagatgggcccgcgtctgattagctagttggcaaggtaacggcttaccaaggcgacgatcagtagccgacctgagagggtgaacggccacactggaactgagacacggtccagactcctacgggaggcagcagtggggaatattcggcaatggggggaaccctgaccgagcaacgccgcgtgagtgattgaaggtcttcggactgtaaaactctgttgaatgggaagataatgacggtaccatttgaggaagccccggctaactacgtgccagcagccgcggtaatacgtagggggcgagcgttatccggaatcactgggcgtaaagggtgcgtaggcgggacatcaagccaggggtgaaaggcagtggctcaaccattgtaagcctttggaactgatgttcttgagtgcaggagaggtaagtggaattcctagtgtagaggtaaaattcgtagatattaggaggaacaccggtggcgaaggcgacttactggactgtaactgacgctgatgcacgaaggcgtgggtagcaaacaggattagataccctggtagtccacgcggtaaacgatgaatactaggtattgggaatcaaatctcggtgccgcagctaacgcattaagtattccgcctggggagtacgcacgcaagtgtgaaactcaaaggaattgacggggacccgcacaagtagcggagcatgtggtttaattcgtggcaacgcgtagaaccttaccagatcttgacatcccaatgaatatactttaatcggtatagctcttcggagcattggagacaggtggtgcatggttgtcgtcagctcgtgtcgtgagatgttgggttaagtcccgcaacgagcgcaacccttgtgtttagttgttaccattaagttgaaccctctaaacagactgccgtggataacgcggaggaaggtggggatgaggtcaaatcatcatgccccttacgatctgggctacacacgtgctacaatggccggtacaacgggcagcgaaggagcgatccggagcaaaacctaaaaagccggtcccagttcggattgcaggctgcaactcgcctgcatgaagtcggagttactagtaatcgcggatcagaatgtcgcggtgaatgcgttctcgggtcttgtacacaccgcccgtcacaccatggaagttcggggcgcccgaagccggtggcctaaccttagggaaggagctgtcgaaggtgaaacgaatgactggggtgaagtcgtaacaaggtagccgtatcggaaggtgcggctggatcacctcctt

SEQ ID NO: 29 - DNA, 1506 bp, unidentified
  source 1..1506: mol_type = unassigned DNA; note = Peptostreptococcaceae sp.
gatgaacgctggcggcgtgcctaatacatgcaagtcgagcgagaacttattaaatggagacttcggtcaaagtgagataagggaaagcggcggacgggtgagtaatgcgtgggaaacctgccctttagagtgggatagcagttggaaacgactattaataccccatactcttgagttttcgcatgaagattcaaggaaagattttatcgctaaaggatggtcccacgtctgattagctagttggtgaggtaacggctcaccaaggcgacgatcagtagccgacctgagagggtgatcggccacactggaactgagacacggtccagactcctacgggaggcagcagtagggaatattgggcaatgggggcaaccctgacccagcaacgccgcgtgaatgatgaaggtcttcggatcgtaaaattcttttcttggggaagaacaaggtgacggtacccaaggaataagcaccggctaaccacgtgccagcagccgcggtaatacgtagggtgcaagcgttatccggaatcactgggcgtaaagggtgcgtaggcggcctatcaagtcagaagttaaagacaatggcttaaccattgaaagcttttgaaactgattggcttgaatacaggagaggcaagtggaattcctagtgtagcggtgaaatgcgtagatattaggaggaacaccggtggcgaaggcgacttgctggactgagattgacgctgaggcacgaaagcggggggagcgaacaggattagataccctggtagtccacgccgtaaacgatgaatactaggtgtcggctagaaatagtcggtgccggcgcaaacgcaataagtattccgcctggggagtacgcacgcaagtgtaaaactcaaaggaattgacggggacccgcacaagcagcggagcatgtagtttaattcgacgcaacgcgaaaaccttaccaagtcttgacatctggatgaccgcactttaacaggtgcctctcttcggagcatccaagacaggtggtgcatggttgtcgtcagctcgtgtcgtgagatgttgggttaagtcccgcaacgagcgcaacccttatttttagttgccatcaggtgaagctgggcactctaaagagactgccgtggacaacacggaggaaggtggggatgacgtcaaatcatcatgccccttatgatttgggctacaaacgtgctacaatggccggtacaacgggcagcgaagaggtgactcggagcgaatcctaaaaagccggtcccagttcggattgtaggctgcaactcgcctacatgaagttggagttgctagtaatcgtgaatcagcatgtcgcggtgaatgcgttcccgggtcttgtacacaccgcccgtcaaaccatggaagctgggggtacccgaagtcagtgatctaacccgcaagggaggaagctgccgaaggtaaaaccaatgactggggttaagtcgtaacaaggtagccgtatcggaaggtgcggctggatcacctcctt

SEQ ID NO: 30 - DNA, 1484 bp, unidentified
  source 1..1484: mol_type = unassigned DNA; note = Peptostreptococcaceae sp.
gatgaacgctggcggcgtgcctaacacatgcaagtcgagcgagaactttgagaaagaagcttcggtagatttttcaaagggaaagcggcggacgggtgagtaacgcgtaggcaacctgccccatacaaagggatagcaacgggaaactattattaaaaccttatgaagtaaagatgttgcatgacatttttaccaaagatttatcggtatgggatgggcctgcgtctgattagttagttggtgaggtaacggctcaccaaggcgacgatcagtagccgacctgagagggtgatcggccacattggaactgagacacggtccaaactcctacgggaggcagcagtggggaatattgcacaatgggcgaaagcctgatgcagcaacgccgcgtgaacgaagaaggcccttgggtcgtaaagttctgtccaaagggaagataatgacggtacctttggaggaagccccggctaactacgtgccagcagccgcggtaatacgtagggggcaagcgttatccggaattattgggcgtaaagggtacgtaggtggtcttttaagcgcagggtttaaggcaatggcttaaccattgtatgccttgcgaactggaggacttgagtgcaggaggggaaagcggaattcctagtgtagcggtgaaatgcgtagatattaggaggaacaccagcggcgaaggcggctttctggactgtaactgacactgaggtacgaaagcgtggggagcaaacaggattagataccctggtagtccacgccgtaaacgatgagcactagatgtcgggtagaaatattcggtatcgcagttaacgcaataagtgctccgcctggggagtacgcacgcaagtgtgaaactcaaaggaattgacggggacccgcacaagcagcggagcatgtggtttaattcgaagcaacgcgaagaaccttaccaggacttgacatccctctgacaggagtgtaatgactcttttctacggacagaggagacaggtggtgcatggttgtcgtcagctcgtgtcgtgagatgttgggttaagtcccgcaacgagcgcaacccttgtctttagttgccatcattaagttgggcactctaaagagactgccggggacaactcggaggaaggtggggatgacgtcaaatcatcatgccccttatgttctgggctacacacgtgctacaatggccggaacaaagagaagcgaaatcgtgaggtggagcgaatctcaaaaaccggccccagttcggattgcaggctgcaactcgcctgcatgaagtcggagttgctagtaatcgcagatcagaatgctgcggtgaatgcgttcccgggtcttgtacacaccgcccgtcacaccatggaagttgggggcgcccaaagttggtcggcagatagactacctaaggtgaaaccaatgactggggtgaagtcgtaacaaggtagccgtatcggaaggtgcggctggatcacctcctt

SEQ ID NO: 31 - DNA, 1500 bp, unidentified
  source 1..1500: mol_type = unassigned DNA; note = Peptostreptococcaceae sp.
gattaacgctggcggcgtgcgtaacacatgcaagtcgaacggtattttgaaaggaagagacttcggtcttggaaagtcaagagagagtggcgaacgggtgagtaacgcgtgagcaacctgtcttccacaaagggatagctgccggaaacggtaattaataccatatgaagttaacgaatcgcatgataaggtaactaaagagaaatcggtggaagatgggctcgcgtccgattagctggttggagagataacagctcaccaaggcaatgatcggtagccggcttgagagagtgaacggccacactggaactgagagacggtccagactcctacgggaggcagcagtggggaattttgcacaatggggggaaccctgatgcagcgacgccgcgtgagcgaagaaggaattcgtttcgtaaagctctgtcctatggaaagataatgacggtaccataggaggaagccccggctaactacgtgccagcagccgcggtaatacgtagggggcgagcgttgtccggaattattgggcgtaaagggtacgcaggcggtctaagaagtcagatgttaaagcgtagggcttaaccctataaagcatatgaaactgatagacttgagtgaaggagaggaaagtggaattcctggtgtagcggtgaaatgcgtagatatcaggaggaataccggtggcgaaggcgactttctggactttaactgacgctgaggtacgaaagcgcggggagcaaacaggattagataccctggtagtccgcgccgtaaacgatgaatgctaggtattgggagtcaaatttcagtgccgaagtaaacacattatgcattccgcctggggagtacggtggcaacactgaaactcaaaggaattgacggggacccgcacaagcagcggagcatgtggtttaattcgatgcaacgcgaagaaccttaccggggcttgacatatacttgaagtatgtagagatacatatgattcttcggaaacaggtaaacaggtggtgcatggttgtcgtcagctcgtgtcgtgagatgttgggttaagtcccgcaacgagcgcaacccctatgcttagttaccagcagtaagatggggactctaagcagactgccgatgataaatcggaggaaggcggggaagacgtcaaatcatcatgccctttatgtcccgggctacacacgtgctacaatggtcggtacagagggatgcgacatagagatatgaagcgaaacccaaaaagccggtcccagttcggattgtaggctgcaattcgcctacatgaagtcggagttgctagtaatcgcaaatcagaatgttgcggtgaatgcgttcccgggtcttgtacacaccgcccgtcacaccatgggagttaaaaatgcccgaagccggtgagctaaccgcaagggagcagccgtcgaaggtagggttaatgactggggtgaagtcgtaacaaggtagccgtatcggaaggtgcggctggatcacctcctt

SEQ ID NO: 32 - DNA, 1499 bp, unidentified
  source 1..1499: mol_type = unassigned DNA; note = Peptostreptococcaceae sp.
gatgaacgttggcgacgtgcctaacacatgcaagtcgagcggtgaaaagcggacttaatcttcggaaagaggaaacttggaacagcggcggacgggtgagtaatacatgagcaacctgccttggacagagggatagccgtgggaaaccgcgattaatacctcataagaccccactatcgcatgatagagaggttaaagattcatcggtccaagatgggctcatgacccattagctagttggtgagataacagcccaccaaggcgacgatgggtagccggcttgagagagtgaccggccacattggaactgagacacggtccagactcctacgggaggcagcagtggggaatattgcacaatggaggaaactctgatgcagcgacgtcgcgtgagcgaagaaggttttcgaatcgtaaagctctgtcctatgggaagaagaaagacggtaccatagaagaaagccccggctaactacgtgccagcagccgcggtaatacgtagggggcgaacgttatccggaatgattgggcgtaaagggtgcgtaggcggacctgtaagtcaggtgtgaaaggcatgggctcaacccatgtaagcacttgaaactgcgggccttgagggaaggagaggtaagtggaattcctagtgtagcggtgaaatgcgtagatattaggaggaataccggtggcgaaggcgacttactggacttttactgacgctgaggtacgaaagcgtggggagcaaacaggattagataccctggtagtccacgccgtaaatggtgagtgctaggtgtcgggagtcaatctcggtgccgacgttaacacattaagcactccgcctggggagtacgcacgcaagtgtgaaactcaaaggaattgacggggacccgcacaagcagcggagcatgtggtttaattcgatgcaacgcgaaaaaccttaccaggacttgacatatggatgcccggtatagagatataccctttcttcggaacattcatacaggtggtgcatggttgtcgtcagctcgtgtcgtgagatgttgggttaagtcccgcaacgagcgcaacccctattcttagttactaacaggtcaagctgaggactctaaggagactgccggtgacaaaccggaggaagatggggatgacgtcaaatcatcatgccctttatgtcctgggctacacacgtgctacaatggtcggtacagaaggaagcgagatagcgatatcgagcgaacccccaaagccgatctcagttcggattgttctctgcaactcgagaacatgaagccggagttgctagtaattgcgaatcagaatgtcgcagtgaatgcgttcccgggtcttgtacacaccgcccgtcacaccatgggagttggtaatacccgaagcctgtgagccaacctgcaaaggaagcagcagtcgaaggtaggatcaatgactggggtgaagtcgtaacaaggtagccgtatcggaaggtgcggctggatcacctcctt

SEQ ID NO: 33 - DNA, 1517 bp, Peptostreptococcus sp.
  source 1..1517: mol_type = genomic DNA
gatgaacgctggcggcgtgcctaacacatgcaagtcgagcgcggttgtgcttagtattgagtgttttattgatataaaacattgaattctatgcacaactgagcggcggacgggtgagtaacgcgtgggtaacctgccctatacacatggataacatactgaaaagtttactaatacatgataaaatagtttttcggcatcgaagaattatcaaagtgtttgcggtataggatggacccgcgtctgattagctagttggtgagataactgcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgaacgatgaaggtcttcggatcgtaaagttctgttgcaggggaagataatgacggtaccctgtgaggaagccccggctaactacgtgccagcagccgcggtaatacgtagggggctagcgttatccggatttactgggcgtaaagggtgcgtaggtggtctttcaagtcggtggttaaaggctacggctcaaccgtagtaagcctccgaaacggttagacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacaggattagataccctggtagtccacgccgtaaacgatgagtactaggtgtcgggggttacccccctcggtgccgcagctaacgcattaagtactccgcctggggagtacgcacgcaagtgtgaaactcaaaggaattgacggggacccgcacaggtagcggagcatgtggtttaattcgaagcaacgcgaagaaccttacctaagcttgacatccctcggaccggtgtttaatcacacctttccttcgggactgaggagacaggtggtgcatggttgtcgtcagctcgtgtcgtgagatgttgggttaagtcccgcaacgagcgcaacccttgtctttagttgccatcattaagttgggcactctagagagactgccagggacaacctggaggaaggtggggatgacgtcaaatcatcatgccccttatgcttagggctacacacgtgctacaatgggtggtacagagggttgccaaaccgtgaggtggagccaatcccttaaagccactctcagttcggattgtaggctgaaactcgcctacatgaagctggagttactagtaatcgcagatcagaatgctgcggtgaatgcgttcccgggtcttgtacacaccgcccgtcacaccatgggagtcggaagcacccgaagccgattatctaaccgcaaggaggagatcgtcgaaggtggcgtcgataactggggtgaagtcgtaacaaggtagccgtatcggaaggtgcggctggatcacctcctt

SEQ ID NO: 34 - DNA, 1490 bp, Peptostreptococcus sp.
  source 1..1490: mol_type = genomic DNA
gatgaacgctggcggcgtgcctaacacatgcaagtcgagcgcgactgatttgatgcttgcatcgatgaaagttgagcggcggacgggtgagtaacgcgtgggcaacctgccctgtacacatggataacatactgaaaagtttactaatacatgataatatagtttttcggcatcgaagaattatcaaagtgttagcggtacaggatgggcccgcgtctgattagctagttggtgagataactgcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgaacgatgaaggtcttcggatcgtaaagttctgttgcaggggaagacaatgacggtaccctgtgaggaagccccggctaactacgtgccagcagccgcggtaatacgtagggggctagcgttatccggatttactgggcgtaaagggtgcgtaggtggtccttcaagtcggtggttaaaggctacggctcaaccgtagtaagcctccgaaactgttggacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacaggattagataccctggtagtccacgctgtaaacgatgagtactaggtgtcgggggttacccccctcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaagcttgacatccctcggaccggtgtttaatcacacctttccttcgggactgaggtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcttagggctacacacgtgctacaatgggtggtacagagggttgccaaaccgtgaggtggagctaatcccttaaagccattctcagttcggattgtaggctgaaactcgcctacatgaagctggagttactagtaatcgcagatcggaatgctgcggtgaatgcgttcccgggtcttgtacacaccgcccgtcacaccatgggagtcggaaacacccgaagccgattatctaaccgcaaggaggaagtcgtcgaaggtggcgtcgataactggggtgaagtcgtaacaaggtagccgtatcggaaggtgcggctggatcacctcctt

SEQ ID NO: 35 - DNA, 1469 bp, Porphyromonas cangingivalis
  source 1..1469: mol_type = genomic DNA
gatgaacgctagcgataggcttaacacatgcaagtcgaggggcagcataattttagcttgctaagattgatggcgaccggcgcacgggtgcgtaacgcgtatgcaacctgcctgtcagtggggaataacccggagaaatccggactaataccgcatacactttggtttccgcatggggactttaggaaagatttattgctgacagataggcatgcgttccattagatagttggtgaggtaacggctcaccaagtcgacgatggataggggagctgagaggcttaccccccacactggaactgagacacggtccagactcctacgggaggcagcagtgaggaatattggtcaatgggcgagagcctgaaccagccaagtcgcgtgaaggaagacggtcctatggattgtaaacttctttagtaggtgagtacaagcagttacgtgtagctgtaagcaagtaacctaagaataagtatcggctaactccgtgccagcagccgcggtaatacggaggatacgagcgttatccggatttattgggtttaaagggtgcgtaggtggctttataagtcagtggtgaaaagctgtggctcaaccatagtcttgccgttgaaactgtggagcttgagtgcggatggggtaggcggaacgcgtagtgtagcggtgaaatgcatagatattacgcagaactccgatcgcgaaggcagcttaccaaaccgcaactgacactgaagcacgaaagcgtgggtatcaaacaggattagataccctggtagtccacgcagtaaacgatgattactcggagtatgcgatataatgtatgctcccaagggaaaccgataagtaatccacctggggagtacgccggcaacggtgaaactcaaaggaattgacgggggcccgcacaagcggaggaacatgtggtttaattcgatgatacgcgaggaaccttacccgggattgaaatgtacatgacggttgggcgagagcctggcttcccttcggggcatgtatgtaggtgctgcatggttgtcgtcagctcgtgccgtgaggtgtcggcttaagtgccataacgagcgcaacccacatcgtcagttactaacaggtagagctgaggactctgacgagactgccgtcgtaaggcgcgaggaaggtgtggatgacgtcaaatcagcacggcccttacatccggggcgacacacgtgttacaatggtagggacaaagggcagctacctggcgacaggatgcgaatctccaaaccctatctcagttcggatcggagtctgcaactcgactccgtgaagctggattcgctagtaatcgcgcatcagccatggcgcggtgaatacgttcccgggccttgtacacaccgcccgtcaagccatggaagtcgggggtacctgaagtgcgtcaccgcaaggatcgcacgagggtaagaccggtaactggggctaagtcgtaacaaggt

SEQ ID NO: 36 - DNA, 1505 bp, Porphyromonas gingivicanis
  source 1..1505: mol_type = genomic DNA
gatgaacgctagcgataggcttaacacatgcaagtcgaggggcagcataattttagcttgctaagattgatggcgaccggcgcacgggtgcgtaacgcgtatgcaacctgcctgtaattaggggatagctcggtgaaagtcgaattaataccctatattctatatttttcgcatgattgatataggaaagatttattgattacagatgggcatgcgttccattagcttgttggtaaggtaacggcttaccaaggcaacgatggatagggggactgagaggttgaccccccacactggtactgagacacggaccagactcctacgggaggcagcagtgaggaatattggtcaatgggcgcaagcctgaaccggccaagtcgcgtgaaggacgacggtcctatggattgtaaacttcttttgtagaggaataatgatggccacgagtggctgaagggaatgtactctacgaataagtatcggctaactccgtgccagcagccgcggtaatacggaggatacgagcgttatccggatttattgggtttaaagggtgcgtaggtggcgaattaagtcagtggtgaaaagctgcagctcaactgtagtcttgccgttgaaactggtttgcttgagaggagacgaggtatgcggaatgtgtggtgtagcggtgaaatgcatagatatcacacagaacgccgattgcgaaggcagcgtaccaggctctttctgacactgaagcacgaaagcgtgggtatcaaacaggattagataccctggtagtccacgctgtaaacgatgaatactagatttttgcgatatacagtaagagtctaagcgaaagcgataagtattccacctggggagtacgccggcaacggtgaaactcaaaggaattgacgggggcccgcacaagcggaggaacatgtggtttaattcgatgatacgcgaggaaccttacctgggattgaaatttaggagaacgatttatgaaagtagattttcccttcggggctcctgagcaggtgctgcatggttgtcgtcagctcgtgccgtgaggtgtcggcttaagtgccataacgagcgcaacccgcgtcgatagttactaacaggtaatgctgaggactctatcgagacagccgtcgtaagacgagaggaaggggcggatgacgtcaaatcagcacggcccttacatccagggcgacacacgtgttacaatggcaaggacaaagggaagccacatagcgatatggagcagatcctcaaaccttgtcccagttcggatcggagtctgcaactcgactccgtgaagctggattcgctagtaatcgcgcatcagccatggcgcggtgaatacgttcccgggccttgtacacaccgcccgtcaagccatgggagtcgggggtacctgaagagtgtaaccgtaaggagcgcttaagggtaataccggtgactggggctaagtcgtaacaaggtagccgtaccggaaggtgtggctggatcacctcctt

SEQ ID NO: 37 - DNA, 1504 bp, Porphyromonas gulae
  source 1..1504: mol_type = genomic DNA
gatgaacgctagcgataggcttaacacatgcaagtcgaggggcagcatgaacttagcttgctaagtttgatggcgaccggcgcacgggtgcgtaacgcgtatgcaacttgccttacagagggggataacccgttgaaagacggactaataccgcatacacttgcttggttgcatgatcgggcaaggaaatatttatagctgtaagataggcatgcgtcccattagctggttggtgaggtaacggctcaccaaggcaacgatgggtaggggaactgagaggtttatcccccacactggtactgagacacggaccagactcctacgggaggcagcagtgaggaatattggtcaatgggcgagagcctgaaccagccaagtcgcgtgaaggatgactgtcctaaggattgtaaacttcttttatacgggaataacgggcgatacgtgtattgctgtgaatgtaccgtaagaataagcatcggctaactccgtgccagcagccgcggtaatacggaggatgcgagcgttatccggatttattgggtttaaagggtgcgtaggttgttcggtaagtcagcggtgaaaactgagcgctcaacgttcagcgtgccgttgaaactgccgggcttgagttcagtggcggcaggcggaattcgtggtgtagcggtgaaatgcatagatatcacgaggaactccgattgcgaaggcagcttgccatactgcgactgacactgaagcacgaaggcgtgggtatcaaacaggattagataccctggtagtccacgcagtaaacgatgattactaggagtttgcgatataccgtcaagcttccacagcgaaagcgttaagtaatccacctggggagtacgccggcaacggtgaaactcaaaggaattgacgggggcccgcacaagcggaggaacatgtggtttaattcgatgatacgcgaggaaccttacccgggattgaaatgtagacgacggatggtgaaagccgtcttcccttcggggcgtctatgtaggtgctgcatggttgtcgtcagctcgtgccgtgaggtgtcggcttaagtgccataacgagcgcaacccacatcggtagttgctaacaggtttagctgaggactctaccgagactgccgtcgtaaggcgtgaggaaggtgtggatgacgtcaaatcagcacggcccttacatccggggctacacacgtgttacaatgggagggacaaagggcagctaccgggcgaccggatgcgaatctcgaaacccttccccagttcggatcggagtctgcaactcgactccgtgaagctggattcgctagtaatcgcgcatcagccatggcgcggtgaatacgttcccgggccttgtacacaccgcccgtcaagccatgggagtcgggggtacctgaagggcgtaaccgcaaggggcgcactagggtaataccggtgactggggctaagtcgtaacaaggtagccgtaccggaaggtgcggctggaacacctcctt

SEQ ID NO: 38 - DNA, 1469 bp, Porphyromonas macacae
  source 1..1469: mol_type = genomic DNA
gatgaacgctagcgataggcttaacacatgcaagtcgaggggcagcatggtcttagcttgctaagactgatggcgaccggcgcacgggtgcgtaacgcgtatgtaacttgcctgatagaaagggataacccggtgaaagtcggactaataccttatggtcttgggttattgcatgatgattcaagtaaagattaattgctatcagataggcatgcgttccattagttagttggtgaggtaacggctcaccaaggcgacgatggataggggaactgagaggtttatcccccacactggtactgagacacggaccagactcctacgggaggcagcagtgaggaatattggtcaatgggcgagagcctgaaccagccacgtcgcgtgaaggaagacggttctatggattgtaaacttcttttatagtggattaaagttatccacgcgtggatatttgcaagtaccctatgaataagcatcggctaactccgtgccagcagccgcggtaatacggaggatgcgagcgttatccggatttattgggtttaaagggtgcgtaggtggcctgataagtcgggggtgaaaagctgtggctcaaccacagtcttgccttcgaaactgtttggcttgagtatagatgaagtaggcggaatttgtggtgtagcggtgaaatgcatagatatcacgaggaactccgattgcgaaggcagcttactaagttatgactgacactgaagcacgaaagcgtgggtatcaaacaggattagataccctggtagtccacgcagtaaacgatgataactgggcgtatgcgatatacagtatgctcctgagcgaaagcgttaagttatccacctggggagtacgccggcaacggtgaaactcaaaggaattgacgggggcccgcacaagcggaggaacatgtggtttaattcgatgatacgcgaggaaccttacccgggattgaaatttagcggactatgtatgaaagtacatatcctgtcacaaggccgctaagtaggtgctgcatggttgtcgtcagctcgtgccgtgaggtgtcggcttaagtgccataacgagcgcaacccacgttgtcagttactatcgggtaaagccgaggactctgacaagactgccgtcgtaaggcgcgaggaaggtgtggatgacgtcaaatcagcacggcccttacatccggggcgacacacgtgttacaatggtggggacaaagggcagctacttggtgacaagatgcgaatctccaaaccccatcccagttcggatcgtagtctgcaactcgactatgtgaagctggattcgctagtaatcgcgcatcagccatggcgcggtgaatacgttcccgggccttgtacacaccgcccgtcaagccatgggagtcgggagtacctaaagcacgtaaccgcgaggggcgtgttagggtaataccggtgactggggctaagtcgtaacaaggt

SEQ ID NO: 39 - DNA, 1469 bp, Porphyromonas sp.
  source 1..1469: mol_type = genomic DNA
gatgaacgctagcgataggcttaacacatgcaagtcgaggggcagcatagagtcagcttgctgatttagatggcgaccggcggatgggtgcgtaacgcgtatgcaacttacctgtcagagggggataacccggagaaattcgaactaataccgcatatacttgagatgctgcatggtatttcaaggaaatatttatagctgacagataggcatgcgatccattagctagtaggtgaggtaacggctcacctaggcgacgatggataggggaactgagaggttaaacccccacactggtactgagacacggaccagactcctacgggaggcagcagtgaggaatattggtcaatgggcgagagcctgaaccagccaattcgcgtgaaggacgactgccctatgggttgtaaacttctttagtaggggattaaagtttgccttgcgaggcaatttgcaagtaccctaagaataagtatcggctaactccgtgccagcagccgcggtaatacggaggatacgagcgttatccggatttattgggtttaaagggtgcgtaggtggctttttaagtcagtggtgaaaagctgtggctcaaccatagtcttgccgttgaaactgaggagcttgagtgtagatgctgtaggcggaacgcgtagtgtagcggtgaaatgcatagatattacgcagaactccgattgcgaaggcagcttacaaagttacaactgacactgaagcacgagagcgtgggtatcaaacaggattagataccctggtagtccacgccgtaaacgatgattactaagagtatgcgatataatgtatgttctacagcgaaagcgttaagtaatccacctggggagtacgtcggcaacgatgaaactcaaaggaattgacgggggcccgcacaagcggaggaacatgtggtttaattcgatgatacgcgaggaaccttacccgggattgaaatgtgcatgacgtaaattggagacagtttatacccttcggggcatgtatgtaggtgctgcatggttgtcgtcagctcgtgccgtgaggtgtcggcttaagtgccataacgagcgcaacccacattgtcagttactaacaggtagagctgaggactctggcgagactgccggcgtaagccgtgaggaaggtgtggatgacgtcaaatcagcacggcccttacatccggggcgacacacgtgttacaatggtgaggacaaagggcagctacctggcgacagggtgcgaatctccaaacctcatctcagttcggatcggagtctgcaactcgactctgtgaagctggattcgctagtaatcgcgcatcagccatggcgcggtgaatacgttcccgggccttgtacacaccgcccgtcaagccatggaagtcgggagtacctgaagtacgtcaccgcgaggatcgtactagggtaataccggtaactggggctaagtcgtaacaaggt

SEQ ID NO: 40 - DNA, 1461 bp, unidentified
  source 1..1461: mol_type = unassigned DNA; note = Synergistales sp.
gatgaacgctggcggcgtgcctaacacatgcaagtcgaacggggttatattggttagtttactaagcgatatgacttagtggcggacgggtgagtaacgcgtgaggacctatcccaagctgggggacaacagctggaaacggttgctaataccgcataagcgcataatctgcgtaaaaggagagatccggcatgggaggggctcgcgtcctatcagctagtaggtggggtaaaggcctacctaggcgatgacgggtagccggcctgagagggcgcacggccacactgggactgagatacggcccagactcctacgggaggcagcagtggggaatattgggcaatggggggaaccctgacccagcgacgccgcgtgagtgaagaagtccttcgggacgtaaagctctgttgtgtgggaagaagaggatgacggtaccacacgaggaagccccggcaaactacgtgccagcagccgcggtaatacgtagggggcgagcgttgtccggaattactgggcgtaaagggcacgcaggcggaatggtaagtcagttgtgaaaggctgtggctcaaccacagggagacgactgatactgtcagtctagagtatgtgagagggaagtggaattcccggtgtagcggtgaaatgcgtagatatcgggaggaacaccagtggcgaaggcggcttcatggcacataactgacgctcatgtgcgaaagctagggtagcgaacggaattagatactccggtagtcctagccgtaaacgatggatactaggtgtgggtgtcgcagggcatccgtgccggagtcaacgcgttaagtatcccgcctggggactacggccgcaaggctgaaactcaaaggaattgacgggggcccgcacaagcggtggagcacgtggtttaattcgatgcaaaccgaagaaccttacctgggtttgacatgtatatgttagagaagcgagagcggatcgaccacagtttactgtggagtatacacaggtgctgcatggctgtcgtcagctcgtgtcgtgagatgttgggttaagtcccgcaacgagcgcaacccctgttgtcagttgctaacggttagagccgagcactctggcgagactgccgtcgacaagacggaggaaggtggggacgacgtcaagtcatcatggcctttatgtccagggcgacacacgtgctacaatggtatgcacagagggaagcgaagtggtgacatggagcggatccaaggaagcatatctcagttcggattgtagtctgcaactcggctacatgaagccggaatcgctagtaatcgcagatcagccaagctgcggtgaatacgttcccgggccttgtacacaccgcccgtcacaccactcgagttgtctgcacccgaagccagtggcataaccgcaagggatgagctgtctaaggtgtggggggtaaggggggtgaagtcgtaacaaggt

SEQ ID NO: 41 - DNA, 1497 bp, Tannerella forsythia
  source 1..1497: mol_type = genomic DNA
gatgaacgctagcgataggcttaacacatgcaagtcgaggggcagcgagggtagcaatacctgtcggcgaccggcgcacgggtgagtaacgcgtatgtaacctgcccgcaacagggggataacccggcgaaagtcggactaataccgcataaaacaggggtcccgcatgggaatatttgttaaagatttatcggttgcggatgggcatgcgtaccattaggtagttggtgaggtaacggctcaccaagccttcgatggttaggggttctgagaggaaggtcccccacactggtactgagacacggaccagactcctacgggaggcagcagtgaggaatattggtcaatgggcgagagcctgaaccagccaagtcgcgtgaaggatgactgccctatgggttgtaaacttcttttacaagggaataaaatgagatacgtgtattttattgcatgtaccttgggaataagcatcggctaactccgtgccagcagccgcggtaatacggaggatgcgagcgttatccggatttattgggtttaaagggtgcgtaggtgggctgttaagtcagcggtgaaagtttgtcgcttaacgataaaattgccgttgaaactggtagtcttgagtatagatgaagtaggcggaatgcgtggtgtagcggtgaaatgcatagagatcacgcagaactccgattgcgaaggcagcttactaaggtataactgacactgaagcacgaaagcgtgggtatcaaacaggattagataccctggtagtccacgcagtaaacgatgattactaggagtttgcgatatacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggattgaaatgtagacgacggacagtgagagctgtcttcccttcggggcgtctatgtaggtgctgcatggttgtcgtcagctcgtgccgtgaggtgtcggcttaagtgccataacgagcgcaaccctgactgtcagttgctaacaggttaagctgaggactctggcgggactgccggcgtaagctgtgaggaaggttgggatgacgtcaaatcagcacggcccttacatccggggcgacacacgtgttacaatggcagggacaaagggcagctaccgggcgaccggatgccaatctccaaaccctgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcgaggatcggcctagggtaaaactggtgactggggctaagtcgtaacaaggtagccgtaccggaaggtgcggctggaacacctcctt